(19) United States
(12) Patent Application Publication
(10) Pub. No.: US 2024/0002361 A1
(43) Pub. Date: Jan. 4, 2024

(54) COMPOSITIONS AND METHODS FOR BLOCKING NEUROPILIN RECEPTOR 1 FOR THE TREATMENT OF PAIN AND PREVENTION OF VIRAL ENTRY

(71) Applicant: Arizona Board of Regents on Behalf of the University of Arizona, Tucson, AZ (US)

(72) Inventors: Rajesh Khanna, Tucson, AZ (US); Aubin Moutal, Tucson, AZ (US); Samantha Perez-Miller, Tucson, AZ (US)

(21) Appl. No.: 18/253,891

(22) PCT Filed: Nov. 17, 2021
(86) PCT No.: PCT/US2021/059667

§ 371 Date: May 22, 2023

Related U.S. Application Data
(60) Provisional application No. 63/117,336, filed on Nov. 23, 2020.

(51) Int. Cl. C07D 401/12; C07D 239/22; C07D 403/12; C07D 403/04; C07D 401/14; C07D 413/04; A61P 31/14

(52) U.S. Cl. CPC C07D 401/12; C07D 239/22; C07D 403/12; C07D 403/04; C07D 401/14; C07D 413/04; A61P 31/14

(57) ABSTRACT

Disclosed are compositions and methods for treating, preventing, or reducing neuropathic pain or severe acute respiratory syndrome coronavirus 2 (SARS-CoV-2) infection in a patient. The compositions contain, or the methods involve, compounds that inhibit neuropilin receptor 1 signaling. The compounds are present in amounts effective to treat, prevent, or reduce one or more symptoms associated with neuropathic pain or SARS-CoV-2 in a patient.

### CROSS-REFERENCE TO RELATED APPLICATION

This application claims benefit of and priority to U.S. Provisional Application No. 63/117,336 filed Nov. 23, 2020, the contents of which is incorporated herein by reference in its entirety.

### FIELD OF THE INVENTION

The disclosed methods and compositions are generally in the field of modulating receptor signaling, and specifically in the area of inhibiting neuropilin receptor 1 (NRP-1) signaling.

### BACKGROUND OF THE INVENTION

Neuropilins (NRPs) are cell surface receptors for secreted glycoproteins with roles in neural outgrowth, cardiovascular development, immune response, as well as tumor growth and vascularization (Pellet-Many, et al., Biochem J 2008, 411 (2), 211-26; Plein, et al., Microcirculation 2014, 21(4), 315-23). One isoform of NRPs is neuropilin receptor 1 (NRP-1). NRP-1 has a co-receptor, vascular endothelial growth factor receptor 2 (VEGFR2). Binding of a peptide ligand, vascular endothelial growth factor (VEGF) to VEGFR2 leads to receptor dimerization and activation of downstream signaling pathways.

Recently, VEGF-A has been shown to be pro-nociceptive (Llorian-Salvador and Gonzalez-Rodriguez, Frontiers in Pharmacology 2018, 9, 1267). It has also been shown that NRPs are entry points for several viruses into cells (Lambert, et al., Blood 2009, 113 (21), 5176-85; Wang, et al., Nat. Commun. 2015, 6, 6240; Cantuti-Castelvetri, et al., bioRriv 2020, 2020.06.07.137802; Daly, et al., bioRriv 2020, 2020.06.05.134114). Therefore, targeting NPRs for the treatment, prevention, or reduction of pain or viral entry remains an area of active research and an unmet need.

Therefore, it is an object of the invention to provide compounds and methods for treating, preventing, or reducing pain, such as neuropathic pain in a patient.

It is also an object of the invention to provide compounds and methods for treating, preventing, or reducing viral infections in a patient.

It is a further object of the invention to provide compounds and methods for treating, preventing, or reducing severe acute respiratory syndrome coronavirus 2 infection in a patient.

### SUMMARY OF THE INVENTION

Disclosed are compositions and methods for modulating neuropilin-1-mediated (NRP-1) signaling in a subject in need thereof. The methods involve administering to the subject an effective amount of a compound disclosed herein to reduce NRP-1 signaling in cells of the subject. In some forms, reducing NRP-signaling involves reducing binding of a peptide such as VEGF to NRP-1 or a co-receptor thereof. Preferably, the co-receptor is NRP-1/VEGFR2. In some forms, reducing NRP-1 signaling involves reducing phosphorylation of VEGFR2. Preferably, the phosphorylation of VEGFR2 involves phosphorylation of a tyrosine residue of VEGFR2, preferably an intracellular tyrosine residue.

Preferably, the compositions and methods are for treating, preventing, or reducing neuropathic pain or a viral infection in a patient. In some forms, the patient has or is likely to develop an infection from a virus. Preferably, the virus utilizes NRP-1 or a co-receptor to facilitate entry into cells. In some forms, the compositions contain a compound that treats, prevents, or reduces (i) neuropathic pain or (ii) infection by severe acute respiratory syndrome coronavirus 2 (SARS-CoV-2) in a patient.

The methods involve providing a formulation containing a compound or a pharmaceutically acceptable salt thereof, in an effective amount to treat, prevent, or reduce neuropathic pain in a patient. The amount can be effective to treat, prevent, or reduce one or more symptoms associated with neuropathic pain, such as allodynia, numbness; prickling or tingling; itching; sharp, jabbing, throbbing or burning pain; increased sensitivity to touch; lack of coordination and falling; muscle weakness; paralysis; heat intolerance; excessive sweating or not being able to sweat; bowel problems; bladder problems; digestive problems; changes in blood pressure causing dizziness or lightheadedness; or a combination thereof.

The methods also involve providing a formulation containing a compound or a pharmaceutically acceptable salt thereof, in an effective amount to treat, prevent, or reduce infection by SARS-CoV-2 in a patient.

The amount can be effective to treat, prevent, or reduce one or more symptoms associated with SARS-CoV-2, such as fever, congestion in the nasal sinuses and/or lungs, runny or stuffy nose, cough, sneezing, sore throat, body aches, fatigue, shortness of breath, chest tightness, or wheezing when exhaling, or a combination thereof.

The compound in the composition or formulation inhibits neuropilin receptor 1 signaling.

For treating, preventing, or reducing neuropathic pain, the compound has the structure:

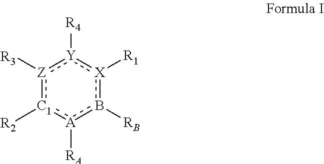

wherein:
C₁ is a carbon atom,
A, B, X, Y, and Z are independently carbon or nitrogen,
A, B, C₁, X, Y, and Z are bonded to none, one, or two hydrogen atoms according to valency,
the dashed lines in Formula I denote the presence or absence of a bond,
R₁, R_{B}, R_{A}, R₂, R₃, and R₄ are independently absent, hydrogen, oxo-(═O), hydroxyl, substituted alkyl, unsubstituted alkyl, substituted alkenyl, unsubstituted alkenyl, substituted aryl, unsubstituted aryl, substituted heteroaryl, unsubstituted heteroaryl, substituted amino, unsubstituted amino, substituted C₃-C₂₀ cyclyl, unsubstituted C₃-C₂₀ cyclyl, —NR₅R₆, —C(O)NR₉R₁₀, —C(O)R₁₁, carboxyl, or sulfonyl, or ZR₃ and YR₄ together form a substituted heteroaryl,
R₅ and R₆ are independently hydrogen, unsubstituted alkyl, substituted alkyl, unsubstituted aryl, substituted aryl, substituted heteroaryl, or unsubstituted heteroaryl,
R₉ and R₁₀ are independently hydrogen, substituted alkyl, or unsubstituted alkyl, or NR₉R₁₀ together form a substituted heterocyclyl, and
R₁₁ is hydroxyl.

Specific examples of compounds for treating, preventing, or reducing neuropathic pain include:

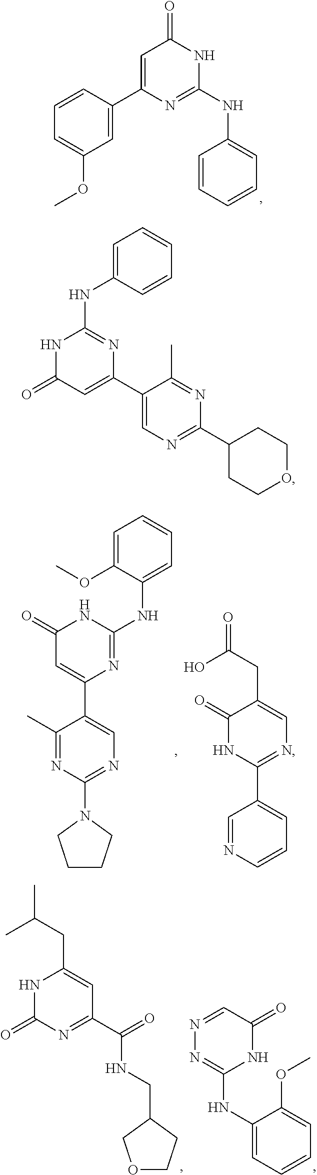

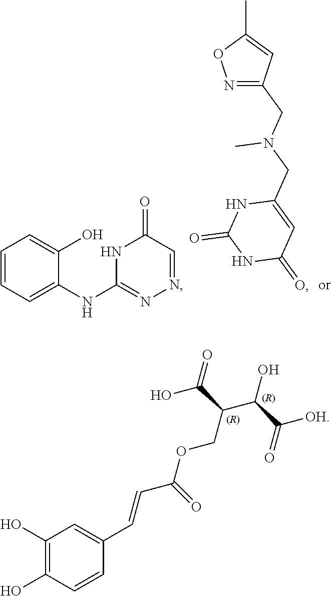

For treating, preventing, or reducing SARS-CoV-2, the compound has the structure of Formula I shown above, except that:
R₁, R_{B}, R_{A}, R₂, R₃, and R₄ are independently absent, hydrogen, oxo-(═O), hydroxyl, substituted alkyl (such as substituted C₁-C₁₀ alkyl, such as isobutyl), unsubstituted alkyl, substituted aryl, unsubstituted aryl, substituted heteroaryl (such as substituted with unsubstituted C₁-C₁₀ alkyl (such as methyl), unsubstituted heterocyclyl (such as pyrrolidin-1-yl), or both), unsubstituted heteroaryl, substituted C₃-C₂₀ cyclyl, unsubstituted C₃-C₂₀ cyclyl, —NR₅R₆, or —C(O)NR₉R₁₀,
R₅ and R₆ are independently hydrogen, unsubstituted aryl, substituted aryl, substituted heteroaryl, or unsubstituted heteroaryl,
R₉ and R₁₀ are independently hydrogen, substituted alkyl (such as heterocyclylalkyl), or unsubstituted alkyl, or NR₉R₁₀ together form a substituted heterocyclyl, and
at least one of XR₁, BR_{B}, AR_{A}, C₁R₂, ZR₃, and YR₄ is —C(O)— and an adjacent XR₁, BR_{B}, AR_{A}, C₁R₂, ZR₃, and YR₄ is —NH—.

Specific examples of compounds for treating, preventing, or reducing viral infection include:

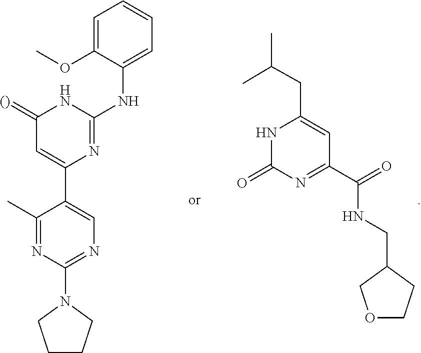

The compounds can be administered via one or more routes of administration. Exemplary routes of administration are topical, mucosal, transdermal, intradermal, intravenous, intramuscular, intraperitoneal, oral, intraocular, intranasal, intracranial, or a combination thereof.

### BRIEF DESCRIPTION OF THE DRAWINGS

FIGS. 1A, 1B, and 1C are schematics of NRP-1 domains, VEGF-A₁₆₅ binding, and CendR interaction network. FIG. 1A shows the domain architecture of NRP-1 with domain al from mouse (PDB ID 4gz9)(Janssen, et al., Nat. Struct. Mol. Biol. 2012, 19 (12), 1293-9), domains a2, b1, b2 (PDB ID 2qqm) (Appleton, et al., The EMBO journal 2007, 26 (23), 4902-12) and MAM (PDB ID 5L73)(Yelland and Djordjevic, Structure 2016, 24 (11), 2008-2015) from human. Bound Ca²⁺ shown as green spheres, missing loops as dashes, transmembrane domain as a rectangle. GAG indicates region of glycosaminoglycan modification. Asterisk (*) indicates VEGF-A interaction pocket. FIG. 1B shows a structure of NRP-1 b1 domain, with a white surface, in complex with the heparin binding domain (exon 7/8) of VEGF-A₁₆, shown as cartoon with exon 7, exon 8 (PDB ID 4deq) (Parker, et al., The Journal of Biological Chemistry 2012, 287 (14), 11082-9). FIG. 1C shows details of VEGF-A Glu154 and KPRR₁₆₄ interactions with NRP-1 (close up of FIG. 1B). Dashes indicate polar or salt-bridge contacts within 3.0 Å.

FIG. 2 shows the chemical structures of the top 20 hits. Compounds are grouped by common core motif. Molecules that adopt binding Mode II (9, 9a, 14, 17, 18, 16, 19, 20) have the atoms that form potential hydrogen bonds with Asp320, E319, or G318. An exemplary lactam core, 2(1H)-pyridone core, is highlighted with a gray box in structure 15.

FIG. 3 is a schematic showing an NRP-1 hydrophobic box. The hydrophobic groove formed by residues Tyr297, Tyr253, Trp301, and Thr316 are shown as gray spheres. Compound 1 is shown as sticks and contacts with hydrophobic box residues within 5 A shown as dashes. Additional binding site residues are also shown.

FIGS. 4A, 4B, and 4C are docked complexes showing hit compound poses in the NRP-1 b1-domain pocket. FIG. 4A shows overlay of hits that adopt binding Mode I. FIG. 4B shows overlay of hits that adopt binding Mode II. Polar interactions for representative compounds are shown as dashes for Mode I in FIG. 4C (compound 1) and FIG. 4D (compound 4) and for Mode II in FIG. 4E (compound 20) and FIG. 4F (compound 9).

FIG. 5 is a schematic showing structures of selected NRP-1 targeting compounds. Compound A (EG00229) from Jarvis, et al., J. Med. Chem. 2010, 53 (5), 2215-26, compound B (EGO1377) from Powell, et al., J. Med. Chem. 2018, 61 (9), 4135-4154, compound C is “compound 1” from Borriello, et al., Cancer Lett. 2014, 349 (2), 120-7, compound D is “bis-guanidinylated compound 32” from Novoa, et al., Bioorg. Med. Chem. 2010, 18 (9), 3285-98, compound E is ChemBridge ID: 7739526 from Starzec, et al., Bioorg. Med. Chem. 2014, 22 (15), 4042-8, and compound F is “NRPa-308” from Liu, et al., Cancer Lett. 2018, 414, 88-98.

FIG. 6 is a column graph showing the levels of pY1175 VEGFR2 normalized to the quantity of cells in each well. #p<0.05 compared to 0.1% DMSO (vehicle) treated cells without VEGF-A165. * p<0.05 compared to 0.1% DMSO+1 nM VEGF-A165 treated cells, Kruskal-Wallis test with Dunn's multiple test correction (n=7 replicates per condition). Data were analyzed by a repeated measures (RM) one-way analysis of variance (post hoc: Dunnett's), *p<0.05. The data were generated by screening of NRP-1/VEGF-A165 inhibitors by in-cell Western. Cathecholamine A differentiated (CAD) cells were grown in 96 well plates. Cells were treated with the NRP-1/VEGF-A165 inhibitors at 12.5 μM or SARS-CoV-2 Spike (100 nM) in combination with 1 nM VEGF-A165 as indicated. Cells were stained for pY1175 VEGFR2 as a marker of the activation of the pathway by VEGF-A165. Phosphorylated VEGFR2 was increased by the addition of 1 nM VEGF-A165 on the cells.

FIG. 7 is a column graph showing screening for VSV-eGFP-SARS-CoV-2 inhibition. Compounds were screened at 25 μM for inhibition of VSV-eGFP-SARS-CoV-2 infection of Vero-E6-TMPRSS2 cells. Recombinant Spike S1 domain was included at 68 nM. Cells were infected for 36 h prior to live cell automated microscopy and quantification of sum GFP fluorescence intensity, normalized to cell count by HCS CellMask Blue, was measured and for each well and plotted with Prism 6. Results are presented as mean intensity ±SEM, #P<0.05 vs. mock; *P<0.05 versus DMSO (n=3 replicates). Data was analyzed by a one-way analysis of variance (post hoc: Dunnett's), *p<0.05.

FIG. 8 is a line graph showing antagonism of NRP-1/VEGF-A signaling using compound 4. As shown, compound 4 reverses spared nerve injury (SNI)-induced mechanical allodynia. SNI elicited mechanical allodynia 21 days after surgery. Paw withdrawal thresholds for SNI rats (Sprague Dawley, male) administered saline (vehicle) or NRP-1 inhibitor (2.14 mg/5 ml) intrathecally (i.t.); n=5). P values, versus control, are indicated. Data are shown as mean±s.e.m. and were analyzed by non-parametric two-way analysis of variance where time was the within subject factor and treatment was the between subject factor (post hoc: Sidak). The experiments were analyzed by an investigator blinded to the treatment.

FIG. 9 is a schematic of pharmacophore models built from ligand-receptor complexes described herein. Thin lines indicate bond distances between pharmacophores. Two binding modes (Mode I and Mode II) are also shown. Pharmacophore features are labeled as hydrogen bond acceptors (acceptors), hydrogen bond donors (donors), and aromatic rings. The neighboring or contact protein residues are listed.

FIG. 10 shows chemical structures of analogs identified for series 1, 2, and 4 shown in FIG. 2. Analogs in gray rectangles indicate scaffold similarity and series membership. Compound identifiers refer to Zinc ID or E-molecules ID.

FIG. 11 is a line graph of representative concentration-response curves of inhibition of NRP-1NEGF-A interaction assessed by an enzyme-linked immunosorbent assay for the compounds described herein.

### DETAILED DESCRIPTION OF THE INVENTION

### I. Definitions

“Acyl” refers to the group R—C(O)—, where R is unsubstituted alkyl (e.g., unsubstituted C₁-C₁₀ alkyl), substituted alkyl (e.g. substituted C₁-C₁₀ alkyl), unsubstituted aryl, substituted aryl, unsubstituted heteroaryl, substituted heteroaryl, unsubstituted C₃-C₂₀ cyclyl, substituted C₃-C₂₀ cyclyl, unsubstituted heterocyclyl, or substituted heterocyclyl. In its simplest form, R is unsubstituted C₁-C₁₀ alkyl (e.g. unsubstituted C₁ alkyl, unsubstituted C₂ alkyl, unsubstituted C₃ alkyl, unsubstituted C₄ alkyl, unsubstituted C₅ alkyl, unsubstituted C₆ alkyl, unsubstituted C₇ alkyl, unsubstituted C₅ alkyl, unsubstituted C₉ alkyl, or unsubstituted C₁₀ alkyl), or substituted C₁-C₁₀ alkyl (e.g. substituted C₁ alkyl, substituted C₂ alkyl, substituted C₃ alkyl, substituted C₄ alkyl, substituted C₅ alkyl, substituted C₆ alkyl, substituted C₇ alkyl, substituted C₈ alkyl, substituted C₉ alkyl, or substituted C₁₀ alkyl).

“Arylalkyl” or “aralkyl” refers to an alkyl group that is substituted with a substituted or unsubstituted aryl or heteroaryl group.

“Heterocyclylalkyl” refers to an alkyl group that is substituted with a substituted or unsubstituted heterocyclyl group.

The term “sulfonyl” is represented by the formula

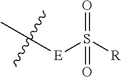

wherein E is absent, or E is alkyl, alkenyl, alkynyl, aralkyl, alkylaryl, cycloalkyl, aryl, heteroaryl, heterocyclyl, wherein independently of E, R represents a hydrogen, substituted or unsubstituted alkyl, substituted or unsubstituted alkenyl, substituted or unsubstituted alkynyl, substituted or unsubstituted amine, substituted or unsubstituted cycloalkyl, substituted or unsubstituted heterocyclyl, substituted or unsubstituted alkylaryl, substituted or unsubstituted arylalkyl, substituted or unsubstituted aryl, or substituted or unsubstituted heteroaryl, —(CH₂)ₘ—R′″, or E and R taken together with the S atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of E and R can be substituted or unsubstituted amine, to form a “sulfonamide” or “sulfonamido.”

The term “substituted sulfonyl” represents a sulfonyl in which E and R are independently substituted. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof.

### II. Compositions

Disclosed are compositions and methods for treating, preventing, or reducing neuropathic pain or a viral infection in a patient. For example, the compositions may inhibit, and/or methods may involve inhibiting, neuropilin receptor 1 signaling.

A. Neuropathic Pain

The disclosed methods include treating, preventing, or reducing neuropathic pain in a patient, involving providing a formulation containing a compound or a pharmaceutically acceptable salt thereof, in an effective amount to treat, prevent, or reduce the neuropathic pain.

In some forms of the methods, the compound has the structure:

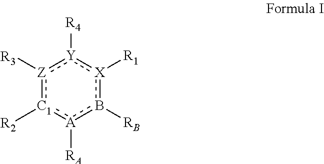

wherein:
C₁ is a carbon atom,
A, B, X, Y, and Z are independently carbon or nitrogen,
A, B, C₁, X, Y, and Z are bonded to none, one, or two hydrogen atoms according to valency,
the dashed lines in Formula I denote the presence or absence of a bond,
R₁, R_{B}, R_{A}, R₂, R₃, and R₄ are independently absent, hydrogen, oxo-(═O), hydroxyl, substituted alkyl, unsubstituted alkyl, substituted alkenyl, unsubstituted alkenyl, substituted aryl, unsubstituted aryl, substituted heteroaryl, unsubstituted heteroaryl, substituted amino, unsubstituted amino, substituted C₃-C₂₀ cyclyl, unsubstituted C₃-C₂₀ cyclyl, —NR₅R₆, —C(O)NR₉R₁₀, —C(O)R₁₁, carboxyl, or sulfonyl, or ZR₃ and YR₄ together form a substituted heteroaryl,
R₅ and R₆ are independently hydrogen, unsubstituted alkyl, substituted alkyl, unsubstituted aryl, substituted aryl, substituted heteroaryl, or unsubstituted heteroaryl,
R₉ and R₁₀ are independently hydrogen, substituted alkyl, or unsubstituted alkyl, or NR₉R₁₀ together form a substituted heterocyclyl,
R₁₁ is hydroxyl, —SH, —NH₂, halogen, —CN, —OCN, —CNO, —NO₂, or —COOH. In some forms, R₁₁ is hydroxyl.

In some forms of the methods, the following moiety of Formula I:

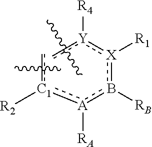

has the structure

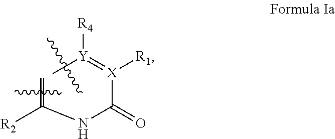

wherein
for Formula Ia, X is carbon, and Y is carbon or nitrogen,
1) R₁ is hydrogen, substituted alkyl (such as substituted C₁-C₁₀ alkyl, such as substituted C₁ alkyl, preferably directly bonded to carboxyl group), or unsubstituted alkyl,
R₂ is —NR₅R₆; unsubstituted heteroaryl; substituted heteroaryl; unsubstituted aryl; substituted aryl [such as substituted with unsubstituted C₁-C₁₀ alkoxy (such as methoxy), unsubstituted C₁-C₁₀ alkyl (such as ethyl), hydroxyl, halogen (F, Cl, Br, I), unsubstituted anilinyl, unsubstituted aroxy, unsubstituted heterocyclyl (such as 4-morpholinyl), carboxyl, acyl, cyano-, —SH, nitro-, —NR₇R₈, etc. or a combination thereof, wherein R₇ and R₈ are independently hydrogen, unsubstituted alkyl, unsubstituted aryl, unsubstituted heteroaryl, unsubstituted C₃-C₂₀ cyclyl, unsubstituted heterocyclyl, or unsubstituted alkyl]; unsubstituted C₃-C₂₀ cyclyl (such as unsubstituted C₃ cyclyl such as cyclopropyl); substituted C₃-C₂₀ cyclyl; unsubstituted heterocyclyl; substituted heterocyclyl (such as substituted piperidinyl (e.g., 1-methyl-5-hydroxymethylpiperidin-3-yl)); substituted alkyl [such as substituted C₁-C₁₀ alkyl (such as substituted C₁-C₃ alkyl, e.g., isopropyl); substituted C₁ alkyl, preferably directly bonded to a secondary amino group containing unsubstituted heteroaryl, substituted heteroaryl, unsubstituted aryl, substituted aryl, unsubstituted heterocyclyl, substituted heterocyclyl (e.g. pyrrolidin-2-one-5-yl), preferably containing unsubstituted heteroaryl and substituted heterocyclyl]; or unsubstituted alkyl,
wherein R₅ and R₆ are independently hydrogen; unsubstituted aryl; substituted aryl [such as substituted with unsubstituted C₁-C₁₀ alkoxy (such as methoxy), unsubstituted C₁-C₁₀ alkyl (such as ethyl), hydroxyl, halogen (F, Cl, Br, I), unsubstituted anilinyl, unsubstituted aroxy, unsubstituted heterocyclyl (such as 4-morpholinyl), carboxyl, acyl, cyano-, —SH, nitro-, —NR₇R₈, etc. or a combination thereof, wherein R₇ and R₈ are independently hydrogen, unsubstituted alkyl, unsubstituted aryl, unsubstituted heteroaryl, unsubstituted C₃-C₂₀ cyclyl, unsubstituted heterocyclyl, or unsubstituted alkyl]; unsubstituted heteroaryl; substituted heteroaryl [such as substituted with —NR₇R₈, hydroxyl, halogen, cyano-, —SH, nitro-, unsubstituted alkoxy, carboxyl, etc, or a combination thereof, wherein R₇ and R₈ are independently hydrogen, unsubstituted alkyl, unsubstituted aryl, unsubstituted heteroaryl, unsubstituted C₃-C₂₀ cyclyl, unsubstituted heterocyclyl, or unsubstituted alkyl, preferably wherein R₇ and R₈ are both hydrogen]; unsubstituted C₃-C₂₀ cyclyl; substituted C₃-C₂₀ cyclyl; unsubstituted heterocyclyl; substituted heterocyclyl; unsubstituted alkyl, or substituted alkyl, preferably wherein at least one of R₅ and R₆ is unsubstituted aryl; substituted aryl; unsubstituted heteroaryl; substituted heteroaryl; unsubstituted C₃-C₂₀ cyclyl; substituted C₃-C₂₀ cyclyl; unsubstituted heterocyclyl; or substituted heterocyclyl,
R₄ is absent, hydrogen, substituted aryl [such as substituted with unsubstituted C₁-C₁₀ alkoxy (such as methoxy), unsubstituted C₁-C₁₀ alkyl (such as methyl), or a combination thereof], unsubstituted aryl (such as phenyl), substituted heteroaryl [such as substituted withs unsubstituted C₁-C₁₀ alkyl (such as methyl), unsubstituted C₁-C₁₀ alkoxy (such as methoxy), unsubstituted heterocyclyl (such as 4-morpholinyl, 1-pyrrolidinyl), or a combination thereof, such as unsubstituted C₁-C₁₀ alkyl (such as methyl) and unsubstituted heterocyclyl (such as 4-morpholinyl), or unsubstituted C₁-C₁₀ alkyl (such as methyl) and unsubstituted heterocyclyl (such as 1-pyrrolidinyl)], unsubstituted heteroaryl [such as pyridinyl, such as pyridin-3yl], unsubstituted alkyl [such as unsubstituted C₁-C₁₀ alkyl (such as methyl)], substituted alkyl, or —NR₅R₆, wherein R₅ and R₆ are independently hydrogen; unsubstituted aryl; substituted aryl [such as substituted with unsubstituted C₁-C₁₀ alkoxy (such as methoxy), unsubstituted C₁-C₁₀ alkyl (such as ethyl), hydroxyl, halogen (F, Cl, Br, I), unsubstituted anilinyl, unsubstituted aroxy, unsubstituted heterocyclyl (such as 4-morpholinyl), carboxyl, acyl, cyano-, —SH, nitro-, —NR₇R₈, etc. or a combination thereof, wherein R₇ and R₈ are independently hydrogen, unsubstituted alkyl, unsubstituted aryl, unsubstituted heteroaryl, unsubstituted C₃-C₂₀ cyclyl, unsubstituted heterocyclyl, or unsubstituted alkyl]; unsubstituted heteroaryl; substituted heteroaryl [such as substituted with —NR₇R₈, hydroxyl, halogen, cyano-, —SH, nitro-, unsubstituted alkoxy, carboxyl, etc, wherein R₇ and R₈ are independently hydrogen, unsubstituted alkyl, unsubstituted aryl, unsubstituted heteroaryl, unsubstituted C₃-C₂₀ cyclyl, unsubstituted heterocyclyl, or unsubstituted alkyl],
or
2) X—R₁ and Y—R₄ together form an unsubstituted heteroaryl [such as thiophene], or substituted heteroaryl.

In some forms of the methods, the following moiety of Formula I:

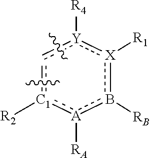

has the structure

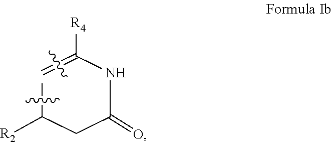

wherein
R₂ is —NR₅R₆ wherein R₅ and R₆ are independently hydrogen; unsubstituted aryl; substituted aryl; unsubstituted heteroaryl; substituted heteroaryl [such as substituted with —NR₇R₈, hydroxyl, halogen, cyano-, —SH, nitro-, unsubstituted alkoxy, etc, or a combination thereof, wherein R₇ and R₈ are independently hydrogen, unsubstituted alkyl, unsubstituted aryl, unsubstituted heteroaryl, unsubstituted C₃-C₂₀ cyclyl, unsubstituted heterocyclyl, or unsubstituted alkyl, preferably wherein R₇ and R₈ are both hydrogen]; unsubstituted C₃-C₂₀ cyclyl; substituted C₃-C₂₀ cyclyl; unsubstituted heterocyclyl; substituted heterocyclyl; unsubstituted alkyl, or substituted alkyl, preferably wherein at least one of R₅ and R₆ is unsubstituted aryl; substituted aryl; unsubstituted heteroaryl; substituted heteroaryl; unsubstituted C₃-C₂₀ cyclyl; substituted C₃-C₂₀ cyclyl; unsubstituted heterocyclyl; or substituted heterocyclyl,
R₄ is hydrogen, substituted aryl [such as substituted with unsubstituted C₁-C₁₀ alkoxy (such as methoxy)], unsubstituted aryl, or substituted heteroaryl [such as substituted with —NR₇R₈, hydroxyl, halogen, cyano-, —SH, nitro-, unsubstituted alkoxy, etc, or a combination thereof, wherein R₇ and R₈ are independently hydrogen, unsubstituted alkyl, unsubstituted aryl, unsubstituted heteroaryl, unsubstituted C₃-C₂₀ cyclyl, unsubstituted heterocyclyl, or unsubstituted alkyl].

In some forms of the methods, Z is carbon, and for Formula Ia, Y is carbon, X is carbon or nitrogen, and
R₂ is substituted alkyl [such as substituted C₁-C₁₀ alkyl, such as isobutyl, 2-methyl-3-hydroxypropyl, etc.], unsubstituted alkyl [such as unsubstituted C₁-C₁₀ alkyl, such as methyl, ethyl, etc.], —NR₅R₆, wherein R₅ and R₆ are independently hydrogen; substituted heteroaryl [such as pyridin-4-yl substituted with —NR₇R₈, hydroxyl, halogen, cyano-, —SH, nitro-, unsubstituted alkoxy, etc, or a combination thereof, wherein R₇ and R₈ are independently hydrogen, unsubstituted alkyl, unsubstituted aryl, unsubstituted heteroaryl, unsubstituted C₃-C₂₀ cyclyl, unsubstituted heterocyclyl, or unsubstituted alkyl, preferably wherein R₇ and R₈ are both hydrogen], unsubstituted heteroaryl, substituted aryl, unsubstituted aryl, substituted C₃-C₂₀ cyclyl, unsubstituted C₃-C₂₀ cyclyl, substituted heterocyclyl, or unsubstituted heterocyclyl,
R₄ is —C(O)NR₉R₁₀, —C(O)R₁₁, substituted heteroaryl, unsubstituted heteroaryl, or substituted aryl [such as substituted with unsubstituted C₁-C₁₀ alkoxy (such as methoxy)],
wherein R₉ and R₁₀ are independently hydrogen, substituted alkyl (such as heterocyclylalkyl, arylalkyl, 2-methoxyethyl, 2-hydroxyethyl, 2-(N-methylsulfonamido)ethyl, 1-carboxy-1-cyclopropylmethyl), or unsubstituted alkyl (such as methyl), or NR₉R₁₀ together form a substituted heterocyclyl, substituted heterocyclyl fused with heteroaryl,
R₁₁ is hydroxyl, —SH, —NH₂, halogen, —CN, —OCN, —CNO, —NO₂, or —COOH. In some forms, R₁₁ is hydroxyl.

In some forms of the methods, the following moiety of Formula I

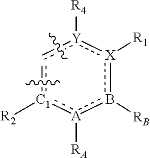

has the structure

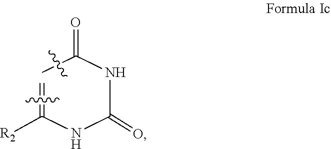

wherein
R₂ is substituted alkyl [such as substituted C₁-C₁₀ alkyl (such as substituted C₁ alkyl, preferably directly bonded to a carboxyl group or —NR₅ₐR₆ₐ, wherein R₅ₐ and R₆ₐ are independently hydrogen; unsubstituted C₁-C₁₀ alkyl (such as methyl); arylalkyl preferably substituted with —NH₂ or methyl; unsubstituted C₃-C₂₀ cyclyl (such as unsubstituted C₅ cyclyl such as unsubstituted cyclopentyl); substituted aryl such as phenyl substituted with sulfonamide], carboxyl, sulfonyl (such as unsubstituted alkyl-S(O)₂-substituted alkyl/alkylene such as CH₃S(O)₂—CH₂—), or —NR₅R₆, wherein R₅ and R₆ are independently hydrogen; substituted aryl [such as phenyl substituted with hydroxyl, halogen, cyano-, —SH, nitro-, unsubstituted alkoxy, etc, or a combination thereof, substituted with —NR₇R₈, wherein R₇ and R₈ are independently hydrogen, unsubstituted alkyl, unsubstituted aryl, unsubstituted heteroaryl, unsubstituted C₃-C₂₀ cyclyl, unsubstituted heterocyclyl, or unsubstituted alkyl], unsubstituted aryl, unsubstituted heteroaryl, substituted heteroaryl, substituted C₃-C₂₀ cyclyl, unsubstituted C₃-C₂₀ cyclyl, substituted heterocyclyl, or unsubstituted heterocyclyl.

In some forms of the methods, Formula I has the structure

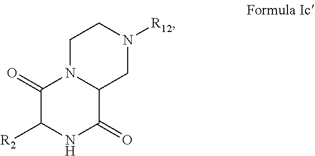

wherein
R₂ is substituted alkyl (such as arylalkyl), or unsubstituted alkyl,
R₁₂ is —C(O)R₁₂ wherein R₁₂ is substituted alkyl [such as substituted C₁-C₁₀ alkyl (such as substituted C₁ alkyl containing unsubstituted alkoxy (e.g., methoxy))].

In some forms of the methods, Formula I has the structure:

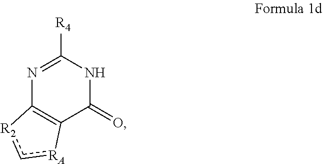

wherein
R₂ and R_{A} are nitrogen bonded to none or one hydrogen atom according to valency, and the dashed lines in Formula id denote the presence or absence of a bond,
R₄ is —NR₅R₆, wherein R₅ and R₆ are independently hydrogen, substituted alkyl [such as substituted C₁-C₁₀ alkyl (such as substituted C₁ alkyl, preferably directly bonded to carboxyl group)], or unsubstituted alkyl.

In some forms of the methods, Formula I has the structure:

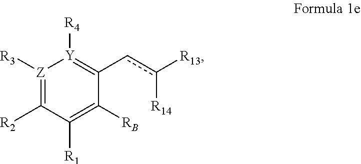

wherein for Formula 1e,
the dashed line denotes the presence or absence of a bond,
Y and Z are carbon,
R₂ and R₃ are hydroxyl, or ZR₃ and YR₄ together form a substituted heteroaryl ring preferably substituted with a substituted aryl such as 3,4-dihydroxyphenyl,
R₄, R_{A}, and R_{B} are independently hydrogen, hydroxyl, unsubstituted alkyl, substituted alkyl, unsubstituted aryl, substituted aryl, unsubstituted heteroaryl, or substituted heteroaryl, preferably R₄, R_{A}, and R_{B} are hydrogen when R₂ and R₃ are hydroxyl, and preferably R_{A} is hydroxy when ZR₃ and YR₄ together form a substituted heteroaryl ring,
R₁₃ and R₁₄ are independently hydrogen, —C(O)R₁₅, or substituted alkenyl [such as substituted C₂-C₁₀ alkenyl substituted with a carboxyl, substituted aryl (3,4-dihydroxyphenyl), or preferably both], preferably wherein when the dashed line in Formula 1e denotes the presence of a bond, one of R₁₃ or R₁₄ is hydrogen,
wherein R₁₅ is substituted alkyl [such as C₁-C₁₀ substituted alkyl such as substituted C₃ alkyl substituted with carboxyl, substituted aryl (3,4-dihydroxyphenyl), or preferably both, such as substituted C₂ alkyl substituted with hydroxyl, carboxyl, or preferably both], substituted alkoxy [such as substituted C₁-C₁₀ alkoxy such as substituted C₃ alkoxy substituted with hydroxyl, carboxyl, unsubstituted alkyl (methyl), or preferably hydroxyl, carboxyl, unsubstituted alkyl (methyl), preferably hydroxyl and carboxyl, such as substituted C₂ alkoxy substituted with hydroxyl, carboxyl, substituted aryl (such as 3,4-dihydroxyphenyl), —C(O)OR₁₆ wherein R₁₆ is unsubstituted alkyl (such as methyl), or preferably hydroxyl and carboxyl, carboxyl and substituted aryl (such as 3,4-dihydroxyphenyl), —C(O)OR₁₆ and substituted aryl (such as 3,4-dihydroxyphenyl)], substituted aroxy [such as 2,3-dihydroxyphenyl], or substituted heterocyclyl [such as 2,3-dihydro-2H-pyran-4-yl substituted with hydroxyl, carboxyl, or preferably both].

In some forms of the methods, Formula I and Formula Ia are as described above, except that Z is nitrogen, R₃ is absent, and for Formula Ia:
X is carbon and Y is carbon,
R₁ is hydrogen or substituted C₁-C₁₀ alkyl (such as substituted C₁ alkyl, preferably directly bonded to carboxyl group),
R₂ is —NR₅R₆, unsubstituted heteroaryl, unsubstituted C₃-C₂₀ cyclyl (such as unsubstituted C₃ cyclyl such as cyclopropyl), substituted heterocyclyl, substituted C₁-C₁₀ alkyl (such as substituted C₁-C₃ alkyl, e.g., isopropyl; substituted C₁ alkyl, preferably directly bonded to a secondary amino group containing unsubstituted heteroaryl, or substituted heterocyclyl (e.g. pyrrolidin-2-one-5-yl), preferably containing unsubstituted heteroaryl and substituted heterocyclyl), substituted aryl [such as substituted with unsubstituted C₁-C₁₀ alkoxy (such as methoxy)], wherein R₅ and R₆ are independently hydrogen, unsubstituted aryl, substituted aryl (such as substituted with unsubstituted C₁-C₁₀ alkoxy (such as methoxy), hydroxyl, halogen, unsubstituted anilinyl, unsubstituted aroxy, unsubstituted heterocyclyl (such as 4-morpholinyl), carboxyl, acyl), unsubstituted heteroaryl, or substituted heteroaryl (such as substituted with carboxyl, or —NR₇R₈, preferably wherein R₇ and R₈ are both hydrogen),
R₄ is hydrogen, substituted aryl (such as substituted with unsubstituted C₁-C₁₀ alkoxy (such as methoxy), unsubstituted C₁-C₁₀ alkyl (such as methyl), or a combination thereof), unsubstituted aryl (such as phenyl), substituted heteroaryl (such as substituted with unsubstituted C₁-C₁₀ alkyl (such as methyl), unsubstituted C₁-C₁₀ alkoxy (such as methoxy), unsubstituted heterocyclyl (such as 4-morpholinyl, 1-pyrrolidinyl), or a combination thereof, such as unsubstituted C₁-C₁₀ alkyl (such as methyl) and unsubstituted heterocyclyl (such as 4-morpholinyl), or unsubstituted C₁-C₁₀ alkyl (such as methyl) and unsubstituted heterocyclyl (such as 1-pyrrolidinyl), unsubstituted heteroaryl (such as pyridinyl, such as pyridin-3yl), unsubstituted C₁-C₁₀ alkyl (such as methyl), or —NR₅R₆, wherein R₅ and R₆ are independently hydrogen, or substituted heteroaryl [such as substituted with carboxyl], or
X—R₁ and Y—R₄ together form an unsubstituted heteroaryl (such as thiophene) and preferably R₂ is a substituted C₁-C₁₀ alkyl such as substituted C₁-C₃ alkyl, e.g., isopropyl; substituted C₁ alkyl, preferably directly bonded to a secondary amino group containing unsubstituted heteroaryl, substituted heterocyclyl (e.g., pyrrolidin-2-one-5-yl), preferably containing unsubstituted heteroaryl and substituted heterocyclyl.

In some forms of the methods, Formula I and Formula Ia are as described above, except that Z is nitrogen, R₃ is absent, and for Formula Ia:
X is carbon and Y is carbon,
R₂ is —NR₅R₆, substituted aryl (such as substituted with unsubstituted C₁-C₁₀ alkoxy (such as methoxy), or unsubstituted heteroaryl, and R₅ and R₆ are independently hydrogen, unsubstituted aryl, or substituted aryl (such as substituted with unsubstituted C₁-C₁₀ alkoxy (such as methoxy)), or substituted heteroaryl (such as substituted with carboxyl or —NH₂), and
R₄ is hydrogen, substituted aryl (such as substituted with unsubstituted C₁-C₁₀ alkoxy (such as methoxy), substituted heteroaryl (such as substituted with unsubstituted C₁-C₁₀ alkyl (such as methyl), unsubstituted C₁-C₁₀ alkoxy (such as methoxy), unsubstituted heterocyclyl (such as 4-morpholinyl, 1-pyrrolidinyl), or a combination thereof, such as unsubstituted C₁-C₁₀ alkyl (such as methyl) and unsubstituted heterocyclyl (such as 4-morpholinyl), or unsubstituted C₁-C₁₀ alkyl (such as methyl) and unsubstituted heterocyclyl (such as 1-pyrrolidinyl), or —NR₅R₆, wherein R₅ and R₆ are independently hydrogen, or substituted heteroaryl [such as substituted with carboxyl].

In some forms of the methods, Formula I and Formula Ia are as described above, except that Z is nitrogen, R₃ is absent, and for Formula Ia:
X is carbon and Y is carbon,
R₁ is hydrogen,
R₂ is —NR₅R₆, or substituted aryl (such as substituted with unsubstituted C₁-C₁₀ alkoxy (such as methoxy)), wherein R₅ and R₆ are independently hydrogen, unsubstituted aryl, substituted aryl (such as substituted with unsubstituted C₁-C₁₀ alkoxy (such as methoxy), hydroxyl, halogen, unsubstituted anilinyl, unsubstituted aroxy, unsubstituted heterocyclyl (such as 4-morpholinyl), carboxyl, acyl), unsubstituted heteroaryl, or substituted heteroaryl (such as substituted with carboxyl or —NR₇R₈, preferably wherein R₇ and R₈ are both hydrogen), and wherein one of R₅ and R₆ is hydrogen, and
R₄ is substituted aryl (such as substituted with unsubstituted C₁-C₁₀ alkoxy (such as methoxy), unsubstituted C₁-C₁₀ alkyl (such as methyl), or a combination thereof), unsubstituted aryl, substituted heteroaryl (such as substituted with unsubstituted C₁-C₁₀ alkyl (such as methyl), unsubstituted C₁-C₁₀ alkoxy (such as methoxy), unsubstituted heterocyclyl (such as 4-morpholinyl, 1-pyrrolidinyl), or a combination thereof, such as unsubstituted C₁-C₁₀ alkyl (such as methyl) and unsubstituted heterocyclyl (such as 4-morpholinyl), unsubstituted heteroaryl, or —NR₅R₆, (##1g′##) wherein R₅ and R₆ are independently hydrogen, or substituted heteroaryl [such as substituted with carboxyl].

In some forms of the methods, Formula I and Formula Ib are as described above, except that Z is nitrogen, R₃ is absent, and for Formula Ib:
R₂ is —NR₅R₆, wherein R₅ and R₆ are independently hydrogen; unsubstituted aryl; substituted heteroaryl (such as substituted with —NH₂), wherein at least one of R₅ and R₆ is unsubstituted aryl; or substituted heteroaryl (such as substituted with —NH₂), and
R₄ is substituted aryl (such as substituted with unsubstituted C₁-C₁₀ alkoxy (such as methoxy)).

In some forms of the methods, Formula I and Formula Ia are as described above, except that for Formula Ia:
Y is nitrogen and R₄ is absent,
R₁ is hydrogen, and
R₂ is —NR₅R₆, wherein R₅ and R₆ are independently hydrogen or substituted aryl (such as substituted with unsubstituted C₁-C₁₀ alkoxy (such as methoxy), unsubstituted C₁-C₁₀ alkyl (such as ethyl), or hydroxyl).

In some forms of the methods, for Formula I, Z is carbon,
R₂ is substituted C₁-C₁₀ alkyl (such as isobutyl, 2-methyl-3-hydroxypropyl], unsubstituted C₁-C₁₀ alkyl (such as methyl, ethyl), —NR₅R₆, wherein R₅ and R₆ are independently hydrogen; unsubstituted aryl, or substituted heteroaryl (such as pyridin-4-yl substituted with —NH₂),
R₄ is —C(O)NR₉R₁₀, —C(O)R₁₁, substituted heteroaryl, or unsubstituted heteroaryl, or substituted aryl (such as substituted with unsubstituted C₁-C₁₀ alkoxy (such as methoxy)),
wherein R₉ and R₁₀ are independently hydrogen, substituted alkyl (such as heterocyclylalkyl, arylalkyl, 2-methoxyethyl, 2-hydroxyethyl, 2-(N-methylsulfonamido)ethyl, 1-carboxy-1-cyclopropylmethyl), or unsubstituted alkyl (such as methyl), or NR₉R₁₀ together form a substituted heterocyclyl (such as substituted with unsubstituted heteroaryl, substituted heteroaryl that is substituted with unsubstituted C₁-C₁₀ alkyl, substituted C₁-C₁₀ alkoxy that is substituted with unsubstituted heteroaryl, unsubstituted C₁-C₁₀ alkyl, substituted aryl that is substituted with halogen), unsubstituted heterocyclyl (fused with unsubstituted heteroaryl or substituted heteroaryl that is substituted with 3-methoxyphenyl), or hydroxyl), and
R₁₁ is hydroxyl, —SH, —NH₂, halogen, —CN, —OCN, —CNO, —NO₂, or —COOH, preferably, R₁₁ is hydroxyl.

In some forms of the methods, for Formula I, Z is carbon,
R₂ is substituted C₁-C₁₀ alkyl (such as isobutyl, 2-methyl-3-hydroxypropyl), —NR₅R₆, wherein R₅ and R₆ are independently hydrogen; substituted heteroaryl (such as pyridin-4-yl substituted with —NH₂), or unsubstituted aryl,
R₄ is —C(O)NR₉R₁₀, substituted heteroaryl (such as substituted with unsubstituted C₁-C₁₀ alkyl such as methyl), or unsubstituted heteroaryl, or substituted aryl (such as substituted with unsubstituted C₁-C₁₀ alkoxy (such as methoxy)), and
wherein R₉ and R₁₀ are independently hydrogen, or substituted alkyl (such as 2-hydroxyethyl), or NR₉R₁₀ together form a substituted heterocyclyl (such as substituted with hydroxyl).

In some forms of the methods, for Formula I, Z is carbon,
R₂ is substituted C₁-C₁₀ alkyl (such as isobutyl), or —NR₅R₆, wherein R₅ and R₆ are independently hydrogen or unsubstituted aryl,
R₄ is —C(O)NR₉R₁₀, or substituted aryl (such as substituted with unsubstituted C₁-C₁₀ alkoxy (such as methoxy)), and
R₉ and R₁₀ are independently hydrogen or substituted alkyl (such as heterocyclylalkyl).

In some forms of the methods, for Formula I, Z is carbon, R₃ is hydrogen or hydroxyl, and for Formula Ic:
R₂ is substituted C₁-C₁₀ alkyl (such as substituted C₁ alkyl, preferably directly bonded to a carboxyl group or —NR₅ₐR₆ₐ, wherein R₅ₐ and R₆ₐ are independently hydrogen; unsubstituted C₁-C₁₀ alkyl (such as methyl); arylalkyl preferably substituted with —NH₂ or methyl; unsubstituted C₃-C₂₀ cyclyl (such as unsubstituted C₅ cyclyl such as unsubstituted cyclopentyl); substituted aryl such as phenyl substituted with sulfonamide), carboxyl, sulfonyl (such as unsubstituted alkyl-S(O)₂-substituted alkyl/alkylene such as CH₃S(O)₂—CH₂—), or —NR₅R₆,
wherein R₅ and R₆ are independently hydrogen; or substituted aryl [such as phenyl substituted with hydroxyl].

In some forms of the methods, for Formula I, Z is carbon, R₃ is hydrogen or hydroxyl, and for Formula Ic:
R₂ is substituted C₁-C₁₀ alkyl (such as substituted C₁ alkyl directly bonded to —NR₅ₐR₆ₐ, wherein R₅ₐ and R₆ₐ are independently unsubstituted C₁-C₁₀ alkyl (such as methyl), arylalkyl substituted with —NH₂, or both; —NR₅R₆,
wherein R₅ and R₆ are independently hydrogen; substituted aryl (such as phenyl substituted with hydroxyl).

In some forms of the methods, for Formula I, Z is carbon, R₃ is hydrogen or hydroxyl, and for Formula Ic:
R₂ is substituted C₁-C₁₀ alkyl (such as substituted C₁ alkyl, directly bonded to —NR₅ₐR_{6aQ}, wherein R₅ₐ and R₆ₐ are independently unsubstituted C₁-C₁₀ alkyl (such as methyl) or arylalkyl substituted with methyl).

In some forms of the methods, for Formula I, Z is carbon, R₃ is hydrogen or hydroxyl, and for Formula Ic, R₃ is hydrogen.

In some forms of the methods, Formula I has the structure

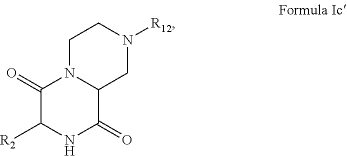

wherein
R₂ is arylalkyl,
R₁₂ is —C(O)R₁₂ wherein R₁₂ is substituted C₁-C₁₀ alkyl (such as substituted C₁ alkyl directly bonded to unsubstituted C₁-C₁₀ alkoxy (e.g. methoxy)).

In some forms of the methods, Formula I has the structure:

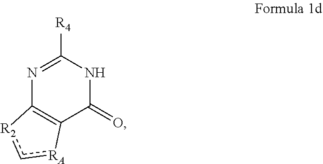

wherein
R₂ and R_{A} are nitrogen bonded to none or one hydrogen atom according to valency, and the dashed lines in Formula id denote the presence or absence of a bond, and
R₄ is —NR₅R₆, wherein R₅ and R₆ are independently hydrogen, or substituted C₁-C₁₀ alkyl (such as substituted C₁ alkyl, preferably directly bonded to carboxyl group).

In some forms of the methods, Formula I has the structure:

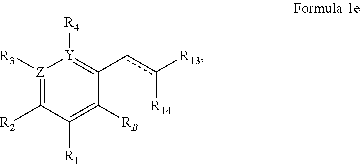

wherein for Formula 1e,
the dashed line denotes the presence or absence of a bond,
Y and Z are carbon,
R₂ and R₃ are hydroxyl, or ZR₃ and YR₄ together form a substituted heteroaryl ring substituted with a substituted aryl such as 3,4-dihydroxyphenyl,
R₄, R_{A}, and R_{B} are independently hydrogen, or hydroxyl, preferably R₄, R_{A}, and R_{B} are hydrogen when R₂ and R₃ are hydroxyl, and preferably R_{A} is hydroxy when ZR₃ and YR₄ together form a substituted heteroaryl ring,
R₁₃ and R₁₄ are independently hydrogen, —C(O)R₁₅, or substituted C₂-C₁₀ alkenyl (such as substituted with a carboxyl, substituted aryl (3,4-dihydroxyphenyl), or preferably both), preferably wherein when the dashed line in Formula 1e denotes the presence of a bond, one of R₁₃ or R₁₄ is hydrogen,
wherein R₁₅ is substituted C₁-C₁₀ alkyl (such as substituted C₃ alkyl substituted with carboxyl, substituted aryl (3,4-dihydroxyphenyl), or preferably both, such as substituted C₂ alkyl substituted with hydroxyl, carboxyl, or preferably both), substituted C₁-C₁₀ alkoxy (such as substituted C₃ alkoxy substituted with hydroxyl, carboxyl, unsubstituted alkyl (such as methyl), or preferably hydroxyl, carboxyl, unsubstituted alkyl (such as methyl), preferably hydroxyl and carboxyl, such as substituted C₂ alkoxy substituted with hydroxyl, carboxyl, substituted aryl (such as 3,4-dihydroxyphenyl), —C(O)OR₁₆ wherein R₁₆ is unsubstituted alkyl (methyl), or preferably hydroxyl and carboxyl, carboxyl and substituted aryl (such as 3,4-dihydroxyphenyl), —C(O)OR₁₆ and substituted aryl (such as 3,4-dihydroxyphenyl)], substituted aroxy [such as 2,3-dihydroxyphenyl], substituted heterocyclyl [such as 2,3-dihydro-2H-pyran-4-yl substituted with hydroxyl, carboxyl, or preferably both].

In some forms of the methods, for Formula 1e,
the dashed line denotes the presence of a bond,
R₂ and R₃ are hydroxyl,
R₄, R_{A}, and R_{B} are hydrogen,
R₁₃ and R₁₄ are independently hydrogen or —C(O)R₁₅, wherein R₁₅ is substituted C₁-C₁₀ alkoxy (such as substituted C₃ alkoxy substituted with hydroxyl, carboxyl, or both.

In some forms of the methods, the compounds of Formula I are one or more compounds shown below:

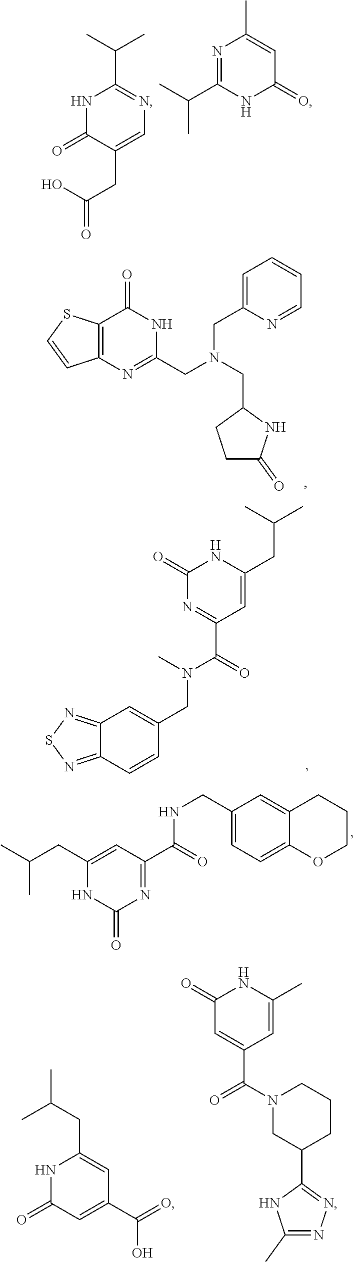

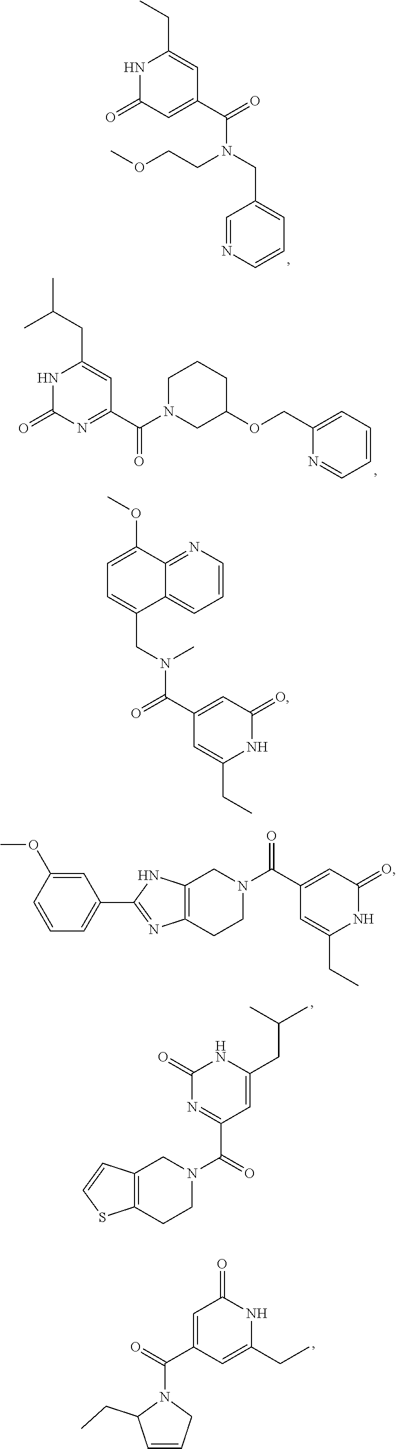

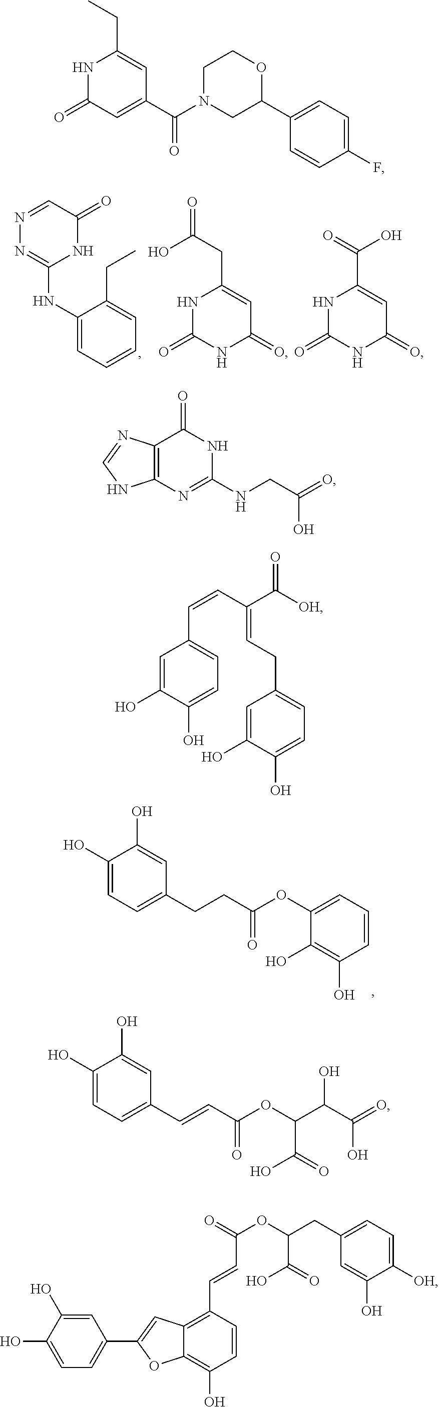

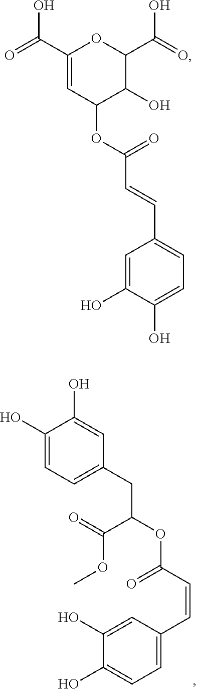 [SMILES: O=C(/C=C/c1ccc(O)c(O)c1)OC1C=C(C(=O)O)OC(C(=O)O)C1O ; COC(=O)C(Cc1ccc(O)c(O)c1)OC(=O)/C=C\c1ccc(O)c(O)c1]

In some forms of the methods, the compounds have a structure shown

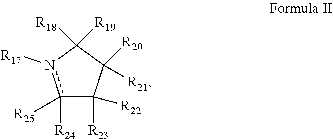

wherein:
the dashed line in Formula II represents the presence or absence of a bond,
R₁₇ is absent, hydrogen, substituted alkyl [such as substituted C₁-C₁₀ alkyl such as arylalkyl (e.g. (3-fluorophenyl)methyl); C₁-C₁₀ alkyl-C(O)—NR₂₆R₂₇, wherein NR₂₆R₂₇ combine to form a substituted heterocyclyl)], or unsubstituted alkyl,
R₁₈ and R₁₉ are independently carboxyl, substituted alkyl [such as substituted C₁-C₁₀ alkyl such as substituted C₁ alkyl substituted with a tertiary amine], hydrogen, or R₁₈ and R₁₉ combine to form an oxo- (═O),
R₂₀ and R₂₁ are independently hydrogen; —NR₂₈C(O)R₂₉, wherein R₂₈ is hydrogen, unsubstituted alkyl, substituted alkyl, R₂₉ is substituted alkyl [such as substituted C₁-C₁₀ substituted with unsubstituted aroxy]; unsubstituted aryl;
   oxo- (═O),
R₂₂ and R₂₃ are independently hydrogen, hydroxyl, or R₂₂ and R₂₃ together with the carbon atom to which they are bonded form a substituted C₃-C₂₀ cyclyl[such as substituted C₃-C₂₀ cyclyl substituted with a hydroxyl],
R₂₄ and R₂₅ are independently absent, hydrogen, substituted alkyl [such as substituted C₁-C₁₀ alkyl such as arylalkyl (e.g. (phenylmethyl)], hydroxyl, unsubstituted alkyl [such as n-butyl], or R₂₄ and R₂₅ combine to form an oxo- (═O),
or R₂₂ and R₂₅ are hydrogen, and R₂₃ and R₂₄ form —O—.

In some forms of the methods, the compounds of Formula II are one or more compounds shown below:

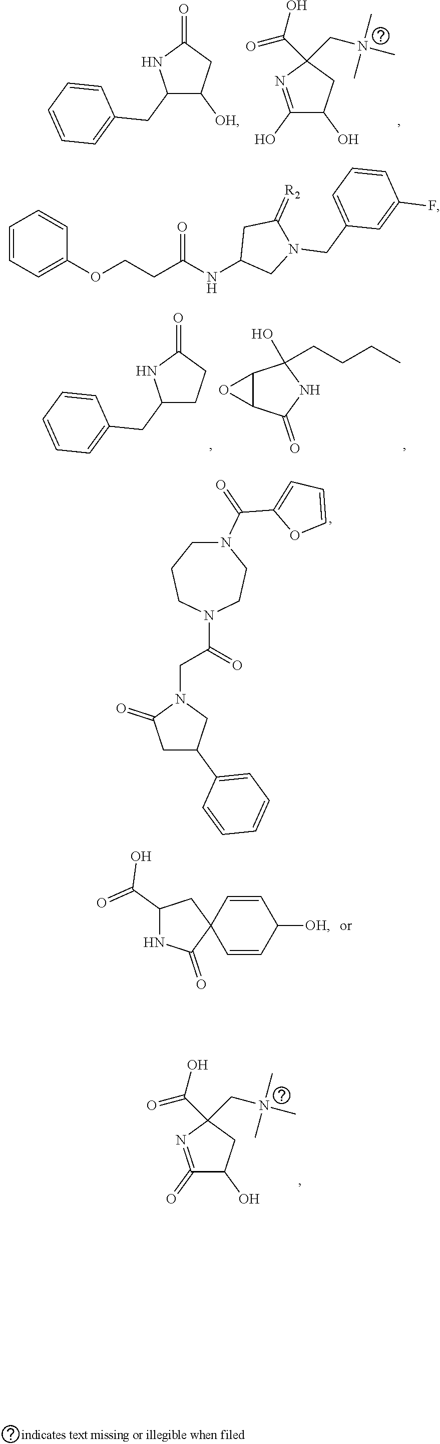

In some forms of the methods, the compounds have a structure shown below:

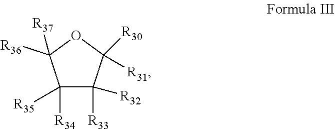

wherein:
R₃₀ and R₃₁ are independently hydrogen, substituted heteroaryl [such as substituted heteroaryl substituted with —NH₂], substituted alkyl [such as substituted C₁-C₁₀ alkyl substituted with hydroxyl, such as carbinol (—CH₂OH), or phosphate], hydroxyl, or phosphate,
R₃₂, R₃₃, R₃₄, and R₃₅ are independently hydrogen or hydroxyl, wherein preferably wherein R₃₃ and R₃₄ are hydroxyl, and
R₃₆ and R₃₇ are independently hydrogen, substituted alkyl [such as substituted C₁-C₁₀ alkyl substituted with sulfonium; unsubstituted C₁-C₁₀ alkoxy such as methoxy; —NHR₃₈, wherein R₃₈ is a substituted C₁-C₅ alkyl substituted with carboxyl and phenyl; or phosphate], or substituted heteroaryl [such as substituted heteroaryl substituted with —NH₂; oxo- (═O), unsubstituted C₁-C₁₀ alkyl (e.g. methyl), or carboxyl].

In some forms of the methods, the compounds of Formula III are one or more compounds shown below:

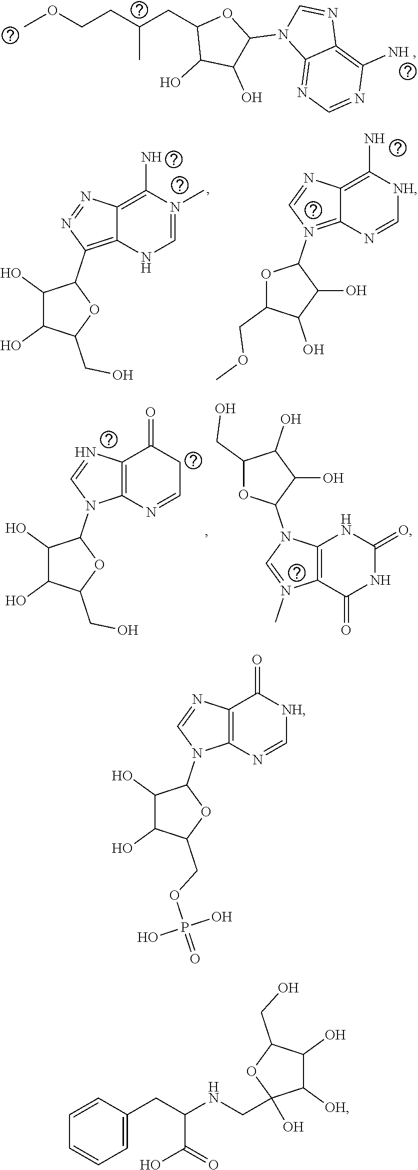

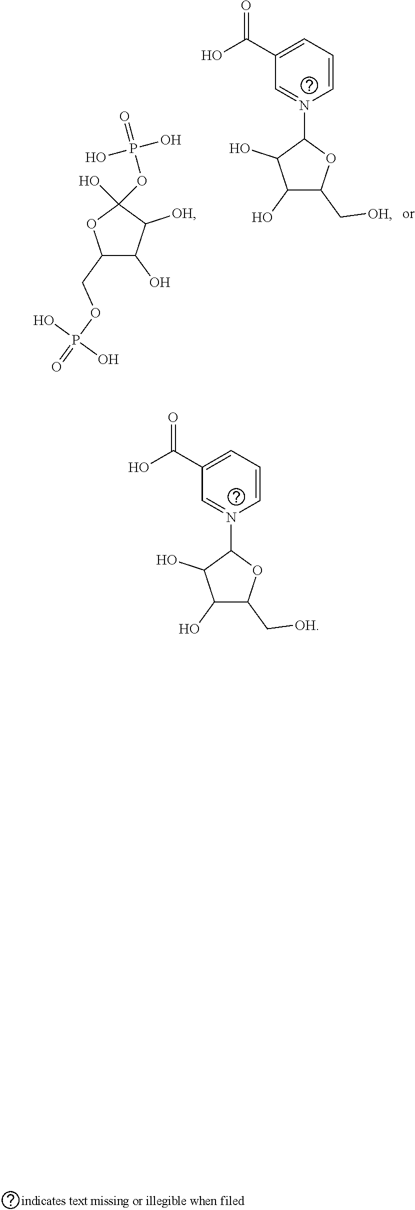

In some forms of the methods, the compounds have a structure shown below:

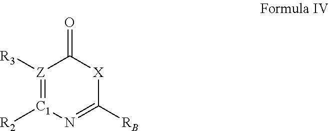

wherein for Formula IV:
X is NH or N—,
C₁ and Z are carbon,
R_{B} is halogen [such as Br], hydrogen, —NR₅R₆, wherein R₅ and R₆ are independently hydrogen or substituted C₁-C₁₀ alkyl [such as substituted C₁ alkyl substituted with a carboxyl], and
C₁R₁ and ZR₃ together form an unsubstituted heteroaryl [such as unsubstituted five-membered heteroaryl], substituted heteroaryl [such as substituted five-membered heteroaryl], or substituted heterocyclyl [such as substituted six-membered heterocyclyl], optionally wherein R₁ and R₃ are nitrogen.

In some forms of the methods, the compounds of Formula IV are one or more compounds shown below:

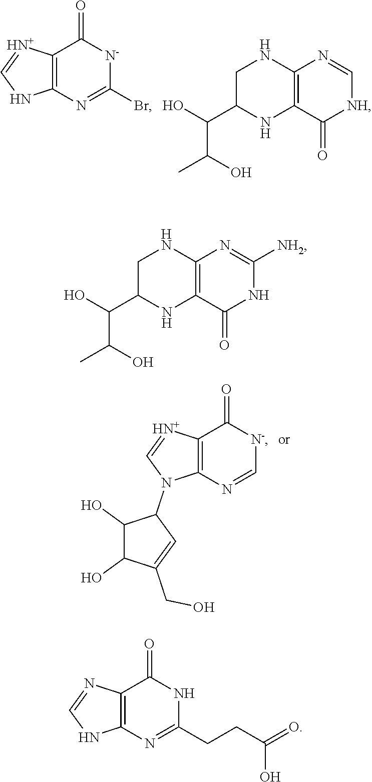

In some forms of the methods, the compounds have a structure shown below:

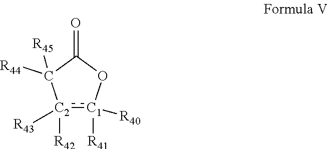

wherein for Formula V:
the dashed line denotes the presence or absence of a bond,
C₁, C₂, and C₃ are carbon atoms,
R₄₀-R₄₅ are independently present or absent depending on the valency of the carbon atom to which they are bonded, and
a) C₁R₄₀ and C₁R₄₁ together form a substituted C₃-C₂₀ cyclyl or unsubstituted C₃-C₂₀ cyclyl, and C₁R₄₁ and C₂R₄₂ together form a substituted C₃-C₂₀ cyclyl or unsubstituted C₃-C₂₀ cyclyl, R₄₃ and R₄₄ are hydrogen, and
R₄₅ is substituted alkyl [such as substituted C₁-C₁₀ alkyl substituted with a tertiary amine],
b) C₁R₄₀ and C₁R₄₁ together form a substituted heterocyclyl[such as substituted heterocyclyl substituted with oxo- (═O) and preferably fused with a substituted aryl] or substituted heterocyclyl, R₄₂ and R₄₃ are hydrogen, and
R₄₄ and R₄₄ are unsubstituted alkyl [such as unsubstituted C₁-C₁₀ alkyl, such as methyl], or
c) R₄₀-R₄₅ are independently hydrogen, substituted aryl, unsubstituted aryl, substituted alkyl, unsubstituted alkyl, hydroxyl, substituted alkenyl, unsubstituted alkenyl, or R₄₄ and R₄₅ together form a double bond that is bonded to C₃.

In some forms of the methods, the compounds of Formula V are one or more compounds shown below:

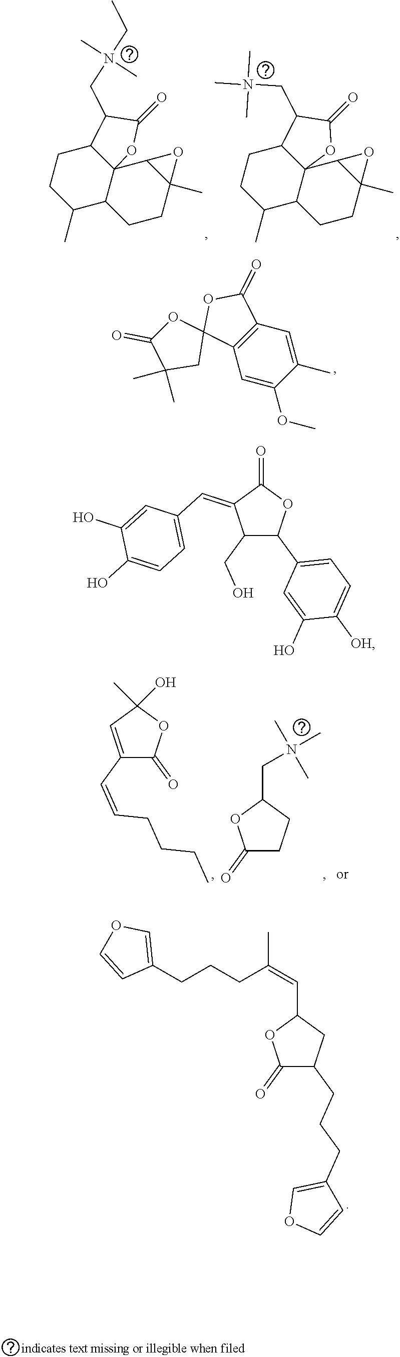

In some forms of the methods, the compounds have a structure shown below:

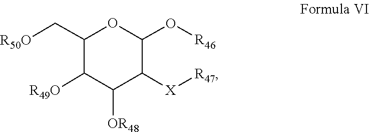

wherein for Formula VI,
X is O or NR₅₁, wherein R₅₁ is hydrogen, unsubstituted alkyl, or substituted alkyl,
R₆ is substituted alkyl[such as substituted C₁-C₁₀ alkyl substituted with substituted C₃-C₂₀ cyclyl, unsubstituted aryl, or substituted C₁ alkyl directly bonded to a carboxyl], substituted aryl, unsubstituted alkyl, or unsubstituted aryl,
R₄₇ is acyl, hydrogen, substituted alkyl, unsubstituted alkyl, substituted aryl, or unsubstituted aryl, and
R₄-R₅₀ are independently hydrogen, substituted alkyl, unsubstituted alkyl, substituted aryl, or unsubstituted aryl.

In some forms of the methods, the compounds of Formula VI are one or more compounds shown below:

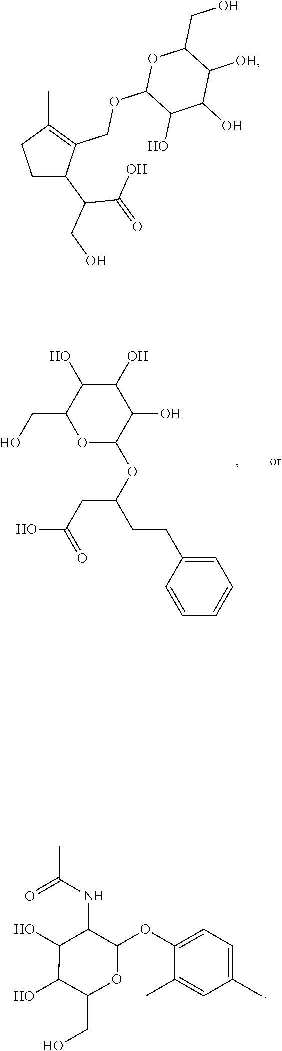 [SMILES: CC1=C(COC2OC(CO)C(O)C(O)C2O)C(C(CO)C(=O)O)CC1 ; O=C(O)CC(CCc1ccccc1)OC1OC(CO)C(O)C(O)C1O ; CC(=O)NC1C(Oc2ccc(C)cc2C)OC(CO)C(O)C1O]

In some forms of the methods, the compounds have a structure shown below:

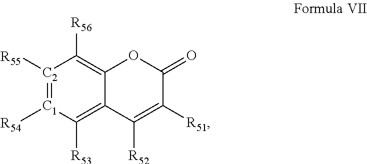

wherein for Formula VII,
C₁ and C₂ are carbon atoms,
R₅₁-R₅₆ are independently hydrogen, substituted alkyl, unsubstituted alkyl, substituted alkoxy, unsubstituted alkoxy, substituted aryl, unsubstituted aryl, substituted heteroaryl, unsubstituted heteroaryl, hydroxyl, halogen, —CN, —NO₂, —SH, etc., or C₁R₅₄ and C₂R₅₅ together form a substituted heteroaryl, unsubstituted heteroaryl, substituted aryl, unsubstituted aryl, substituted C₃-C₂₀ cyclyl, unsubstituted C₃-C₂₀ cyclyl, substituted heterocyclyl, or unsubstituted heterocyclyl.

In some forms of the methods, the compounds of Formula VII are one or more compounds shown below:

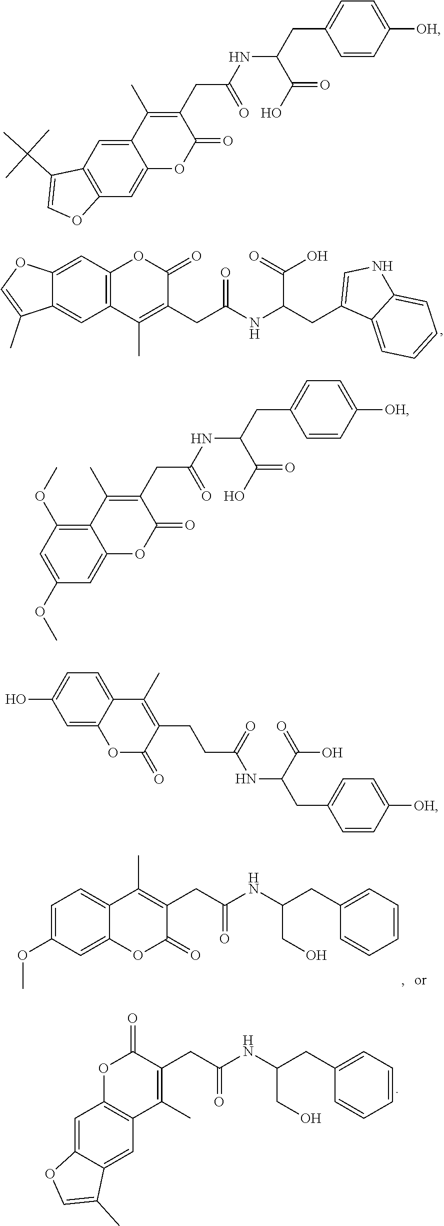

In some forms of the methods, the compounds are one or more compounds shown below:

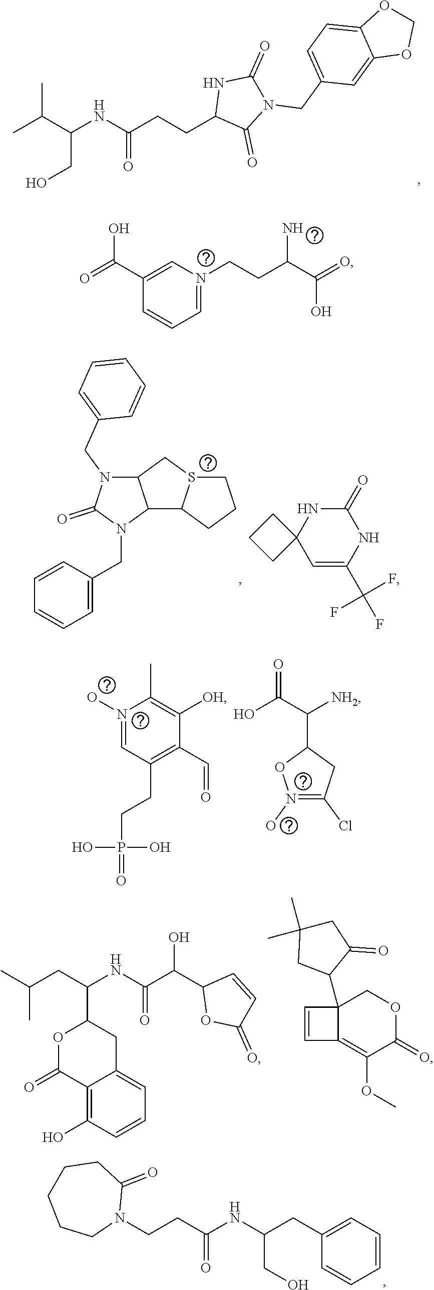

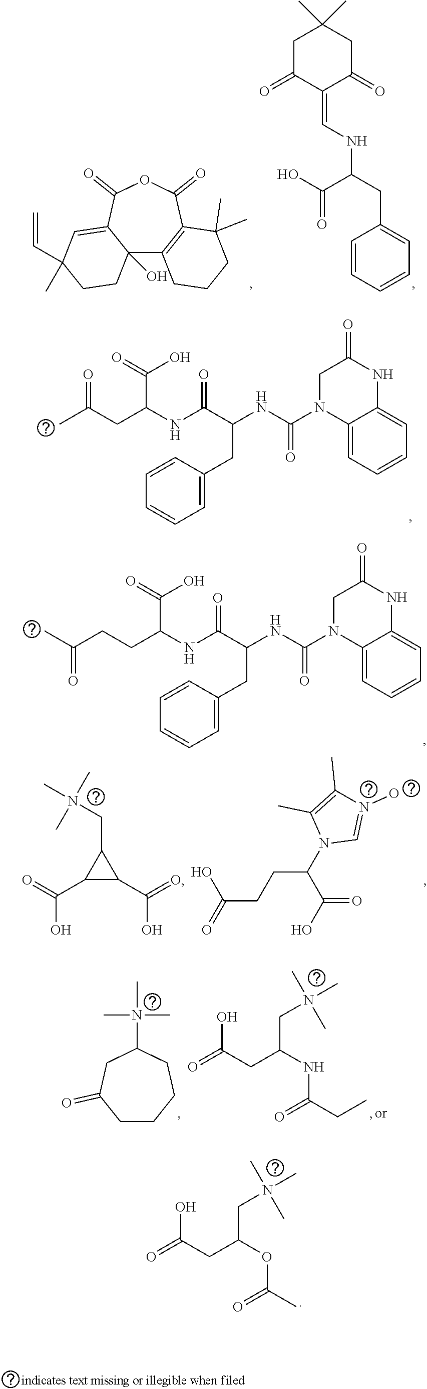

B. Viral infection

The disclosed methods also include treating, preventing, or reducing a viral infection in a patient, involving providing a formulation containing a compound or a pharmaceutically acceptable salt thereof, in an effective amount to treat, prevent, or reduce the viral infection.

In some forms of the methods, the compound has the structure:

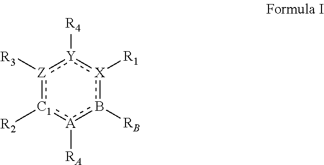

wherein:
C₁ is a carbon atom,
A, B, X, Y, and Z are independently carbon or nitrogen,
A, B, C₁, X, Y, and Z are bonded to none, one, or two hydrogen atoms according to valency,
the dashed lines in Formula I denote the presence or absence of a bond,
R₁, R_{B}, R_{A}, R₂, R₃, and R₄ are independently absent, hydrogen, oxo-(═O), hydroxyl, substituted alkyl [such as substituted C₁-C₁₀ alkyl (such as isobutyl)], unsubstituted alkyl, substituted aryl, unsubstituted aryl, substituted heteroaryl [such as substituted with unsubstituted C₁-C₁₀ alkyl (such as methyl), unsubstituted heterocyclyl (such as pyrrolidin-1-yl)], unsubstituted heteroaryl, substituted C₃-C₂₀ cyclyl, unsubstituted C₃-C₂₀ cyclyl, —NR₅R₆, —C(O)NR₉R₁₀,
R₅ and R₆ are independently hydrogen, unsubstituted aryl, substituted aryl, substituted heteroaryl, unsubstituted heteroaryl,
R₉ and R₁₀ are independently hydrogen, substituted alkyl [such as heterocyclylalkyl], unsubstituted alkyl, or NR₉R₁₀ together form a substituted heterocyclyl, and
at least one of XR₁, BR_{B}, AR_{A}, C₁R₂, ZR₃, and YR₄ is —C(O)— and an adjacent XR₁, BR_{B}, AR_{A}, C₁R₂, ZR₃, and YR₄ is —NH—.

In some forms of the methods, the compound is as described above, except that in Formula I,
R₁, R_{B}, R_{A}, R₂, R₃, and R₄ are independently absent, hydrogen, oxo-(═O), substituted C₁-C₁₀ alkyl (such as isobutyl), substituted heteroaryl (such as substituted with unsubstituted C₁-C₁₀ alkyl (such as methyl), unsubstituted heterocyclyl (such as pyrrolidin-1-yl), or preferably both), —NR₅R₆, —C(O)NR₉R₁₀,
R₅ and R₆ are independently hydrogen or substituted aryl,
R₉ and R₁₀ are independently hydrogen or substituted alkyl (such as heterocyclylalkyl), and
at least one of XR₁, BR_{B}, AR_{A}, C₁R₂, ZR₃, and YR₄ is —C(O)— and an adjacent XR₁, BR_{B}, AR_{A}, C₁R₂, ZR₃, and YR₄ is —NH—.

C. New Chemical Entities

Also described are compounds having the structure:

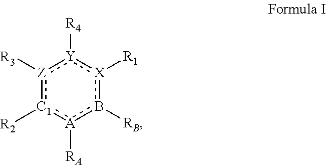

wherein
C₁ is a carbon atom,
A, B, X, Y, and Z are independently carbon or nitrogen,
A, B, C₁, X, Y, and Z are bonded to none, one, or two hydrogen atoms according to valency,
the dashed lines in Formula I denote the presence or absence of a bond, and
a) wherein the moiety

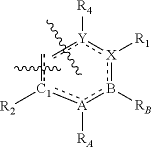

has the structure

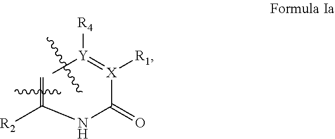

wherein
i) X is carbon, Y is carbon, Z is nitrogen, R₃ is absent, and for Formula Ia,
R₁ is hydrogen,
R₂ is —NR₅R₆, or substituted aryl [such as substituted with unsubstituted C₁-C₁₀ alkoxy (such as methoxy)], wherein R₅ and R₆ are independently hydrogen, unsubstituted aryl, substituted aryl (such as substituted with unsubstituted C₁-C₁₀ alkoxy (such as methoxy), hydroxyl, halogen, unsubstituted anilinyl, unsubstituted aroxy, unsubstituted heterocyclyl (such as 4-morpholinyl), carboxyl, acyl), unsubstituted heteroaryl, substituted heteroaryl (such as substituted with carboxyl, or —NR₇R₈, preferably wherein R₇ and R₈ are both hydrogen), and wherein one of R₅ and R₆ is hydrogen, and
R₄ is substituted aryl (such as substituted with unsubstituted C₁-C₁₀ alkoxy (such as methoxy), unsubstituted C₁-C₁₀ alkyl (such as methyl), or a combination thereof), substituted heteroaryl (such as substituted with unsubstituted C₁-C₁₀ alkyl (such as methyl), unsubstituted C₁-C₁₀ alkoxy (such as methoxy), unsubstituted heterocyclyl (such as 4-morpholinyl, 1-pyrrolidinyl), or a combination thereof, such as unsubstituted C₁-C₁₀ alkyl (such as methyl) and unsubstituted heterocyclyl (such as 4-morpholinyl), or —NR₅R₆, wherein R₅ and R₆ are independently hydrogen, or substituted heteroaryl [such as substituted with carboxyl],
ii) X is nitrogen, R₁ is absent, Y is carbon, Z is carbon, and for Formula Ia,
R₂ is substituted C₁-C₁₀ alkyl (such as isobutyl, 2-methyl-3-hydroxypropyl), —NR₅R₆, wherein R₅ and R₆ are independently hydrogen; substituted heteroaryl (such as pyridin-4-yl substituted with —NH₂); or unsubstituted aryl,
R₄ is —C(O)NR₉R₁₀, substituted heteroaryl (such as substituted with unsubstituted C₁-C₁₀ alkyl such as methyl), unsubstituted heteroaryl, or substituted aryl (such as substituted with unsubstituted C₁-C₁₀ alkoxy (such as methoxy)), and
wherein R₉ and R₁₀ are independently hydrogen, substituted alkyl (such as 2-hydroxyethyl), or NR₉R₁₀ together form a substituted heterocyclyl (such as substituted with hydroxyl),
b) wherein the moiety

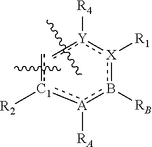

has the structure

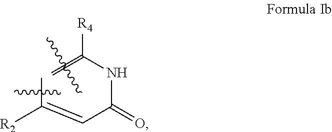

wherein
   Z is nitrogen, R₃ is absent, and for Formula Ib,
   R₂ is —NR₅R₆ wherein R₅ and R₆ are independently hydrogen;
unsubstituted aryl; substituted heteroaryl (such as substituted with —NR₇R₈, preferably wherein R₇ and R₈ are both hydrogen), preferably wherein at least one of R₅ and R₆ is unsubstituted aryl, or substituted heteroaryl,
   R₄ is substituted aryl (such as substituted with unsubstituted C₁-C₁₀ alkoxy (such as methoxy)),
c) wherein the moiety

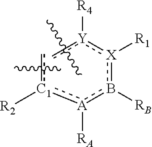

has the structure

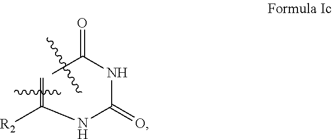

wherein
for Formula I, Z is carbon, R₃ is hydrogen,
R₂ is substituted C₁-C₁₀ alkyl (such as substituted C₁ alkyl directly bonded to —NR₅ₐR₆ₐ, wherein R₅ₐ and R₆ₐ are independently unsubstituted C₁-C₁₀ alkyl (such as methyl), arylalkyl substituted with —NH₂, or both; —NR₅R₆, wherein R₅ and R₆ are independently hydrogen; substituted aryl (such as phenyl substituted with hydroxyl).

In some forms, the compounds have a structure shown below:

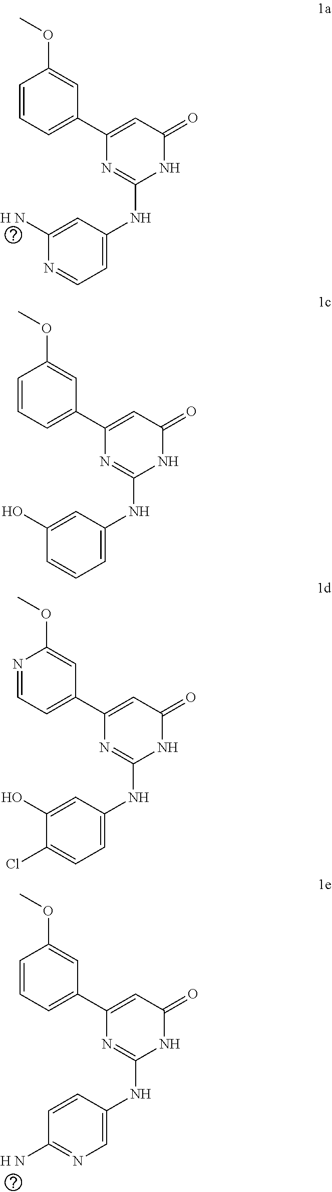

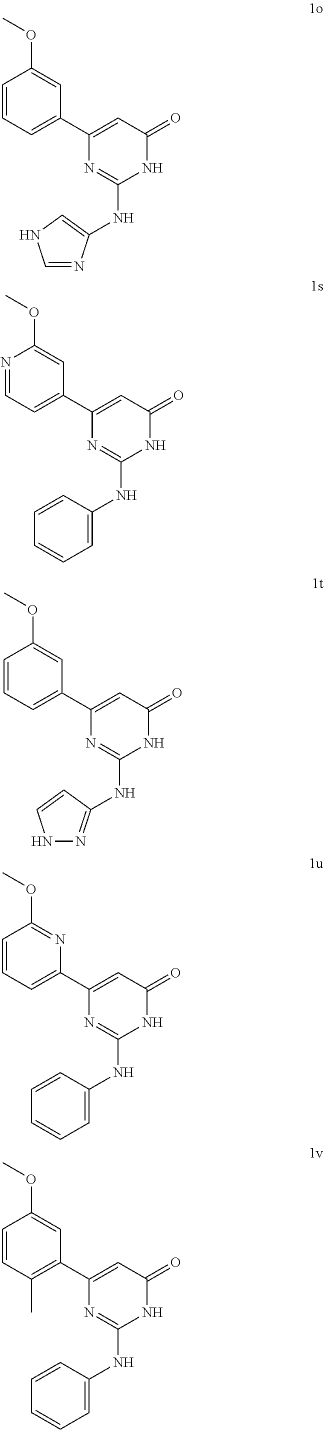 [SMILES: COc1cccc(-c2cc(=O)[nH]c(Nc3c[nH]cn3)n2)c1 ; COc1cc(-c2cc(=O)[nH]c(Nc3ccccc3)n2)ccn1 ; COc1cccc(-c2cc(=O)[nH]c(Nc3cc[nH]n3)n2)c1 ; COc1cccc(-c2cc(=O)[nH]c(Nc3ccccc3)n2)n1 ; COc1ccc(C)c(-c2cc(=O)[nH]c(Nc3ccccc3)n2)c1]

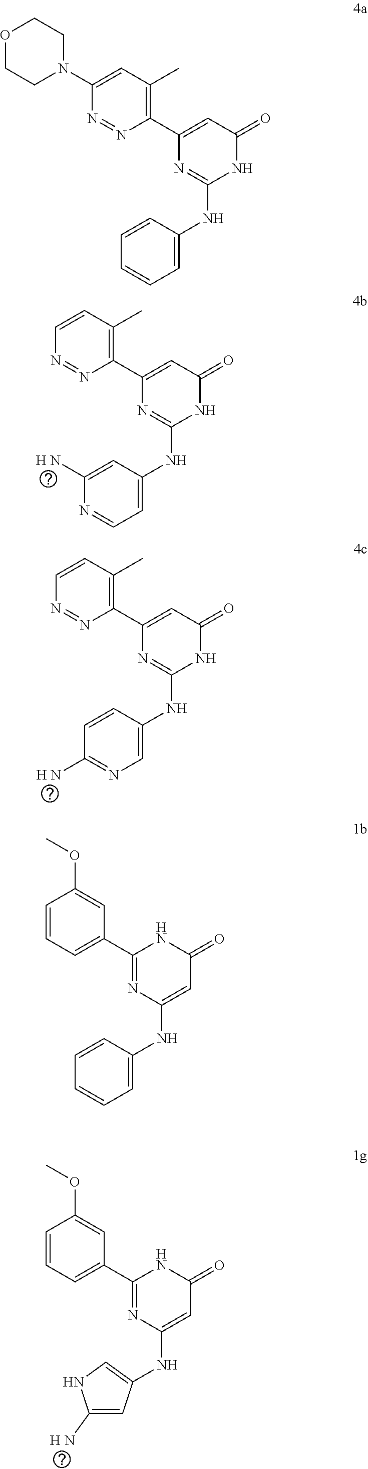

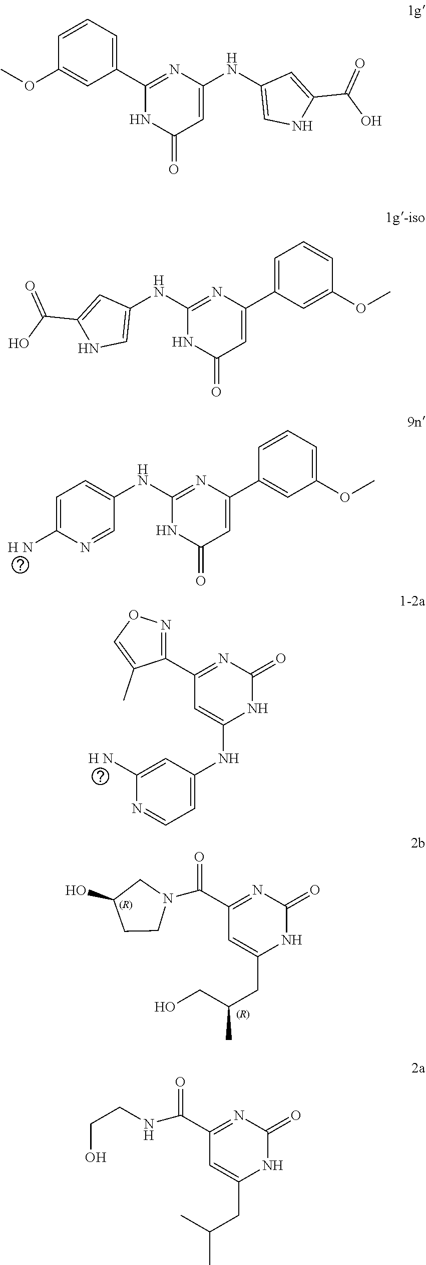

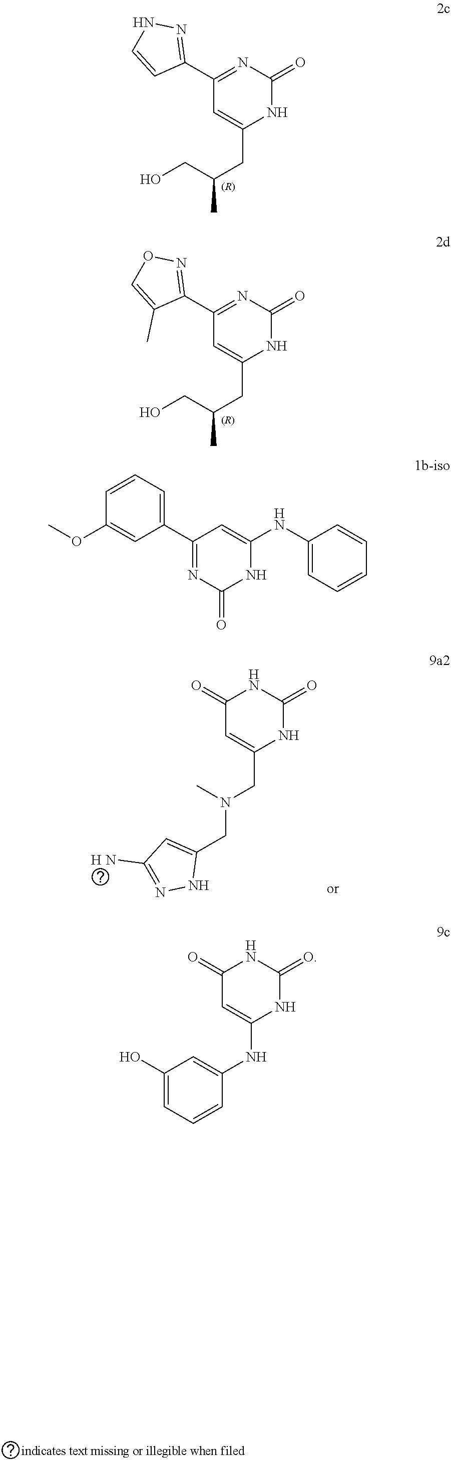

D. Formulations

The compounds described herein can be formulated for enteral, parenteral, topical, or pulmonary administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. See, e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

These formulations can take the form of solutions, suspensions, emulsion, gel, cream, lotion, transdermal patch, oils, tablets, pills, capsules, powders, sustained-release formulations such as nanoparticles, microparticles, etc., and the like.

I. Parenteral Formulations

The compounds described herein can be formulated for parenteral administration. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

If for intravenous administration, the compositions are packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine, and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

If needed, the formulation can be buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

1. Controlled Release Formulations

The parenteral formulations described herein can be formulated for controlled release including immediate release, delayed release, extended release, pulsatile release, and combinations thereof.

(a) Nano- and Microparticles

For parenteral administration, the one or more compounds, and optional one or more additional active agents, can be incorporated into microparticles, nanoparticles, or combinations thereof that provide controlled release of the compounds and/or one or more additional active agents. In forms wherein the formulations contains two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

(b) Method of Making Nano- and Microparticles

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some forms, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some forms, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

2. Injectable/Implantable Formulations

The compounds described herein can be incorporated into injectable/implantable solid or semi-solid implants, such as polymeric implants. In some forms, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

ii. Enteral Formulations

Oral formulations can include standard carriers such as pharmaceutical grades of mannitol, lactose, sodium saccharine, starch, magnesium stearate, cellulose, magnesium carbonate, etc. Such compositions will contain a therapeutically effective amount of the compound and/or antibiotic together with a suitable amount of carrier so as to provide the proper form to the patient based on the mode of administration to be used.

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

1. Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another form, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another form, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

(a) Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred forms, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred forms, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred form, the acrylic polymer is an acrylic resin lacquer such as that which is commercially available from Rohm Pharma under the tradename EUDRAGIT®. In further preferred forms, the acrylic polymer comprises a mixture of two acrylic resin lacquers commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

(b) Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer, enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### III. Methods of Making and Reagents Therefor

The compounds in the methods and compositions described herein can be synthesized using methods known to those of skill in the art of organic chemistry synthesis. In some forms, some of the compounds can be purchased from one or more commercial vendors.

### IV. Methods of Using

As discussed in more detail elsewhere herein, VEGF-A has been shown to be pro-nociceptive. NRPs are also entry points for several viruses into cells. The disclosed compounds, compositions, and formulations are designed to inhibits neuropilin receptor 1 signaling.

The methods typically include administering a subject in need thereof an effective amount of a disclosed compound, composition, or formulation. As used herein, the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

In some forms, methods that reduce neuropilin receptor 1 signaling are provided. For example, a method of reducing neuropilin receptor 1 signaling can include administering a subject in need thereof an effective amount of a disclosed compound, composition, or formulation to reduce neuropilin receptor 1 signaling. In some forms, the formulation is provided in an amount effective to reduce VEGF-A binding to NRP-1 (i.e., VEGF-A/NRP-1 interaction), or a co-receptor formed therefrom such as a dimeric VEGFR2/NRP-1 co-receptor. In some forms, the formulation reduces activation of a VEGF-A pathway.

The formulation can be effective to reduce, or prevent an increase in, phosphorylation of the VEGFR2 cytoplasmic domain at Y1175. For example, in some forms, a disclosed compound is administered in an effective amount to reduce VEGF-A stimulated increased phosphorylation of VEGFR2.

The effective amount of the compound can be ascertained from assays investigating the inhibition of phosphorylation of vascular endothelial growth factor receptor 2 (VEGFR2) compared to a control that does not contain the compound, as determined by an in-cell Western assay that detects inhibition of VEGFR2 activation. In some forms, the in-cell Western assay involves activation of VEGFR2 with a peptide, such as VEGF-A165. In some forms, the phosphorylation of VEGFR2 involves phosphorylation of a tyrosine residue of VEGFR2, preferably an intracellular tyrosine residue. In some forms, the compound has a half-maximal inhibitory concentration (IC50) of inhibiting a NRP-1/VEGF-A interaction of less than 1,000 μM, or less than 100 μM, or less than 10 μM, or less than 1 μM, or less than 0.1 μM, or less than 0.01 μM or less than 0.001 μM; for example, 0.001 μM-1,000 μM, or 0.001 μM-100 μM, or 0.001 μM-10 μM, or 0.01 μM-1,000 μM, or 0.01 μM-100 μM, or 0.01 μM-10 μM, or 0.1 μM-1,000 μM, or 0.1 μM-100 μM, or 0.1 μM-10 μM, or 1 μM-1,000 μM, or 1 μM-100 μM, or 1 μM-10 μM, or any subrange or specific number therebetween.

In some forms, the subject has neuropathic pain and/or a viral infection. Thus, methods of treating, preventing, or reducing neuropathic pain, a viral infection, or a combination thereof are provided.

A. Neuropathic Pain

The methods can involve providing a formulation containing a compound or a pharmaceutically acceptable salt thereof, in an effective amount to treat, prevent, or reduce neuropathic pain in a patient. The amount can be effective to treat, prevent, or reduce one or more symptoms associated with neuropathic pain, such as allodynia, numbness; prickling or tingling; itching; sharp, jabbing, throbbing or burning pain; increased sensitivity to touch; lack of coordination and falling; muscle weakness; paralysis; heat intolerance; excessive sweating or not being able to sweat; bowel problems; bladder problems; digestive problems; changes in blood pressure causing dizziness or lightheadedness; or a combination thereof.

The neuropathic pain can originate from a variety of sources, including, but not limited to, nerve trauma, peripheral nerve damage, central nerve damage, diabetes mellitus, small fiber neuropathy, multiple sclerosis, HIV infection, or a combination thereof.

The effective amount of the compounds in the treatment, prevention, or reduction of neuropathic pain may be examined by using one or more of the published models of pain/nociception or of neuropathy, especially peripheral neuropathy, known in the art. This may be demonstrated, for example using an animal model which assesses the onset and development of tactile allodynia, described herein. The analgesic activity of the compounds or compositions described herein can be evaluated by any method known in the art. Examples include the Tail-flick test (D'Amour, et al., J. Pharmacol. Exp. and Ther. 1941, 72, 74-79); the Rat Tail Immersion Model, the Carrageenan-induced Paw Hyperalgesia Model, the Formalin Behavioral Response Model (Dubuisson, et al., Pain 1977, 4: 161-174), the Von Frey Filament Test (Kim, et al., Pain 1992, 50: 355-363), the Chronic Constriction Injury, the Radiant Heat Model, and the Cold Allodynia Model (Gogas, et al., Analgesia 1997, 3: 111-118), the paw pressure test of mechanical hyperalgesia (Randall and Selitto, Arch Int Pharmacodyn 1997, 111: 409-414; Hargreaves, et al., Pain 1998, 32: 77-88). An in vivo assay for measuring the effect of test compounds on the tactile allodynia response in a rat model of malfunctioning nerves is described in Example 2.

B. Viral Infections

The methods can involve providing a formulation containing a compound or a pharmaceutically acceptable salt thereof, in an effective amount to treat, prevent, or reduce viral infection in a patient. In some forms, the viral infection can be caused by a virus that enters cells through a mechanisms that involves NRP-1. In some forms, the viral infection involves a host's respiratory system. Preferably, the virus is SARS-CoV-2, human T-cell lymphotropic virus type 1, cytomegalovirus, Epstein Barr Virus, and Lujo Virus. In some forms, the virus is SARS-CoV-2.

In some forms, the amount is effective to reduce viral infection of cells of the subject. For example, in some forms, the compound is effective to block viral entry into cells. In some forms, the subject is infected with virus, but does not have any symptoms.

The amount can be effective to treat, prevent, or reduce one or more symptoms associated with a viral infection. Symptoms of, for example, SARS-CoV-2, such as fever, congestion in the nasal sinuses and/or lungs, runny or stuffy nose, cough, sneezing, sore throat, body aches, fatigue, shortness of breath, chest tightness, or wheezing when exhaling, or a combination thereof.

In some forms, determination of an effective amount may include in vitro assays such testing for inhibition of VSV-eGFP-SARS-CoV-2, e.g., in Vero-E6-TMPRSS2 cells, which overexpress the transmembrane serine protease 2.

C. Treatment Regimens

In some forms, the formulations described herein can be administered once a month, more frequently than once a month, once a week, more frequently than once a week, once a day, or several times a day. In some forms, the administering the formulation repeated one month, one week, one day, one hour, two hours, three hours, four hours, five hours, six hours, seven hours, eight hours, nine hours, ten hours, eleven hours, or twelve hours after administration of a prior dose.

### EXAMPLES

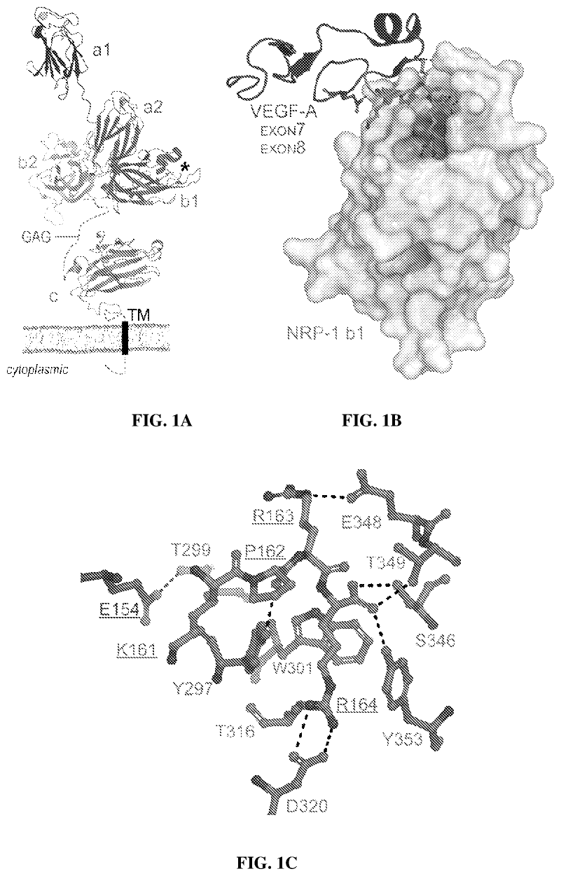

Neuropilins (NRPs) are cell surface receptors for secreted glycoproteins with roles in neural outgrowth, cardiovascular development, immune response, as well as tumor growth and vascularization (Pellet-Many, et al., Biochem J 2008, 411 (2), 211-26; Plein, et al., Microcirculation 2014, 21(4), 315-23). Two neuropilin isoforms, NRP-1 and NRP-2, share ˜44% sequence identity in humans and function in different pathways (Pellet-Many, et al., Biochem J 2008, 411 (2), 211-26; Plein, et al., Microcirculation 2014, 21(4), 315-23). Both share a modular architecture with three extracellular domains, a single transmembrane helix, and a short cytoplasmic tail (FIG. 1A)(Appleton, et al., The EMBO journal 2007, 26 (23), 4902-12).

The a1a2 modules, homologous to CUB (for complement C1r/C1s, Uegf, Bmp1) domains, interact with semaphorin 3A (SEMA3A) to mediate stimulation of growth cone collapse in developing neurons (Pellet-Many, et al., Biochem J 2008, 411 (2), 211-26). The b1b2 modules are homologous to the C-terminal domains of blood coagulation Factors V and VIII (Pellet-Many, et al., Biochem J 2008, 411 (2), 211-26). The c domain, homologous to meprin, A5, and mu-phosphatase (MAM), was initially thought to be involved in dimerization, but is more likely to contribute to complex assembly by positioning the preceding domains away from the membrane (Yelland and Djordjevic, Structure 2016, 24 (11), 2008-2015). The single transmembrane helix contributes to homo- and heterodimerization (Jacob, et al., Oncotarget 2016, 7 (36), 57851-57865) and the C-terminal cytoplasmic tail is thought to contribute to signaling through synectin, stimulating receptor-mediated endocytosis (Plein, et al., Microcirculation 2014, 21 (4), 315-23).

Vascular endothelial growth factor A (VEGF-A) isoforms and other growth factors that terminate in a polybasic stretch ending with an obligatory arginine residue, termed the C-terminal end arginine (CendR) rule (Teesalu, et al., Proc. Natl. Acad. Sci. USA 2009, 106 (38), 16157-62), interact with an acidic pocket formed by loops extending from the beta-barrel of b1 (FIG. 1B) (Lee, et al., Structure 2003, 11(1), 99-108; Parker, et al., The Journal of Biological Chemistry 2012, 287 (14), 11082-9). The structure of the heparin binding domain of the 164 residue isoform of VEGF-A (VEGF-A₁₆₄) confirmed that the C-terminal Arg (Arg164) engages the NRP-1 bi domain pocket with the guanidine forming a bidentate salt-bridge with conserved Asp320 and the carboxylate forming hydrogen bonds to conserved Ser346, Thr349, and Tyr353 (FIG. 1C) (Parker, et al., The Journal of Biological Chemistry 2012, 287(14), 11082-9). It is notable that VEGF-A binds to both NRP-1 and NRP-2, but has higher affinity for NRP-1 due to amino acid substitutions within the first loop region of NRP-1 that provide additional contacts between NRP-1 Thr299 and VEGF-A Glu154 (FIGS. 1B and 1C) (Parker, et al., The Journal of Biological Chemistry 2012, 287 (14), 11082-9). Furthermore, although b1 and b2 are homologous domains, some important residues within the loops of the binding domain are not conserved with the result that the CendR interaction site is not present on b2.

VEGF-A has long been known for its role in blood vessel growth and function, but has more recently been shown to be pro-nociceptive (Llorian-Salvador and Gonzalez-Rodriguez, Frontiers in Pharmacology 2018, 9, 1267). VEGF-A is a selective endothelial cell mitogen that promotes angiogenesis, primarily via interaction with the VEGF receptor VEGFR2, also known as kinase insert domain-containing receptor (KDR) (Djordjevic and Driscoll, Drug Discovery Today 2013, 18 (9-10), 447-55). However, alternative splicing of the VEGF-A gene produces several isoforms of the mature protein containing between 121 and 206 amino acid residues, with VEGF-A₁₆₅ being pro-nociceptive (Hulse, Oncotarget 2017, 8 (7), 10775-10776) via sensitization of transient receptor potential (TRP) channels (Hulse, et al., Neurobiology of Disease 2014, 71, 245-59) and ATP-gated purinergic P2X_{2/3} receptors (Joseph, et al., The Journal of Neuroscience: the official journal of the Society for Neuroscience 2013, 33 (7), 2849-59) on dorsal root ganglion (DRG) neurons. This alternative splicing is dependent on serine-arginine rich protein kinase 1 (SRPK1) which mediates the phosphorylation of serine-arginine rich splice factor (SRSF1) (Hulse, Oncotarget 2017, 8 (7), 10775-10776; Hulse, et al., Neurobiology of Disease 2016, 96, 186-200; Oltean, et al., Biochemical Society Transactions 2012, 40 (4), 831-5). VEGF binding to VEGFR2, a co-receptor for NRP-1, is associated with receptor dimerization and activation that triggers downstream signaling pathways including phosphatidylinositol 3-kinase (PI3-K)/Akt and phospholipase C gamma/extracellular signal-regulated kinase (PLCg/ERK)(Djordjevic and Driscoll, Drug Discovery Today 2013, 18 (9-10), 447-55). Clinical findings that VEGF-A contributes to pain are supported by observations that in osteoarthritis increased VEGF expression in synovial fluids has been associated with higher pain scores (Takano, et al., BMC Musculoskelet Disord 2018, 19 (1), 204). VEGF-A has been reported to enhance pain behaviors in normal, nerve-injured and diabetic animals (Hulse, Oncotarget 2017, 8 (7), 10775-10776; Verheyen, et al., Brain: A Journal of Neurology 2012, 135 (Pt 9), 2629-41).

It is also known that neuropilins are entry points for several viruses, including human T-lymphotropic virus-1 (HTLV-1)(Lambert, et al., Blood 2009, 113 (21), 5176-85) and Epstein-Barr virus (EBV) (Wang, et al., Nat. Commun. 2015, 6, 6240). In both cases, furin processing of viral glycoproteins results in polybasic CendR motifs that directly interact with the VEGF-A site on the NRP-1 b1 domain (Guo and Vander Kooi, The Journal of Biological Chemistry 2015, 290 (49), 29120-6). As of Sep. 21, 2020, COVID-19 has infected more than 31 million people and caused nearly 1 million deaths worldwide (Dong, et al., Lancet Infect Dis 2020, 20 (5), 533-534). This disease is caused by severe acute respiratory syndrome coronavirus 2 (SARS-CoV-2) which primarily gains entry to cells via binding of SARS-CoV-2 Spike glycoprotein to angiotensin converting enzyme 2 (ACE-2) and subsequent endocytosis (Hoffmann, et al., Cell 2020, 181 (2), 271-280 e8; Wang, et al., Cell 2020, 181 (4), 894-904 e9; Yan, et al., Science 2020, 367(6485), 1444-1448). Recent reports have identified additional entry points, including NRP-1 (Cantuti-Castelvetri, et al., bioRxiv 2020, 2020.06.07.137802; Daly, et al., bioRxiv 2020, 2020.06.05.134114). It is through the b1 module that SARS-CoV-2 may gain entry, by taking advantage of the interaction site for VEGF-A. Compared to SARS-CoV-1, the causative agent of SARS, mutation in the furin cleavage site of SARS-CoV-2 results in production of a CendR motif (⁶⁸²RRAR⁶⁸⁵) which was shown to bind to the neuropilin b1 VEGF-A site, suggesting NRP-1 as a possible route of viral entry (Cantuti-Castelvetri, et al., bioRxiv 2020, 2020.06.07.137802; Daly, et al., bioRxiv 2020, 2020.06.05.134114). The importance of NRP-1 is supported by recent evidence of upregulated NRP-1 in lung samples from COVID-19 patients (Cantuti-Castelvetri, et al., bioRxiv 2020, 2020.06.07.137802).

These connections raise the question whether interference with the VEGF-A/NRP-1 signaling pathway by SARS-CoV-2 results in dampened pain. Recently this question has been examined and it has been shown that SARS-CoV-2 Spike protein binding to NRP-1 prevents VEGF-A signaling and reduces neuropathic pain in an animal model (Moutal, et al., bioRxiv 2020). NRP-1 represents a previously unknown target for treating neuropathic pain (Moutal, et al., bioRxiv 2020). Furthermore, targeting NRP-1 also presents a new approach to inhibiting viral entry and/or re-entry into cells to reduce viral load.

Due to its role in cancer, NRP-1 has been a target for drug design for over 20 years. During this time, discovery efforts have focused on the development of NRP-1 antibody therapies (Appleton, et al., The EMBO journal 2007, 26 (23), 4902-12; Liang, et al., J. Mol. Biol. 2007, 366 (3), 815-29; Pan, et al., Cancer Cell 2007, 11(1), 53-67; Weekes, et al., Invest. New Drugs 2014, 32 (4), 653-60; Kim, et al., J. Control. Release 2015, 216, 56-68; Kim, et al., Cancer Lett. 2019, 466, 23-34; Ko, et al., Biomolecules 2020, 10 (6)), including a dual-specificity antibody to VEGFA and NRP-1 (Kim, et al., Cancer Lett. 2019, 466, 23-34; Ko, et al., Biomolecules 2020, (6)), peptides that target transmembrane domain interactions (Jacob, et al., Oncotarget 2016, 7(36), 57851-57865; Roth, et al., Mol. Biol. Cell 2008, 19 (2), 646-54; Nasarre, et al., Oncogene 2010, 29 (16), 2381-92; Arpel, et al., Oncotarget 2016, 7(34), 54723-54732; Albrecht, et al., Front. Oncol. 2020, 10, 519) or the CendR interaction site (Teesalu, et al., Proc. Nati. Acad. Sci. USA 2009, 106 (38), 16157-62; Kim, et al., J. Control. Release 2015, 216, 56-68; Binetruy-Tournaire, et al., The EMBO Journal 2000, 19 (7), 1525-33; Jia, et al., J. Biol. Chem. 2006, 281 (19), 13493-502; von Wronski, et al., J. Biol. Chem. 2006,281 (9), 5702-10; Starzec, et al., Peptides 2007, 28 (12), 2397-402; Vander Kooi, et al., Proc. Natl. Acad. Sci. USA 2007, 104 (15), 6152-7; Getz, et al., ACS Chem. Biol. 2013, 8 (6), 1147-54; Nissen, et al., J. Neurochem. 2013, 127 (3), 394-402; Jia, et al., ChemBioChem 2014, 15 (8), 1161-70; Grabowska, et al., Bioorg. Med. Chem. Lett. 2016, 26 (12), 2843-2846; Richard, et al., Bioorg. Med. Chem. 2016, 24 (21), 5315-5325; Fedorczyk, et al., J. Pept. Sci. 2017, 23 (6), 445-454; Grabowska, et al., Bioorg. Med. Chem. 2017, 25 (2), 597-602; Tymecka, et al., Peptides 2017, 94, 25-32; Tymecka, et al., Eur. J. Med. Chem. 2018, 158, 453-462; Fedorczyk, et al., Molecules 2019, 24 (9); Puszko, et al., MedChemComm 2019, 10 (2), 332-340), as well as small-molecules that target the CendR site (Jarvis, et al., J. Med. Chem. 2010, 53 (5), 2215-26; Novoa, et al., Bioorg. Med. Chem. 2010, 18 (9), 3285-98; Borriello, et al., Cancer Lett. 2014, 349 (2), 120-7; Liu, et al., Bioorg. Med. Chem. Lett. 2014, 24 (17), 4254-9; Starzec, et al., Bioorg. Med. Chem. 2014, 22 (15), 4042-8; Liu, et al., Cancer Lett. 2018, 414, 88-98; Mota, et al., The FEBS Journal 2018, 285 (7), 1290-1304; Powell, et al., J. Med. Chem. 2018, 61(9), 4135-4154). With one exception (Getz, et al., ACS Chem. Biol. 2013, 8 (6), 1147-54), the CendR site targeting peptides contain a CendR motif, even those that are cyclical (Jia, et al., J. Biol. Chem. 2006, 281 (19), 13493-502; Jia, et al., Chem Bio Chem 2014, 15 (8), 1161-70; Grabowska, et al., Bioorg. Med. Chem. Lett. 2016, 26 (12), 2843-2846; Grabowska, et al., Bioorg. Med. Chem. 2017, 25 (2), 597-602) or branched (Tymecka, et al., Eur. J. Med. Chem. 2018, 158, 453-462; Puszko, et al., Med Chem Comm 2019, 10 (2), 332-340). Two of the most well-known small molecule NRP-1 inhibitors, EG00229 and EGO1377, contain a terminal Arg-like moiety and carboxyl group known to be key to interaction with NRP-1 (Jarvis, et al., J. Med. Chem. 2010, 53 (5), 2215-26; Powell, et al., J. Med. Chem. 2018, 61 (9), 4135-4154) as does the newly developed fluorescent compound based on EGO1377 (Conole, et al., Drug Dev. Res. 2020, 81 (4), 491-500). The remaining known small molecule compounds have an arginine-derivative series (Mota, et al., The FEBS Journal 2018, 285 (7), 1290-1304) and several diverse chemotypes such as acylthioureas (Borriello, et al., Cancer Lett. 2014, 349 (2), 120-7), benzamidosulfonamides (Liu, et al., Cancer Lett. 2018, 414, 88-98), bis-guanidines (Novoa, et al., Bioorg. Med. Chem. 2010, 18 (9), 3285-98), or aryl benzylethers (Starzec, et al., Bioorg. Med. Chem. 2014, 22 (15), 4042-8).

To identify compounds that could be used to interrogate the role of these signaling complexes in pain, a virtual screen of nearly 0.5 million compounds against the NRP-1 CendR site was conducted, resulting in nearly 1,000 hits. Here, 35 chemical series of synthetic and natural compounds with lead- or drug-like physico-chemical properties are presented. Pharmacophore models have also been identified based on a subset of the chemical series for guiding design of compounds. The results from a selection of nine tested compounds, show that six of the tested compounds interfere with VEGF-A binding more effectively than EG00229, a known NRP-1 inhibitor, and three interfere with VEGF-A binding as well as the furin-cleaved Spike S1 domain. Furthermore, the nine tested compounds and an additional compound show inhibition of VEGF-A triggered VEGFR2 phosphorylation in a cell-based assay. Finally, two of the tested compounds inhibited antiviral activity.

### Example 1: Virtual Screening to Identify Potential Inhibitors of NRP-1

Materials and Methods

i. Preparation of Receptor Protein and Grid for Virtual

Screening Preparation and virtual screening steps were conducted using Schrödinger Release 2019-3 (Schrödinger, LLC, New York, NY, 2020). The highest resolution structure of the NRP-1 b1 domain was selected for docking (PDB ID: 6fmc) (Powell, et al., J. Med. Chem. 2018, 61 (9), 4135-4154). This structure was prepared using the Protein Preparation Wizard (Sastry, et al., J. Comput-Aided Mol. Des. 2013, 27 (3), 221-34) to remove all water molecules and alternate conformations, add and refine hydrogen atoms, and conduct restrained minimization (OPLS3e force field, convergence to 0.30 Å). There were no residues with alternate conformations within the binding pocket. A 20×20×20 Å grid box was centered on the co-crystallized inhibitor EGO1377 to target the VEGF-A₁₆₅ site. An optional, symmetric constraint was generated that required hit compounds to form a hydrogen bond to the side-chain of Asp 320.

ii. Screening Libraries

The synthetic compound library (DIV) was obtained by combining ChemBridge Diversity Core and Express sets of drug-like compounds. These were prepared for screening in LigPrep using the OPLS3e force field, neutral ionization, desalting, and tautomer generation. If specified, chirality centers were maintained, otherwise up to three chiral variations were generated per atom and ligand. This library contained a total of 210,677 compounds (293,251 conformers). The COlleCtion of Open NatUral producTs (COCONUT) set of open-access natural compounds (Sorokina and Steinbeck, Journal of Cheminformatics 2020, 12 (1)) was downloaded from https://zenodo.org/record/3778405 #.XslD6mhKiUk (on 5/26/20) and prefiltered by excluding compounds with molecular weight ≥500 Da and alogP≥5. LigPrep settings were the same as for the DIV set and the resulting library (NC1) contained 257,166 natural compounds (50,686 conformers). The smaller natural compound library (NC2) library was a curated set of 20,088 natural compounds (23,846 conformers) originally obtained from ZINC15 (Sterling and Irwin, J. Chem. Inf Model. 2015, SS (11), 2324-37). The NC2 library had some overlap with the NC1 library, but nevertheless produced useful results.

iii. Virtual Screening and Scoring

Virtual screens were run for each library against the VEGF-A₁₆₅ binding site of NRP-1 using the Glide virtual screening workflow (Schrödinger, LLC, New York, NY, 2020)(Friesner, et al., J. Med. Chem. 2004, 47(7), 1739-49). For the DIV and NC1 libraries, the default docking settings were accepted, with 10% of compounds at each stage (high-throughput virtual screen, standard precision docking, extra precision docking) resulting in 293 hits for the DIV library and 550 hits for the NC1 library. Because the NC2 library was smaller, it was set to retain 25%, 20%, 15% of the hits at each stage, resulting in 152 hits. The virtual screens were first run without and then with the use of the Asp 320 constraint, but only the constrained hits from NC2 were retained due to strained conformations and lower docking scores for the DIV and NC1 screens. Thus, a total number of 1,147 virtual hit compounds were obtained from four screens.

iv. Docking of Known NRP-1 Targeting Compounds

Representatives of known compound series (Jarvis, et al., J. Med. Chem. 2010, 53 (5), 2215-26; Novoa, et al., Bioorg. Med. Chem. 2010, 18 (9), 3285-98; Borriello, et al., Cancer Lett. 2014, 349 (2), 120-7; Starzec, et al., Bioorg. Med. Chem. 2014, 22 (15), 4042-8; Liu, et al., Cancer Lett. 2018, 414, 88-98; Powell, et al., J. Med. Chem. 2018, 61(9), 4135-4154) were prepared for screening in LigPrep using the OPLS3e force field, neutral ionization, desalting, and tautomer generation. Docking was run against the VEGF-A₁₆s binding site of NRP-1 using Glide XP (Schrödinger, LLC, New York, NY, 2020)(Friesner, et al., J. Med. Chem. 2004, 47 (7), 1739-49).

v. Compound Property Calculations

The following physico-chemical properties were calculated using RDKit (RDKit: Open-source cheminformatics, 2018.09.03): molecular weight in Daltons (Mw, Da), partition coefficient (clogP), number of hydrogen-bond donors and acceptors (HB-D, HB-A), total number of N—H and OH groups (NHOH), number of rotatable bonds (RotB), and total polar surface area (TPSA, Å²). To estimate compound solubility, calculated logS (M) at pH=7.4 was obtained using ChemAxon Aqueous solubility module (ChemAxon).

Results

Virtual screens were conducted against the VEGF-A binding site on the NRP-1 bi domain using three libraries: a ˜211 K synthetic compound library (DIV) from ChemBridge; a ˜257 K natural compound library (NC1) obtained from the COlleCtion of Open NatUral producTs (COCONUT) resource (Sorokina and Steinbeck, Journal of Cheminformatics 2020, 12 (1)); and a ˜20 K (NC2) natural compound library from the ZINC15 database (Sterling and Irwin, J. Chem. Inf Model. 2015, SS (11), 2324-37). The screens were run once without ligand-receptor interaction constraints and repeated with the constraint that compounds form a hydrogen bond to Asp 320, a key residue for coordinating the terminal arginine in the CendR motif (Parker, et al., The Journal of Biological Chemistry 2012, 287 (14), 11082-9). This constraint was used as an attempt to select for compounds that interact in a similar way as observed for VEGF-A (Parker, et al., The Journal of Biological Chemistry 2012, 287 (14), 11082-9), known inhibitors (Vander Kooi, et al., Proc. Natl. Acad. Sci. USA 2007, 104 (15), 6152-7; Jarvis, et al., J. Med. Chem. 2010, 53 (5), 2215-26; Mota, et al., The FEBS Journal 2018, 285 (7), 1290-1304; Powell, et al., J. Med. Chem. 2018, 61 (9), 4135-4154) and modeled SARS-CoV-219 CendR terminal arginine (Daly, et al., bioRxiv 2020, 2020.06.05.134114). However, application of this constraint led to reduced overall scores and strained conformations for most compounds in the DIV and NC1 libraries. Therefore, the analysis are based on the unconstrained screen results for these libraries and both constrained and unconstrained results for the NC2 screen.

i. Selection of Top Compounds

The combined output of the four screens produced a total of 1,147 hits. Compounds from each screen were sorted by Glide XP GScore (kcal/mol) and visually inspected for substructure match of core scaffolds and patterns of chemically reactive moieties while considering the diversity of chemotypes. Compound representatives with scaffold decoration that suggested initial structure activity relationships (SAR) were extracted and grouped into series resulting in a set of nine diverse chemotypes (series). These series include both small synthetic molecules and natural products (Table 1).

TABLE
Hit and reference compounds with annotations of series assignment, binding Mode, origin, and calculated or experimental data.

| | | | | | | | | | | | | | | | | Rel % |
|---|---|---|---|---|---|---|---|---|---|---|---|---|---|---|---|---|
| | Glide | | Source | | Bnd | BBB | log(S) | | | | | Lip- | | | | inhibi- |
| Cmpd | Gscore | Set | Idx | Series | Mode | Score | (7.4) | Mw | cLogP | HBD | HBA | inski | NHOH | RotB | TPSA | tion |
| 1 | −12.1 | DIV | 29267 | 1 | I | 4.2 | −5.2 | 293.33 | 3.2 | 2 | 4 | Y | 2 | 4 | 67 | 56 ± 7 |
| 2 | −11.5 | DIV | 2147 | 2 | I | 4.1 | −3.3 | 279.34 | 0.7 | 2 | 4 | Y | 2 | 5 | 84 | 59 ± 11 |
| 3 | −10.8 | DIV | 37503 | 2 | I | 3.9 | −4.2 | 342.4 | 0.7 | 1 | 6 | Y | 1 | 4 | 95 | nd |
| 4 | −10.4 | DIV | 41088 | 1 | I | 2.7 | −5.0 | 364.41 | 2.1 | 2 | 7 | Y | 2 | 4 | 96 | 68 ± 5 |
| 5 | −10.3 | DIV | 45423 | 1 | I | 2.7 | −5.2 | 378.44 | 2.9 | 2 | 7 | Y | 2 | 5 | 96 | 42 ± 10 |
| 6 | −10.3 | DIV | 42130 | 2 | I | 4.3 | −2.5 | 329.4 | 1.5 | 1 | 5 | Y | 1 | 6 | 92 | nd |
| 7 | −9.8 | DIV | 8757 | 3 | I | 3.2 | −1.7 | 218.22 | 0.9 | 2 | 5 | Y | 2 | 3 | 80 | 18 ± 19 |
| 8 | −9.7 | DIV | 8892 | 3 | I | 3.0 | −1.5 | 204.19 | 0.6 | 3 | 5 | Y | 3 | 2 | 91 | 24 ± 16 |
| 9 | −9.2 | DIV | 9167 | 4 | II | 3.4 | −2.3 | 250.26 | 0.0 | 2 | 5 | Y | 2 | 4 | 95 | 43 ± 8 |
| 10 | −11.3 | NC1 | 15860 | 6 | I | 1.9 | 0.0 | 209.17 | −0.9 | 4 | 5 | Y | 4 | 3 | 124 | nd |
| 11 | −11.0 | NC1 | 34346 | 1 | I | 3.4 | 0.6 | 231.21 | 0.5 | 2 | 4 | Y | 2 | 3 | 96 | 43 ± 16 |
| 12 | −10.9 | NC1 | 21399 | 5 | I | 3.7 | −2.3 | 347.37 | −0.9 | 2 | 5 | Y | 2 | 4 | 99 | nd |
| 13 | −10.7 | NC1 | 36469 | 9 | I | na | na | 358.35 | 2.4 | 5 | 6 | Y | 5 | 7 | 135 | nd |
| 14 | −9.8 | NC2 | 13787 | 7 | II | na | na | 312.27 | −0.1 | 5 | 7 | Y | 5 | 6 | 145 | nd |
| 15 | −9.6 | NC2 | 7454 | 5 | I | 4.6 | 0.6 | 222.29 | 0.4 | 2 | 3 | Y | 2 | 2 | 56 | nd |
| 16 | −9.6 | NC2 | 16482 | 9 | II | na | na | 453.46 | 0.2 | 5 | 5 | Y | 6 | 8 | 171 | nd |
| 17 | −9.4 | NC2 | 13777 | 7 | II^{c} | na | na | 312.23 | −0.4 | 5 | 7 | Y | 5 | 6 | 162 | 12 ± 16 |
| 18 | −9.1 | NC2 | 13788 | 7 | II^{c} | na | na | 312.27 | −0.1 | 5 | 7 | Y | 5 | 6 | 145 | nd |
| 19 | −8.6 | NC2 | 15222 | 8 | II^{c} | na | na | 396.44 | 0.6 | 4 | 5 | Y | 6 | 13 | 176 | nd |
| 20 | −8.4 | NC2 | 15223 | 8 | II | na | na | 396.44 | 0.6 | 4 | 5 | Y | 6 | 13 | 176 | nd |
| SARS-COV2 S1* | | | | | | | | | | | | | | | | 54 ± 11 |
| A | −6.4 | Ref54 | EG00229 | 10 | II^{c} | na | na | 497.58 | 0.8 | 5 | 9 | Y | 7 | 10 | 203 | 33 ± 10 |
| B | −5.8 | Ref61 | EG01377 | 10 | II | na | na | 586.7 | 1.8 | 6 | 8 | N | 9 | 12 | 212 | nd |
| C | −5.2 | Ref56 | 1 | 11 | II^{c} | 3.2 | −7.9 | 444.52 | 4.4 | 3 | 5 | Y | 3 | 3 | 88 | nd |
| D | −4.3 | Ref55 | 32 | 12 | II | na | na | 500.6 | 0.0 | 6 | 6 | Y | 9 | 10 | 190 | nd |
| E | −4.1 | Ref58 | CB- | 13 | na | 5.0 | −3.5 | 390.69 | 4.3 | 2 | 4 | Y | 2 | 8 | 51 | nd |
| | | | 7739526 | | | | | | | | | | | | | |
| F | −3.5 | Ref29 | NRPa-308 | 14 | II | 4.1 | −6.2 | 424.52 | 4.8 | 2 | 4 | Y | 2 | 7 | 85 | nd |

Ref54: Jarvis, et al., J. Med. Chem. 2010, 53 (5), 2215-26; Ref61: Powell, et al., J. Med. Chem. 2018, 61 (9), 4135-4154; Ref56: Borriello, et al., Cancer Lett. 2014, 349 (2), 120-7; Ref55: Novoa, et al., Bioorg. Med. Chem. 2010, 18 (9), 3285-98; Ref58: Starzec, et al., Bioorg. Med. Chem. 2014, 22 (15), 4042-8; Ref59: Liu, et al., Cancer Lett. 2018, 414, 88-98. Compound ranking (Glide Gscore (kcal/mol)) and calculated properties of the top twenty hits with added reference compounds (A-F). Set: screening library; Source_Idx: reference to internal ID in the screening set; Series: chemotype assignment to hit series 1-9 and known compound series 10-14; Bnd Mode: binding Mode I or II (^{c}denotes constrained docking); BBB score: score for compound probability of having CNS exposure; LogS(7.4) predicted solubility (M) at pH 7.4; Mw molecular weight (Da); cLogP predicted lipophilicity coefficient in octanol/water; HBD number of hydrogen-bond donors; HBA number of hydrogen bond acceptors; Lipinski - a binary (Y/N) assignment of complying with Lipinski rule-of-5; NHOH number of polar NH and OH hydrogens; RotB number of rotatable bonds; TPSA total polar surface area (Å2); na denotes not applicable; n.d. indicates value not determined. All properties were calculated using RDKit. Relative % inhibition (ELISA VEGF-NRP-1 assay - see Methods section) with standard error of the mean (n = 11).

While calculated property ranges vary for the chemotype and drug indication, compounds in the series were retained based on the following criteria: molecular weight (Mw)<400 Da, calculated octanol-water partition coefficient (clogP)<3.5, and calculated solubility (clogS) at pH 7.4>−5 M. Consequently, the initial absorption, distribution, metabolism, and excretion (ADME) profile of the series are expected to be acceptable (e.g., good hepatocyte clearance and bioavailability). To assess the probability that hit series compounds are orally available, components and overall compliance with Lipinski rule of 5 (Ro5)(Lipinski, Drug Discov. Today Technol. 2004, 1 (4), 337-41; Shultz, J. Med. Chem. 2019, 62 (4), 1701-1714) were determined. Since the Ro5 guidelines were derived for orally available small molecules, this binary parameter was not utilized for the classification of natural products. Also, since solubility and CNS penetration rules originated from small molecule sets, these calculations are not applicable to natural products and were also omitted. Furthermore, detailed inspection of data of Table 1 revealed that natural product compounds 11, 12, and 15 are in fact small molecule chemotypes. Correspondingly, all molecular descriptors were calculated for these compounds and they were considered part of the small molecule set.

Since one objective of inhibiting the VEGF-A/NRP-1 interaction is disruption of pain signals, the full therapeutic effect can involve drug exposure in the central nervous system (CNS). Correspondingly, the design strategy for these compounds can involve modifications beneficial for crossing the blood-brain barrier (BBB), for example, decreasing the number of hydrogen bond donors and polarity. To supplement traditional medicinal chemistry approaches to increasing CNS exposure, the BBB score algorithm of Weaver, et al., (Gupta, et al., J. Med. Chem. 2019, 62 (21), 9824-9836) was implement as one of the design parameters. Values of the BBB score in the range of 4 to 6 have been shown to correctly predict 90% of CNS drugs (Gupta, et al., J. Med. Chem. 2019, 62 (21), 9824-9836). While the brain/plasma ratio will be experimentally assessed for series representatives, modification of the compounds will be additionally guided by surrogate estimates of passive diffusion (PAMPA) and assessment of efflux and transporter proteins using assays in MDCK cell lines. Overall, the predicted physico-chemical properties of all small molecule hit series (series 1-6) fall within ranges of lead-like and/or drug-like molecules (Table 1).

ii. Series Analysis of Docked Binding Modes

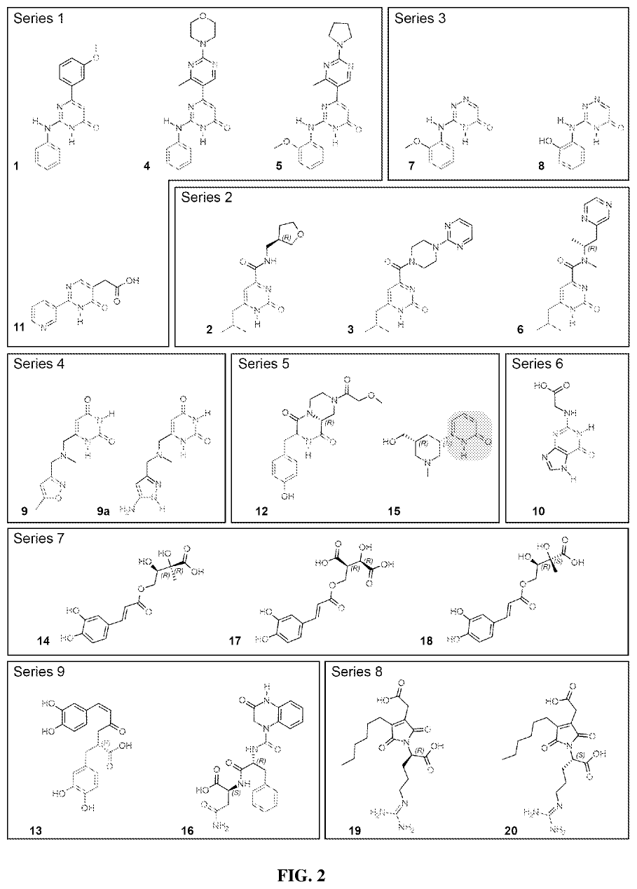

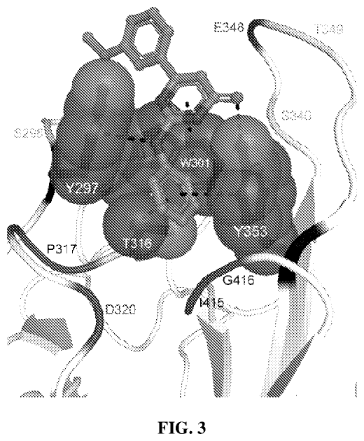

After initial ranking and selection of the top 20 hits, a more detailed analysis of structural and chemical features of the compounds was performed. Chemical structures of all twenty hits and one virtual SAR analog are shown in FIG. 2. Hits are grouped by common core motifs and molecular fragments that engage in productive hydrogen bond (HB) and alkyl/aryl n-contacts within the binding pocket. Inspection of aligned 2-D structures reveals that the 2(1H)-pyridone core of structure 15 (highlighted in FIG. 2) is the minimal motif of all small molecule hits (discussed further below) that can include additional heteroatoms in the ring. This core binds near the top of a central hydrophobic box formed by residues Tyr297, Tyr253, Trp301, and the methyl group of Thr316 (FIG. 3). The analysis revealed that all of the hits bind within this box with the aryl or alkyl (e.g., isobutyl) groups engaged in hydrophobic interactions with these residues. Indeed, it is these hydrophobic interactions that are drivers of the overall binding affinity as judged by the Glide XP Gscore.

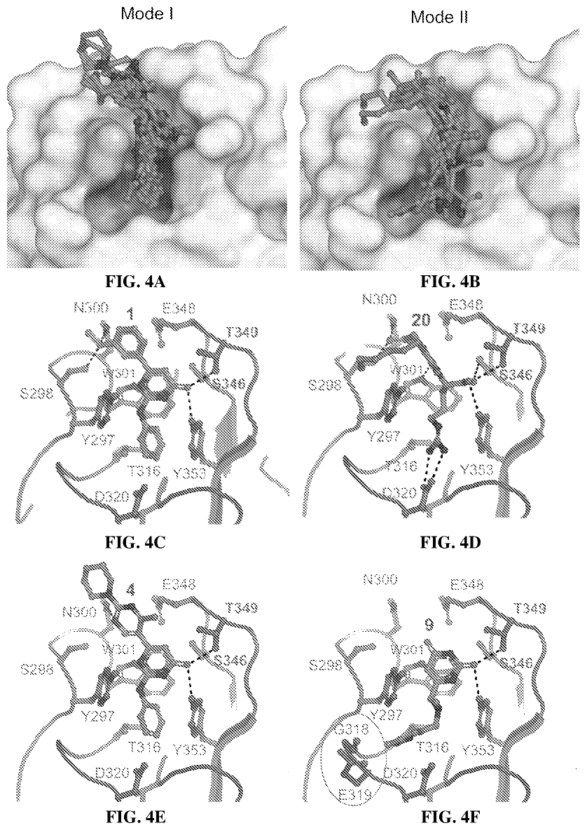

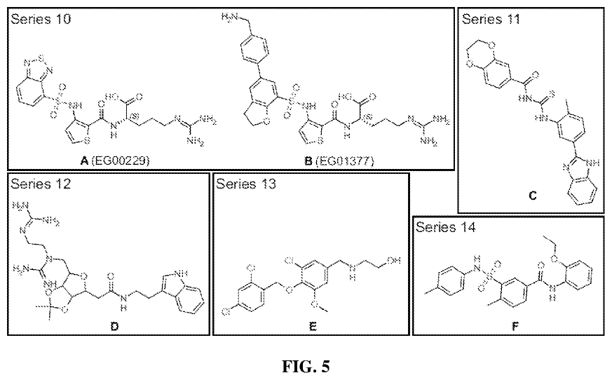

From this central position, the hits extend out of the aryl box in two general binding modes, referred to as Mode I (FIG. 4A) and Mode II (FIG. 4B). Interestingly, it is the core lactam carbonyl group in compounds 1-12, 15, 19-20 that makes potential hydrogen bonds with the hydroxyl groups of Thr349 and Tyr353 (FIGS. 4C and 4D), whereas in 14, 16-18 the carbonyl in the carboxylic group make these contacts, and in 13, 19, 20 the carboxyl makes contacts with S346, T349, and Y353 (FIG. 4E). Thus, all of compounds are able to partially mimic the terminal carboxyl contacts that are considered important for anchoring the terminal Arg of CendR peptides (Teesalu, et al., Proc. Natl. Acad. Sci. USA 2009, 106 (38), 16157-62; Kim, et al., J. Control. Release 2015, 216, 56-68; Binetruy-Tournaire, et al., The EMBO Journal 2000, 19 (7), 1525-33; Jia, et al., J. Biol. Chem. 2006, 281 (19), 13493-502; von Wronski, et al., J. Biol. Chem. 2006, 281 (9), 5702-10; Starzec, et al., Peptides 2007, 28 (12), 2397-402; Vander Kooi, et al., Proc. Natl. Acad. Sci. USA 2007, 104 (15), 6152-7; Nissen, et al., J. Neurochem. 2013, 127 (3), 394-402; Jia, et al., Chem Bio Chem 2014, 15 (8), 1161-70; Grabowska, et al., Bioorg. Med. Chem. Lett. 2016, 26 (12), 2843-2846; Richard, et al., Bioorg. Med. Chem. 2016, 24 (21), 5315-5325; Fedorczyk, et al., J. Pept. Sci. 2017, 23 (6), 445-454; Grabowska, et al., Bioorg. Med. Chem. 2017, 25 (2), 597-602; Tymecka, et al., Peptides 2017, 94, 25-32; Tymecka, et al., Eur. J. Med. Chem. 2018, 158, 453-462; Fedorczyk, et al., Molecules 2019, 24 (9); Puszko, et al., Med Chem Comm 2019, 10 (2), 332-340) (FIG. 1C) or known small molecule mimetics and inhibitors that contain a terminal guanidyl from Arg moiety and carboxylic group (Jarvis, et al., J. Med. Chem. 2010, 53 (5), 2215-26; Powell, et al., J. Med. Chem. 2018, 61 (9), 4135-4154; Conole, et al., Drug Dev. Res. 2020, 81 (4), 491-500).

All of the hit molecules occupying Mode I are synthetic compound chemotypes, including 11, 12, and 15 from the NC1 and NC2 libraries, as noted above. Functional groups in 1, 2, 4, 5, 10, 12, 13 are involved in one or more potential hydrogen bond contacts with polar side chains Ser298 and Asn300 (FIGS. 4C and 4D). No other heteroatoms nor N—H hydrogen in pyrimidone (1, 4, 5, 10, 11), 2-pyrimidone (2, 3, 6), or 4H-1,2,4-triazin-5-one (7, 8) scaffolds are seemingly engaged in productive binding. Such “silent” polar sites provide the opportunity for replacement and enhancement of ADME properties (e.g., oral absorption, systemic/CNS distribution) of these compounds. Interestingly, upon detailed analysis of hydrogen-bond patterns, it was revealed that series 1, 3 but also 2-6 feature H-donor/H-acceptor topology of kinase inhibitors, including the presence of hydrophobic residues found in hinge-binding ATP mimics. Such features warrant the inclusion of kinase selectivity panels in the lead enhancement stage. While kinase activity might be a feature to exclude, the validation experiments support the binding of compounds from the series described herein to the CendR site on NRP-1 (discussed below).

Mode II, with the exception of compound 9, features skeletons of natural compounds. The functional groups in molecules 14 and 16-20 possess extensions toward the base of the pocket that form ionic or hydrogen bond contacts to residue Asp320 (FIG. 4E). Furthermore, compound 9 extends toward the open region of the binding pocket bordered by Gly318 and Glu319 (FIG. 4F). SAR studies for compound 9 show that augmenting these interactions by the replacement of 5-methylisoxazole with 5-aminopyrazole (9a, FIG. 2) leads to an improvement in the Glide XP Gscore of 1.4 kcal/mol. Exploration of the interaction patterns observed in both binding modes can improve binding affinity and compound selectivity.

It is noted that molecules 13 and 16, while having the key pharmacophores present, had their geometry altered during the ligand preparation, likely a result of missing or incorrect chiral information in the COCONUT library, a known potential issue (Sorokina and Steinbeck, Journal of Cheminformatics 2020, 12 (1)). The hydroxycinnamyl group in 13 is present in the less stable Z-conformation and the chiral center at phenylalanine in 16 has inverted to (R)-configuration. Such alterations made both analogs unavailable from commercial sources of natural products. However, due to the availability of both precursors, derivatives 13 and 16 can be synthesized. Finally, the last two compounds (19, 20) are ionic, moderately reactive compounds which are not considered to be drug-like. Nevertheless, since both match the key features of CendR peptides (N-acylarginine), they provide valuable points for SAR.

iii. Comparison to Known Small Molecules

To provide a basis for comparison with small molecules reported by others, six compounds that also target the NRP-1 CendR site (Jarvis, et al., J. Med. Chem. 2010, 53 (5), 2215-26; Novoa, et al., Bioorg. Med. Chem. 2010, 18 (9), 3285-98; Borriello, et al., Cancer Lett. 2014, 349 (2), 120-7; Starzec, et al., Bioorg. Med. Chem. 2014, 22 (15), 4042-8; Liu, et al., Cancer Lett. 2018, 414, 88-98; Mota, et al., The FEBS Journal 2018, 285 (7), 1290-1304; Powell, et al., J. Med. Chem. 2018, 61 (9), 4135-4154) were docked, and their physico-chemical properties were also calculated. Based on chemotypes, these six molecules were assigned into additional series, namely series 10-14 (FIG. 5). These molecules all exhibited less favorable docking scores compared to the hits identified herein (Table 1). Moreover, several of compounds A-F feature functional groups known for contributing to suboptimal physico-chemical and ADME properties, such as solubility, low intestinal absorption, metal chelation, and lability in liver microsomes or hepatocytes (Table 1). Docking poses for compounds A-F except E adopted Mode II binding.

### Example 2: Validation of Inhibitors of NRP-1 Identified from the Virtual Screening

Materials and Methods

i. ELISA-Based NRP1-VEGF-A165 Protein Binding Assay

Plates (96-well, Nunc Maxisorp; Thermo Fisher Scientific, Waltham, MA, USA) were coated with human Neuropilin-1-Fc (10 ng per well, Cat #50-101-8343, Fisher, Hampton, NH) and incubated at room temperature overnight.

The following day, the plates were washed and blocked with 3% BSA in PBS to minimize non-specific adsorptive binding to the plates. SARS-CoV2 Spike protein (100 nM, S1 domain aa16-685, Cat #Z03485, Genscript, Piscataway, NJ), EG00229 (Cat #6986, Tocris) or the indicated compounds were added at 12.5 μM and incubated for 30 min at room temperature prior to adding biotinylated human VEGF-A165 (Cat #BT293, R&D systems) at 10 nM. As a negative control, some wells received PBS containing 3% BSA. The plates were incubated at room temperature with shaking for 1 h. Next, the plates were washed with PBS to eliminate unbound protein. Bound biotinylated VEGF was detected by streptavidin-HRP (Cat #016-030-084, Jackson immunoResearch). Tetramethylbenzidine (Cat #DY999, R&D Systems, St. Louis, MO) was used as the colorimetric substrate. The optical density of each well was determined immediately, using a microplate reader (Multiskan Ascent; Thermo Fisher Scientific) set to 450 nm with a correction wavelength of 570 nm. Data were normalized to the background and to the signal detected for VEGF-A165 alone.

ii. In Cell Western for Detecting Inhibition of VEGFR2 Activation by VEGF-A165

Mouse neuron derived Cathecholamine A differentiated CAD (ECACC, Cat #08100805) were grown in standard cell culture conditions, 37° C. in 5% C₀₂. All media were supplemented with 10% fetal bovine serum (Hyclone) and 1% penicillin/streptomycin sulfate from 10,000 μg/ml stock. CAD cells were maintained in DMEM/F12 media. Cells were plated in a 96-well plate and left overnight. The next day, indicated compounds (at 12.5 μM) or SARS-CoV-2 Spike (100 nM, S1 domain) were added in CAD cell complete media supplemented with 1 nM of mouse VEGF-A165 (Cat #RP8672, Invitrogen) and left at 37° C. for 1 hour. Media were removed and the cells rinsed three times with PBS before fixation using ice cold methanol (5 min). Methanol was removed and cells were left to dry completely at room temperature. Anti-VEGFR2 pY1175 was used to detect the activation of the pathway triggered by VEGF-A165 in the cells. The antibody was added in PBS containing 3% BSA and left overnight at room temperature. The cells were washed three times with PBS and then incubated with Alexa Fluor® 790 AffiniPure Goat Anti-Rabbit IgG (Cat #111-655-144, Jackson immunoResearch) in PBS, 3% BSA for 1 hour at room temperature. Cells were washed three times with PBS and stained with DAPI. Plates were imaged on an Azure Sapphire apparatus. Wells that did not receive the primary antibody were used a negative control. The signal was normalized to the cell load in each well (using DAPI) and to control wells not treated with VEGF-A165.

iii. Cell and Viral Culture

Vero-E6-TMPRSS2 cells were cultured in high glucose DMEM supplemented with 10% FBS and 1% penicillin/streptomycin. Cells were supplemented with 20 μg/mL blasticidin (Invivogen, ant-b1-1) to maintain stable expression of TMPRSS2 during routine culture. Cells were maintained at 37° C. with 5% CO₂ and passaged every 2-3 days. These African green monkey Early kidney cells express NRP-1 which is 99.3% similar within its b1 domain to the human NRP-1. Homology models (not shown) also reveal no differences in NRP-1 passage between these species. Infectious VSV-eGFP-SARS-CoV-2 stock was a generous gift from Sean P. J. Whelan (Washington University, St. Louis, MO, USA). VSV-eGFP-SARS-CoV-2 was passaged once by infecting Vero-E6-TMPRSS2 cells at MOI=0.01 in DMEM+2% FBS and 1% penicillin/streptomycin for 72 hr at 34° C. Cell-free supernatant was collected and concentrated 10-fold through Amicon-Ultra 100 kDa MWCO spin filter units (Millipore UFC905008) prior to aliquoting and storage at −80° C. Titer was determined to be approximately 1×10⁷ PFU/mL as determined by median tissue culture infectious dose (TCIDso) assay on Vero-E6-TMPRSS2 cells.

iv. Screening Compounds for VSV-eGFP-SARS-CoV-2 Inhibition

African green monkey kidney (Vero)-E6-TMPRSS2 cells were plated at 15,000 cells per well in black/clear bottom 96-well tissue culture plates (Thermo Fisher Scientific 165305). The next day cells were infected ±0.001% DMSO, 25 μM compounds, or 68 nM recombinant Spike protein, at an MOI of 0.05 in 100 μL DMEM+10% FBS and 1% penicillin/streptomycin for 36 hrs at 37° C. prior to live cell fluorescent microscopy on a Nikon Eclipse Ti2 automated microscopy system with 4× objective and 488/532 nm filters. Sum GFP fluorescence intensity, normalized to cell count by HCS CellMask Blue (Thermo #H32720), was measured and for each well and plotted with Prism 6 (GraphPad Software). Significant differences were determined by RM one-way ANOVA followed by multiple comparisons test. An alpha of 0.05 was used to determine the statistical significance of the null-hypothesis.

v. Antagonism of NRP-1/VEGF-A Signaling

Tactile sensory thresholds were used to assess tactile allodynia via spared nerve injury (SNI)-induced mechanical allodynia in SNI rats (Sprague Dawley, male). Following a recovery period of seven days after implantation of intrathecal catheter, the spared nerve injury was induced. Under isoflurane anesthesia (5% induction, 2.0% maintenance in 2 L/min air), skin on the lateral surface of the left hind thigh was incised. The biceps femoris muscle was bluntly dissected to expose the three terminal branches of the sciatic nerve (Decosterd and Woolf, Pain 2000, 87(2), 149-158). Briefly, the common peroneal and tibial branches were tightly ligated with 5-0 silk, 2-3 mm of the nerves was removed below the ligations, with special care taken to avoid any damage to the sural nerve. Closure of the incision was made in two layers. The muscle was sutured once with 3-0 silk suture; skin was auto-clipped. Animals were allowed to recover for 12-14 days before the drug testing.

The assessment of tactile allodynia (i.e., a decreased threshold for paw withdrawal after probing with normally innocuous mechanical stimuli) involved testing the withdrawal threshold of the paw in response to probing with a series of calibrated fine (von Frey) filaments. Each filament was applied perpendicularly to the plantar surface of the paw of rats held in suspended wire mesh cages. The withdrawal threshold was determined by sequentially increasing and decreasing the stimulus strength (the ‘up and down’ method, and the data analyzed using the nonparametric method of Dixon (as described by Chaplan and Bach, J. Neurosci. Meth. 1994, 53, 55-63) with results expressed as the mean withdrawal threshold. The withdrawal threshold is a level that is statistically significant. Typically, greater than 50% deviation from the baseline could be allodynic.

Results

i. ELISA-Based NRP-1-VEGF-A165 Protein Binding Assay

The ability of hit compounds to interfere with the NRP-1NEGF-A interaction was evaluated using an enzyme linked immunosorbent assay (ELISA). Plates were coated with the extracellular domain of human NRP-1 (containing the a1a2 and the b1b2 regions) and added a selection of compounds described herein (based on SAR and commercial availability) to disrupt the NRP-1NEGF-A interaction. Three hit compounds blocked more than 50% of VEGF-A binding to NRP-1. This level of inhibition mimicked the level observed with SARS-CoV-2 Spike protein (Table 1). In comparison, EG00229's level of inhibition was outmatched by six of the nine compounds screened in this assay (Table 1). Thus, three of the synthetic compounds significantly inhibited the interaction between VEGF-A and NRP-1, confirming that they interfere with binding to the CendR site (Table 1).

Compounds for biochemical evaluation were selected such that each structural feature was complementary to the overall SAR. Relative inhibition in the ELISA is reported in Table 1. The results are based on compounds that were commercially available. Several compounds (12, 14, 15, 18, 19, 20) were not commercially available. Since many of those compounds can be synthesized in two to five steps, representatives structures will be synthesized during future SAR testing.

Inspection of docking scores and relative inhibition data in Table 1 reveals a correlation between predictions and ELISA data. The binding hypotheses were confirmed as lactams containing pyridone and pyrimidone cores. Some of the compounds disrupted the VEGF-A/NRP-1 interaction more effectively than EG00229 and to a similar extent as SARS-CoV-2 Spike protein.

Interference was greater for compounds with additional functional groups (1, 2, 4, 5 vs. 7, 8, 11). These results reveal that such a trend can be exploited for the design of new analogs that will expand on under-explored scaffolds (Series 3, compound 11).

ii. In Cell Western for Detecting Inhibition of VEGFR2 Activation by VEGF-A165

Next, the compounds were tested for their capacity to inhibit the activation of the VEGF-A pathway. VEGF-A binding to the dimeric complex of its receptor VEGFR2 and co-receptor NRP-1 triggers phosphorylation of the VEGFR2 cytoplasmic domain at Y1175. Using an in-cell Western assay, the compounds were tested for their ability to inhibit increased phosphorylation of VEGFR2 by VEGF-A. In this assay, VEGF-A doubled the level of VEGFR2 phosphorylation at Y1175 which could be blocked by SARS-CoV-2 Spike protein as well as by the reference compound EG00229 (FIG. 6). All nine tested compounds selected based on the virtual screening described herein, significantly blocked the VEGF-A stimulated increased phosphorylation of VEGFR2 (FIG. 6). In the absence of stimulation by VEGF-A, only one of the nine compounds (compound 4) showed inhibition of basal VEGFR2 phosphorylation. As mentioned above, several of the compound identified from the virtual screens exhibit features that are consistent with known kinase inhibitors which will be addressed during further development of these hits.

iii. Screening Compounds for VSV-eGFP-SARS-CoV-2 Inhibition

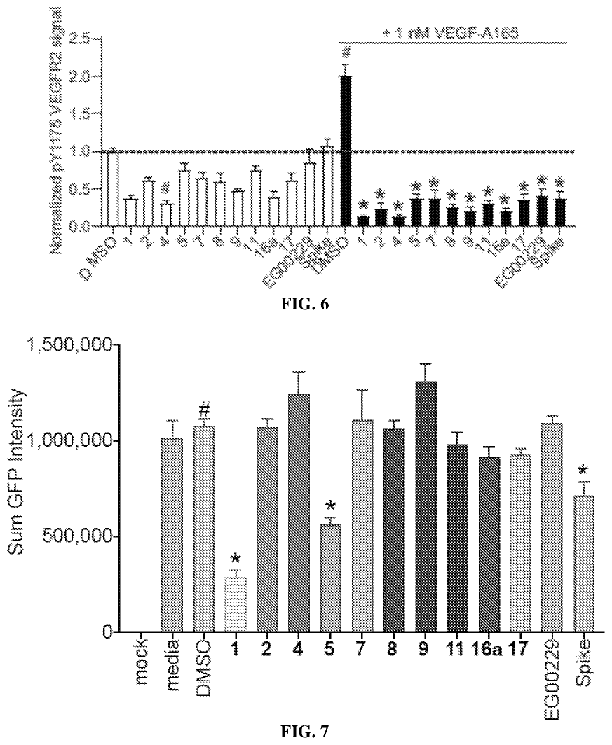

Finally, the compounds were screened for antiviral activity using a GFP-expressing vesicular stomatitis virus (VSV) recombinant protein, encoding the SARS-CoV-2 spike protein rather than the native envelope glycoprotein (Case, et al., Cell Host Microbe 2020, 28 (3), 475-485 e5). This VSV-eGFP-SARS-CoV-2 mimics SARS-CoV-2 and is a convenient BSL2 platform to assess SARS-CoV-2 Spike-dependency. Vero-E6-TMPRSS2 cells, which overexpress the transmembrane serine protease 2 (TMPRSS2)(Case, et al., Cell Host Microbe 2020, 28 (3), 475-485 e5), were infected in the presence of individual compounds or DMSO vehicle. GFP fluorescence was measured 36 hours post infection by automated microscopy. Two compounds (1 and 5) displayed >50% inhibition of VSV-eGFP-SARS-CoV-2 antiviral activity while another two compounds (16a, 17) demonstrated ˜15% inhibition (FIG. 7). Spike inhibited antiviral activity by ˜35% while the known NRP-1 inhibitor EG00229 was ineffective in this assay.

iv. Antagonism of NRP-1/VEGF-A Signaling

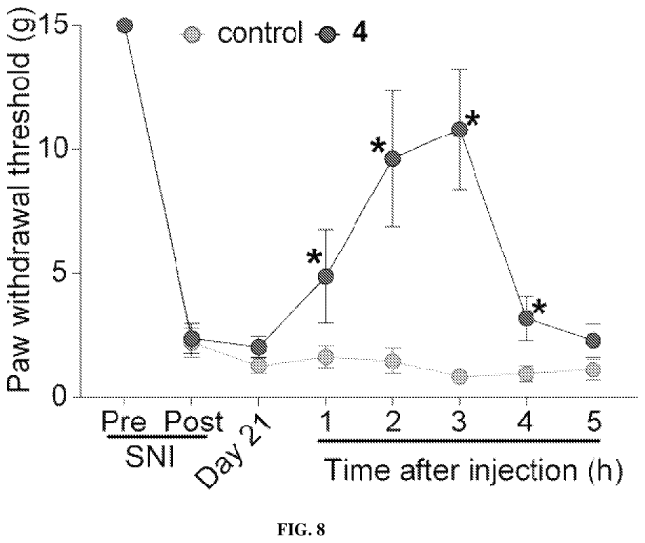

The results of the antagonism of NRP-1NEGF-A signaling using compound 4 are shown in FIG. 8. As shown, compound 4 reverses SNI-induced mechanical allodynia. Fourteen days following SNI, animals developed robust mechanical allodynia which was unchanged at day 21. At days 21, the animals were randomly assigned into the two groups and injected in an investigator-blinded manner with either vehicle control or compound 4. A time-course of tactile assessments revealed that control-injected animals were still allodynic over the four-hour time course. In contrast, SNI animals injected with compound 4 started to exhibit a reversal in their mechanical hypersensitivity (allodynia) by the first hour and this achieved maximal reversal at between 2 and 3 hours and then subsided thereafter.

### Example 3: Analyzing Features in Ligand-Receptor Interactions

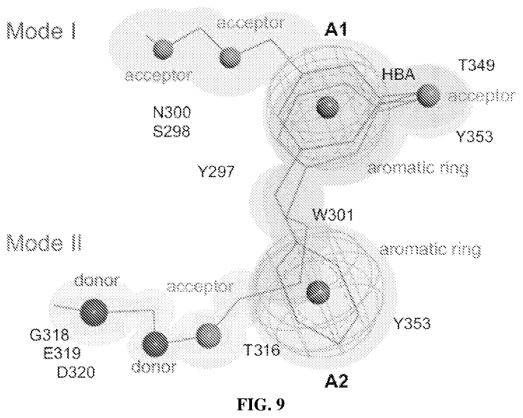

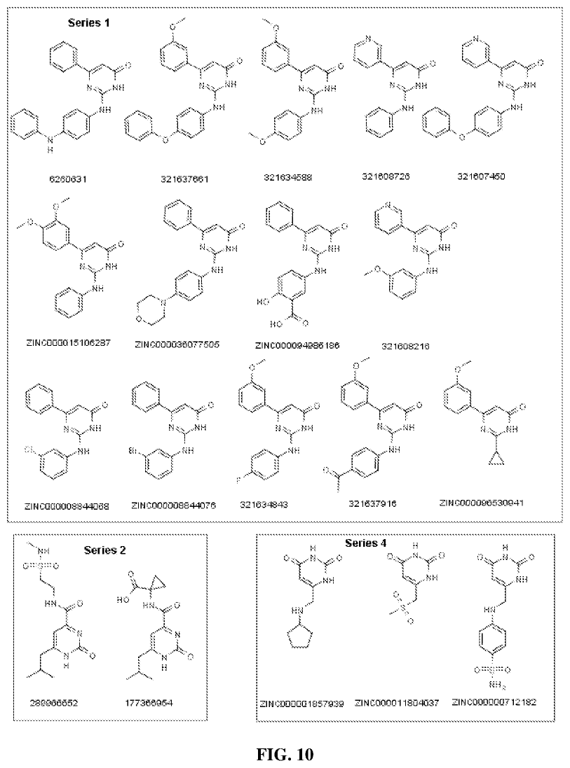

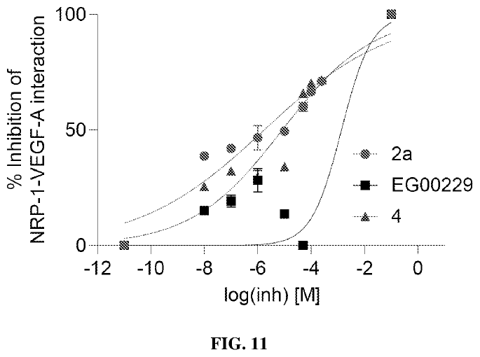

A pharmacophore model was derived from the identified ligands, considering both steric and electronic requirements (FIG. 9). Features identified in the pharmacophore models are the aromatic rings A1, A2, and the hydrogen bond acceptor HBA. This HBA is typically a carbonyl oxygen engaged in contacts with the hydroxyl groups of Tyr353 and Thr349. The aryl group in A1 directs the carbonyl oxygen of the HBA toward those residues. Alternatively, A1 can be presented in an edge-to-face contact with Tyr297. There are two additional acceptor sites on the opposite side of the A1 ring, relative to acceptor HBA. These can form hydrogen bonds with the side chain of the Asn300 amide or the Ser298 hydroxyl. The area between the HBA and these two additional acceptors (where the label A1 is located) is expected to accommodate a structural molecule of water (Appleton, et al., The EMBO journal 2007, 26 (23), 4902-12; Lee, et al., Structure 2003, 11 (1), 99-108; Mota, et al., The FEBS Journal 2018, 285 (7), 1290-1304; Powell, et al., J. Med. Chem. 2018, 61 (9), 4135-4154). This water has been proposed to be important in ligand binding as it may bridge interactions to Trp301 (Mota, et al., The FEBS Journal 2018, 285 (7), 1290-1304). However, the screens were conducted in the absence of water molecules. Nevertheless, it was observed that polar groups in several analogs occupy the position of this structural water and get involved in the corresponding hydrogen bond network, showing that they could displace it. Aromatic ring A2 is sandwiched in the hydrophobic box formed by residues Y353, W301, Y297, and the methyl group of T316. Binding mode II includes A1/HBA, A2, and features an expansion toward polar residues (E319, D320) and additional stabilizing contacts in the lower part of the pocket. It is this area (donor, donor, acceptor) which accepts the guanidino group of the CendR Arg and contains the open lower left pocket seen in Mode I (FIG. 4A).

### Example 4: Analyzing Features in Ligand-Receptor Interactions

### Materials and Methods

ELISA-Based NRP1-VEGF-A165 Protein Binding Assay

Plates (96-well, Nunc Maxisorp; Thermo Fisher Scientific, Waltham, MA, USA) were coated with human Neuropilin-1-Fc (200 ng per well, Cat #50-101-8343, Fisher, Hampton, NH) and incubated at 4° C. overnight. The following day, the plates were washed and blocked with 3% BSA in PBS to minimize non-specific adsorptive binding to the plates. EG00229 (Cat #6986, Tocris) or the indicated compounds were added at different concentrations and incubated for 30 min at room temperature prior to adding biotinylated human VEGF-A165 (Cat #BT10543-025, R&D systems) at 20 nM with 4 μg/ml of heparin. As a negative control, some wells received PBS containing 3% BSA and as a positive control only hVEGF-A165 was used. The plates were incubated at room temperature with shaking for 2 h. Next, the plates were washed with PBS 0.05% Tween to eliminate unbound protein. Bound biotinylated VEGF was detected by streptavidin-HRP (Cat #016-030-084, Jackson immunoResearch). Tetramethylbenzidine (Cat #DY999, R&D Systems, St. Louis, MO) was used as the colorimetric substrate. The optical density of each well was determined immediately, using a microplate reader (Multiskan Ascent; Thermo Fisher Scientific) set to 450 nm with a correction wavelength of 570 nm. Percentage of inhibition was calculated by the following formula:

$$\\ 100 \% - [ \frac{( S - N )}{( P - N )} * 100 \% ]$$

where S is the optical density measured in the wells with the compounds, N is the optical density measured in the negative control wells and P is the optical density in the positive control wells. The IC₅₀ was calculated using a non-linear regression function with GraphPad Prism Version 9. The data are presented as log (inhibitor) [M] versus normalized response-variable slope. Data are the means±S.E.M. of three independent experiments.

Results

The results above, it was demonstrated that interfering with the NRP-1NEGF-A signaling pathway using small molecules and natural products alleviates neuropathic pain (Moutal, et al., Pain 2021, 12(1), 243-252). Using these data, a second generation of NRP-1NEGF-A antagonists were developed and tested. Table 2 shows the second-generation compounds and their properties, such as molecular weight, purity, solubility, and IUPAC name. Table 3 shows the chemical structures of the second generation series of compounds.

TABLE 2
Properties of compound series

| Compound | | Molecular | Purity | | |
|---|---|---|---|---|---|
| name | ID | weight | (HPLC) | Solubility | IUPAC name |
| FFS-BRP- | 1a | 309.33 | 97.87% | DMSO | 2-[(2-amino-4- |
| TCG-1a | | | | | pyridyl)amino]-4-(3- |
| | | | | | methoxyphenyl)-1H- |
| | | | | | pyrimidin-6-one |
| FFS-BRP- | 1c | 309.33 | 95.27% | DMSO | 2-(3-hydroxyanilino)-4- |
| TCG-1c | | | | | (3-methoxyphenyl)-1H- |
| | | | | | pyrimidin-6-one |
| FFS-BRP- | 1v | 307.35 | 98.98% | DMSO | 2-anilino-4-(5-methoxy-2- |
| TCG-1v | | | | | methyl-phenyl)-1H- |
| | | | | | pyrimidin-6-one |
| FFS-BRP- | 2a | 239.28 | 99.48% | DMSO | N-(2-hydroxyethyl)-6- |
| TCG-2a | | | | | isobutyl-2-oxo-1H- |
| | | | | | pyrimidine-4- |
| | | | | | carboxamide |
| FFS-BRP- | 9c | 219.20 | 97.73% | DMSO | 6-(3-hydroxyanilino)-1H- |
| TCG-9C | | | | | pyrimidine-2,4-dione |
| FFS-BRP- | 1s | 294.31 | 99.81% | DMSO | 2-anilino-4-(2-methoxy-4- |
| TCG-1s | | | | | pyridyl)-1H-pyrimidin-6-one |
| FFS-BRP- | 1u | 294.31 | 97.48% | DMSO | 2-anilino-4-(6-methoxy-2- |
| TCG-1u | | | | | pyridyl)-1H-pyrimidin-6-one |
| FFS-BRP- | 1d | 344.76 | 99.06% | DMSO | 2-(4-chloro-3-hydroxy- |
| TCG-1d | | | | | anilino)-4-(2-methoxy-4- |
| | | | | | pyridyl)-1H-pyrimidin-6-one |
| FFS-BRP- | 1t | 283.29 | 99.31% | DMSO | 4-(3-methoxyphenyl)-2- |
| TCG-1t | | | | | (1H-pyrazol-3-ylamino)- |
| | | | | | 1H-pyrimidin-6-one |
| FFS-BRP- | 1b- | 293.33 | 99.80% | DMSO | 6-anilino-4-(3- |
| TCG-1b- | iso | | | | methoxyphenyl)-1H- |
| isomer | | | | | pyrimidin-2-one |
| FFS-BRP- | 1b | 293.33 | 98.34% | DMSO | 4-anilino-2-(3- |
| TCG-1b | | | | | methoxyphenyl)-1H- |
| | | | | | pyrimidin-6-one |
| FFS-BRP- | 1g′ | 326.31 | 99.09% | DMSO | 4-[[2-(3-methoxyphenyl)-6- |
| TCG-1g- | | | | | oxo-1H-pyrimidin-4- |
| Acid | | | | | yl]amino]-1H-pyrrole-2- |
| intermediate | | | | | carboxylic acid |
| FFS-BRP- | 1g′- | 326.31 | 97.41% | DMSO | 4-[[4-(3-methoxyphenyl)-6- |
| TCG-1g- | iso | | | | oxo-1H-pyrimidin-2- |
| isomer-Acid | | | | | yl]amino]-1H-pyrrole-2- |
| intermediate | | | | | carboxylic acid |

TABLE 3
Chemical structures of compound series

| ID | Structure |
|---|---|
| 1a | 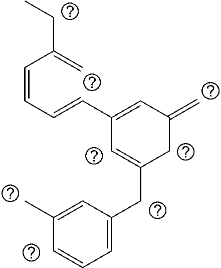 [SMILES: C=C1C=C(/C=C/C=C\C(=C)CC)C=C(Cc2cccc(C)c2)C1] |
| 1d | 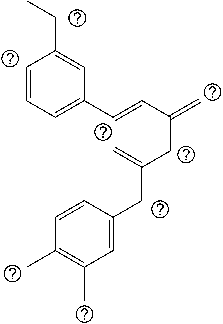 [SMILES: C=C(/C=C/c1cccc(CC)c1)CC(=C)Cc1ccc(C)c(C)c1] |
| 1c | 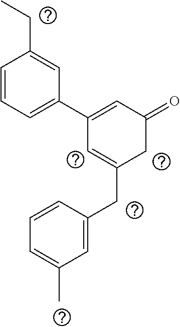 [SMILES: CCc1cccc(C2=CC(=O)CC(Cc3cccc(C)c3)=C2)c1] |
| 1t | 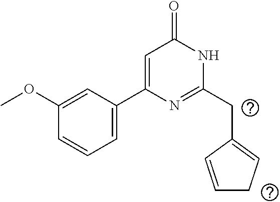 [SMILES: COc1cccc(-c2cc(=O)[nH]c(CC3=CCC=C3)n2)c1] |
| 1v | 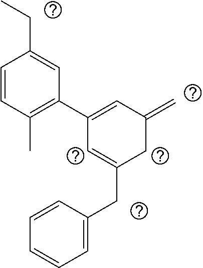 [SMILES: C=C1C=C(c2cc(CC)ccc2C)C=C(Cc2ccccc2)C1] |
| 1b- iso | 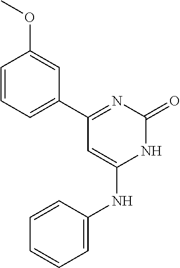 [SMILES: COc1cccc(-c2cc(Nc3ccccc3)[nH]c(=O)n2)c1] |
| 2a | 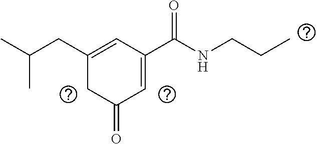 [SMILES: CCCNC(=O)C1=CC(=O)CC(CC(C)C)=C1] |
| 1b | 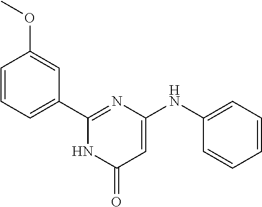 [SMILES: COc1cccc(-c2nc(Nc3ccccc3)cc(=O)[nH]2)c1] |
| 9c | 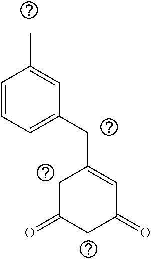 [SMILES: Cc1cccc(CC2=CC(=O)CC(=O)C2)c1] |
| 1g′ | 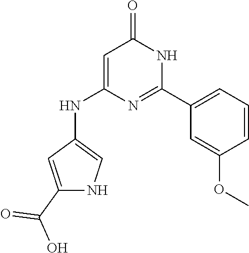 [SMILES: COc1cccc(-c2nc(Nc3c[nH]c(C(=O)O)c3)cc(=O)[nH]2)c1] |
| 1s | 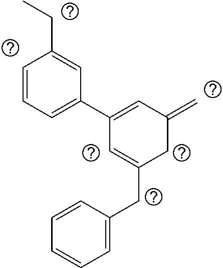 [SMILES: C=C1C=C(c2cccc(CC)c2)C=C(Cc2ccccc2)C1] |
| 1g′- iso | 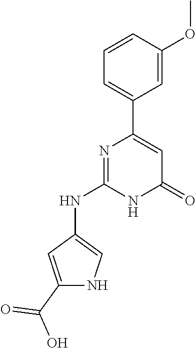 [SMILES: COc1cccc(-c2cc(=O)[nH]c(Nc3c[nH]c(C(=O)O)c3)n2)c1] |
| 1u | 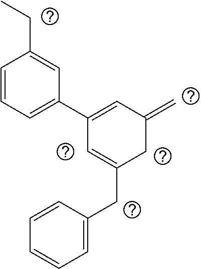 [SMILES: C=C1C=C(c2cccc(CC)c2)C=C(Cc2ccccc2)C1] |

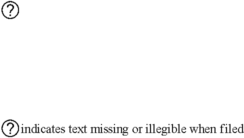

Additionally, an initial absorption, distribution, metabolism, and excretion (ADME) profile of the compound series was performed (Table 4). The results showed that compounds 2a and 9c had the best scores in all the assays; physicochemical screening (kinetic solubility and logD), metabolic stability (human and rodent microsomes half-life, T_{1/2}), cell proliferation and cytotoxicity in HepG2 cells (Cell Titer G u IC₅₀) (Table 4). Compounds 1g′ and 1g′-iso had good results for all assays except lipophilicity (distribution coefficient, logD) in which they had a lower but acceptable value.

TABLE 4
ADME profile of compound series

| | Kinetic | | micro- | micro- | micro- | HepG2 |
|---|---|---|---|---|---|---|
| | solu- | | somes | somes | somes | (48 h) |
| | bility | LogD | (human) | (rat) | (mouse) | CellTiterGlo |
| | (pH 7.4, | (oct/PBS, | T_{1/2} | T_{1/2} | T_{1/2} | IC₅₀ |
| ID | μM) | pH 7.4) | (min) | (min) | (min) | (μM) |
| 1a | 184 | 1.64 | >120 | 3.8 | 29.3 | >30 |
| 1c | 14.2 | 2.49 | >120 | 4.4 | 71.7 | >30 |
| 1v | 8.0 | 2.91 | >120 | 4.7 | >120 | >30 |
| 2a | >200 | 0.02 | >120 | >120 | >120 | >30 |
| 9c | >200 | 0.38 | >120 | >120 | >120 | >30 |
| 1s | 11.5 | 3.15 | 16.6 | 9.4 | 13.6 | >30 |
| 1u | 10.4 | 3.51 | 18.9 | 3.9 | 9.6 | >30 |
| 1d | 19.4 | 3.15 | 28.4 | 14.1 | 59.7 | >30 |
| 1t | 48.7 | 2.67 | 64.5 | 12.7 | 4.5 | >30 |
| 1b-iso | 4.7 | 2.78 | 102.5 | 4.4 | 22 | >30 |
| 1b | 3.5 | 3.78 | 14.8 | 8.3 | 9.4 | >30 |
| 1g′ | >200 | −0.62 | >120 | >120 | >120 | >30 |
| 1g′-iso | >200 | −0.38 | >120 | >120 | >120 | >30 |

Bold font: Optimal ADME parameter (meets or exceeds accepted criteria)
Regular font: Acceptable ADME parameters (less than optimal)
Italics font: Poor ADME parameters (far less than optimal)

Finally, the ability of the compounds to interfere with the NRP-1NEGF-A interaction was evaluated using an enzyme linked immunosorbent assay (ELISA), as previously described (Moutal, et al., Pain 2021, 12(1), 243-252). Plates were coated with the extracellular domain of human NRP-1 (containing the a1a2 and the b1b2 regions) and the compounds were added to disrupt the NRP-1NEGF-A interaction. The biotinylated human VEGF-A used in the experiments described above (Moutal, et al., Pain 2021, 12(1), 243-252) was discontinued. For the instant studies, biotinylated hVEGF-A (Cat #BT10543-025, R&D systems) was used. Because of that, the known NRP-1 antagonist EG00229 and compound 4 from the above series of inhibitors (Moutal, et al., Pain 2021, 12(1), 243-252) were also tested in the instant studies.

Representative concentration-response curves of NRP-1NEGF-A interaction are shown in FIG. 11. Calculated IC₅₀ values for the assay are shown in Table 5, as well as the goodness of fit (R²).

TABLE 5
Calculated IC₅₀ values of inhibiting the NRP-1/
VEGF-A interaction by the compound series

| Compound | μM | R² |
|---|---|---|
| 1a | 213.5 | 0.30 |
| 1c | 23.1 | 0.52 |
| 1v | 4.9 | 0.61 |
| 2a | 1.8 | 0.9 |
| 9c | 468.7 | 0.70 |
| 1s | 1034.0 | 0.61 |
| 1u | 369.3 | 0.59 |
| 1d | 134.4 | 0.68 |
| 1t | 127.7 | 0.63 |
| 1b-iso | 105.4 | 0.76 |
| 1b | 112.4 | 0.67 |
| 1g′ | 28.2 | 0.76 |
| 1g′-iso | 32.0 | 0.79 |
| 4 | 8.4 | 0.91 |
| EG00229 | 1371 | 0.77 |

The compounds were tested in a concentration range of 10 nM to 250 PM; n=3 replicates per concentration. Compounds 1c, 1v, 2a, 1g′ and 1g′-iso exhibited IC₅₀≤32 μM (Table 5). In comparison, compound 4 inhibited the NRP-1NEGF-A interaction with an IC₅₀ value of 8.4 μM, while compound 2a exhibited an IC₅₀ of 1.8 μM (Table 5). Thus, compound 2a significantly inhibited the interaction between VEGF-A and NRP-1, confirming that it competes for binding to NRP-1.

Those skilled in the art will recognize, or be able to ascertain using no more than routine experimentation, many equivalents to the specific embodiments of the invention described herein. Such equivalents are intended to be encompassed by the following claims.

1. A method of treating, preventing, or reducing neuropathic pain in a patient, comprising providing a formulation comprising a compound having the structure: 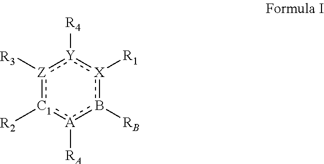
or a pharmaceutically acceptable salt thereof, in an effective amount to treat, prevent, or reduce the neuropathic pain,
wherein:
C₁ is a carbon atom,
A, B, X, Y, and Z are independently carbon or nitrogen,
A, B, C₁, X, Y, and Z are bonded to none, one, or two hydrogen atoms according to valency,
the dashed lines in Formula I denote the presence or absence of a bond,
R₁, R_{B}, R_{A}, R₂, R₃, and R₄ are independently absent, hydrogen, oxo- (═O), hydroxyl, substituted alkyl, unsubstituted alkyl, substituted alkenyl, unsubstituted alkenyl, substituted aryl, unsubstituted aryl, substituted heteroaryl, unsubstituted heteroaryl, substituted amino, unsubstituted amino, substituted C₃-C₂₀ cyclyl, unsubstituted C₃-C₂₀ cyclyl, —NR₅R₆, —C(O)NR₉R₁₀, —C(O)R₁₁, carboxyl, or sulfonyl, or ZR₃ and YR₄ together form a substituted heteroaryl,
R₅ and R₆ are independently hydrogen, unsubstituted alkyl, substituted alkyl, unsubstituted aryl, substituted aryl, substituted heteroaryl, or unsubstituted heteroaryl,
R₉ and R₁₀ are independently hydrogen, substituted alkyl, or unsubstituted alkyl, or NR₉R₁₀ together form a substituted heterocyclyl,
R₁₁ is hydroxyl, —SH, —NH₂, halogen, —CN, —OCN, —CNO, —NO₂, or —COOH, preferably, R₁₁ is hydroxyl, and
a) wherein the moiety 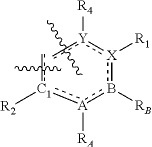
has the structure 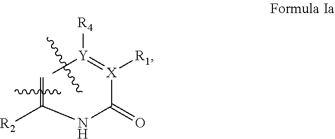
wherein
for Formula Ia, X is carbon, and Y is carbon or nitrogen,
1) R₁ is hydrogen, substituted alkyl, or unsubstituted alkyl,
R₂ is —NR₅R₆; unsubstituted heteroaryl; substituted heteroaryl; unsubstituted aryl; substituted aryl; unsubstituted C₃-C₂₀ cyclyl; substituted C₃-C₂₀ cyclyl; unsubstituted heterocyclyl; substituted heterocyclyl; substituted alkyl or unsubstituted alkyl,
wherein R₅ and R₆ are independently hydrogen; unsubstituted aryl; substituted aryl; unsubstituted heteroaryl; substituted heteroaryl unsubstituted C₃-C₂₀ cyclyl; substituted C₃-C₂₀ cyclyl; unsubstituted heterocyclyl; substituted heterocyclyl; unsubstituted alkyl, or substituted alkyl, preferably wherein at least one of R₅ and R₆ is unsubstituted aryl; substituted aryl; unsubstituted heteroaryl; substituted heteroaryl; unsubstituted C₃-C₂₀ cyclyl; substituted C₃-C₂₀ cyclyl; unsubstituted heterocyclyl; or substituted heterocyclyl,
R₄ is absent, hydrogen, substituted aryl, unsubstituted aryl (such as phenyl), substituted heteroaryl, unsubstituted heteroaryl, unsubstituted alkyl, substituted alkyl, or —NR₅R₆, wherein R₅ and R₆ are independently hydrogen; unsubstituted aryl; substituted aryl unsubstituted heteroaryl; substituted heteroaryl,
or
2) X—R₁ and Y—R₄ together form an unsubstituted heteroaryl
b) wherein the moiety 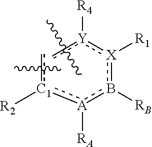
has the structure 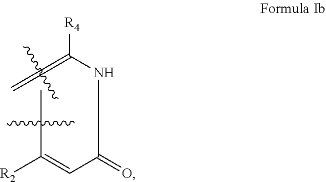
wherein
R₂ is —NR₅R₆ wherein R₅ and R₆ are independently hydrogen; unsubstituted aryl; substituted aryl, unsubstituted heteroaryl; substituted heteroaryl;
unsubstituted C₃-C₂₀ cyclyl; substituted C₃-C₂₀ cyclyl; unsubstituted heterocyclyl; substituted heterocyclyl; unsubstituted alkyl, or substituted alkyl; substituted aryl; unsubstituted heteroaryl; substituted heteroaryl; unsubstituted C₃-C₂₀ cyclyl; substituted C₃-C₂₀ cyclyl; unsubstituted heterocyclyl; or substituted heterocyclyl,
R₄ is hydrogen, substituted aryl unsubstituted aryl, or substituted heteroaryl,
c) wherein for Formula I, Z is carbon, and for Formula Ia, Y is carbon, X is carbon or nitrogen, and
R₂ is substituted alkyl, unsubstituted alkyl, —NR₅R₆, wherein R₅ and R₆ are independently hydrogen; substituted heteroaryl unsubstituted heteroaryl, substituted aryl, unsubstituted aryl, substituted C₃-C₂₀ cyclyl, unsubstituted C₃-C₂₀ cyclyl, substituted heterocyclyl, or unsubstituted heterocyclyl,
R₄ is —C(O)NR₉R₁₀, —C(O)R₁₁, substituted heteroaryl, unsubstituted heteroaryl, or substituted aryl,
wherein R₉ and R₁₀ are independently hydrogen, substituted alkyl, unsubstituted alkyl, or NR₉R₁₀ together form a substituted heterocyclyl, substituted heterocyclyl fused with heteroaryl, or substituted heterocyclyl fused with heteroaryl,
R₁₁ is hydroxyl, —SH, —NH₂, halogen, —CN, —OCN, —CNO, —NO₂, or —COOH,
d) wherein the moiety 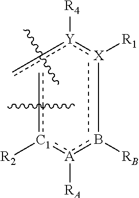
has the structure 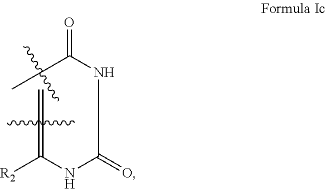
wherein
R₂ is substituted alkyl, carboxyl, sulfonyl, —NR₅R₆,
wherein R₅ and R₆ are independently hydrogen; substituted aryl unsubstituted aryl, unsubstituted heteroaryl, substituted heteroaryl, substituted C₃-C₂₀ cyclyl, unsubstituted C₃-C₂₀ cyclyl, substituted heterocyclyl, or unsubstituted heterocyclyl,
e) wherein Formula I has the structure 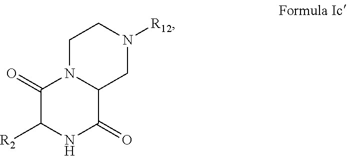
wherein
R₂ is substituted alkyl, or unsubstituted alkyl,
R₁₂ is —C(O)R₁₂ wherein R₁₂ is substituted alkyl,
f) wherein Formula I has the structure: 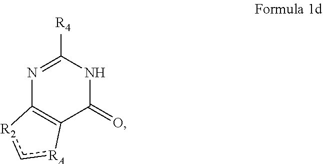
wherein
R₂ and R_{A} are nitrogen bonded to none or one hydrogen atom according to valency, and the dashed lines in Formula id denote the presence or absence of a bond,
R₄ is —NR₅R₆, wherein R₅ and R₆ are independently hydrogen, substituted alkyl, or unsubstituted alkyl, or
g) wherein Formula I has the structure: 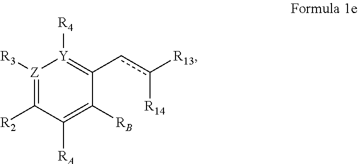
wherein for Formula 1e,
the dashed line denotes the presence or absence of a bond,
Y and Z are carbon,
R₂ and R₃ are hydroxyl, or ZR₃ and YR₄ together form a substituted heteroaryl ring,
R₄, R_{A}, and R_{B} are independently hydrogen, hydroxyl, unsubstituted alkyl, substituted alkyl, unsubstituted aryl, substituted aryl, unsubstituted heteroaryl, or substituted heteroaryl,
R₁₃ and R₁₄ are independently hydrogen, —C(O)R₁₅, or substituted alkenyl,
wherein R₁₅ is substituted alkyl, substituted alkoxy, substituted aroxy, or substituted heterocyclyl.

2. The method of claim 1, wherein Z is nitrogen, R₃ is absent, and for Formula Ia:
X is carbon and Y is carbon,
R₁ is hydrogen or substituted C₁-C₁₀alkyl,
R₂ is —NR₅R₆, unsubstituted heteroaryl, unsubstituted C₃-C₂₀ cyclyl, substituted heterocyclyl, substituted C₁-C₁₀ alkyl, substituted aryl, wherein R₅ and R₆ are independently hydrogen, unsubstituted aryl, substituted aryl, unsubstituted heteroaryl, substituted heteroaryl,
R₄ is hydrogen, substituted aryl, unsubstituted aryl, substituted heteroaryl, unsubstituted heteroaryl, unsubstituted C₁-C₁₀ alkyl, or —NR₅R₆, wherein R₅ and R₆ are independently hydrogen, or substituted heteroaryl, or
X—R₁ and Y—R₄ together form an unsubstituted heteroaryl.

3. The method of claim 2, wherein for Formula Ia
R₂ is —NR₅R₆, substituted aryl, or unsubstituted heteroaryl, and R₅ and R₆ are independently hydrogen, unsubstituted aryl, substituted aryl, or substituted heteroaryl, and
R₄ is hydrogen, substituted aryl, substituted heteroaryl, or —NR₅R₆, wherein R₅ and R₆ are independently hydrogen, or substituted heteroaryl.

4. (canceled)

5. The method of claim 1, wherein Z is nitrogen, R₃ is absent, and for Formula Ib,
R₂ is —NR₅R₆, wherein R₅ and R₆ are independently hydrogen; unsubstituted aryl; substituted heteroaryl, wherein at least one of R₅ and R₆ is unsubstituted aryl; substituted heteroaryl H, and
R₄ is substituted aryl.

6. The method of claim 1, wherein Z is nitrogen and R₃ is absent, and for Formula Ia,
Y is nitrogen and R₄ is absent,
R₁ is hydrogen, and
R₂ is —NR₅R₆, wherein R₅ and R₆ are independently hydrogen or substituted aryl.

7. The method of claim 1, wherein, for Formula I, Z is carbon,
R₂ is substituted C₁-C₁₀ alkyl, unsubstituted C₁-C₁₀ alkyl, —NR₅R₆, wherein R₅ and R₆ are independently hydrogen; unsubstituted aryl, substituted heteroaryl,
R₄ is —C(O)NR₉R₁₀, —C(O)R₁₁, substituted heteroaryl, unsubstituted heteroaryl, or substituted aryl,
wherein R₉ and R₁₀ are independently hydrogen, substituted alkyl, unsubstituted alkyl, or NR₉R₁₀ together form a substituted heterocyclyl, and
R₁₁ is hydroxyl, —SH, —NH₂, halogen, —CN, —OCN, —CNO, —NO₂, or —COOH.

8. (canceled)

9. The method of claim 7, wherein
R₂ is substituted C₁-C₁₀ alkyl, or —NR₅R₆, wherein R₅ and R₆ are independently hydrogen or unsubstituted aryl,
R₄ is —C(O)NR₉R₁₀, or substituted aryl, and
R₉ and R₁₀ are independently hydrogen or substituted alkyl.

10. The method of claim 1, wherein for Formula I, Z is carbon, R₃ is hydrogen or hydroxyl, and for Formula Ic,
R₂ is substituted C₁-C₁₀ alkyl, carboxyl, sulfonyl, —NR₅R₆,
wherein R₅ and R₆ are independently hydrogen; substituted aryl.

11. (canceled)

12. The method of claim 10, wherein
R₂ is substituted C₁-C₁₀ alkyl.

13.-15. (canceled)

16. The method of claim 1, wherein Formula I has the structure: 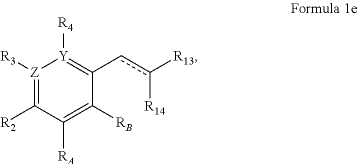
wherein for Formula 1e,
the dashed line denotes the presence or absence of a bond,
Y and Z are carbon,
R₂ and R₃ are hydroxyl, or ZR₃ and YR₄ together form a substituted heteroaryl ring substituted with a substituted aryl such as 3,4-dihydroxyphenyl,
R₄, R_{A}, and R_{B} are independently hydrogen, hydroxyl,
R₁₃ and R₁₄ are independently hydrogen, —C(O)R₁₅, substituted C₂-C₁₀ alkenyl,
wherein R₁₅ is substituted C₁-C₁₀ alkyl, substituted C₁-C₁₀ alkoxy substituted aroxy, substituted heterocyclyl.

17. The method of claim 16, wherein
the dashed line denotes the presence of a bond,
R₂ and R₃ are hydroxyl,
R₄, R_{A}, and R_{B} are hydrogen,
R₁₃ and R₁₄ are independently hydrogen or —C(O)R₁₅ wherein R₁₅ is substituted C₁-C₁₀ alkoxy.

18. The method of claim 1, wherein the effective amount is sufficient to inhibit phosphorylation of vascular endothelial growth factor receptor 2 (VEGFR2) compared to a control that does not contain the compound of Formula I, as determined by an in-cell Western assay that detects inhibition of VEGFR2 activation, optionally wherein the in-cell Western assay comprises activation of VEGFR2 with a peptide.

19.-21. (canceled)

22. The method of claim 1, wherein the effective amount is sufficient to reduce, ameliorate, or eliminate one or more symptoms associated with neuropathic pain in the patient.

23.-29. (canceled)

30. The method of claim 1, wherein the patient has neuropathic pain that stems from nerve trauma, peripheral nerve damage, central nerve damage, diabetes mellitus, small fiber neuropathy, multiple sclerosis, HIV infection, or a combination thereof.

31. A method of treating, preventing or reducing a viral infection in a patient, comprising providing a formulation comprising a compound having the structure: 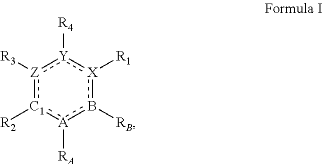
or a pharmaceutically acceptable salt thereof, in an effective amount to treat, prevent, or reduce the viral infection,
wherein:
C₁ is a carbon atom,
A, B, X, Y, and Z are independently carbon or nitrogen,
A, B, C₁, X, Y, and Z are bonded to none, one, or two hydrogen atoms according to valency,
the dashed lines in Formula I denote the presence or absence of a bond,
R₁, R_{B}, R_{A}, R₂, R₃, and R₄ are independently absent, hydrogen, oxo- (═O), hydroxyl, substituted alkyl, unsubstituted alkyl, substituted aryl, unsubstituted aryl, substituted heteroaryl, unsubstituted heteroaryl, substituted C₃-C₂₀ cyclyl, unsubstituted C₃-C₂₀ cyclyl, —NR₅R₆, —C(O)NR₉R₁₀,
R₅ and R₆ are independently hydrogen, unsubstituted aryl, substituted aryl, substituted heteroaryl, unsubstituted heteroaryl,
R₉ and R₁₀ are independently hydrogen, substituted alkyl, unsubstituted alkyl, or NR₉R₁₀ together form a substituted heterocyclyl, and
at least one of XR₁, BR_{B}, AR_{A}, C₁R₂, ZR₃, and YR₄ is —C(O)— and an adjacent XR₁, BR_{B}, AR_{A}, C₁R₂, ZR₃, and YR₄ is —NH—,
optionally wherein the effective amount is sufficient to reduce or block virus entry into a host cell, virus binding to neuropilin 1 (NRP-1), activates VEGF-A/NRP-1's signaling pathway, leading to reduction of viral titer over time, or a combination thereof.

32.-35. (canceled)

36. The method of claim 31, wherein the viral infection involves a host's respiratory system, optionally wherein the viral infection stems from severe acute respiratory syndrome coronavirus 2.

37.-39. (canceled)

40. A compound having the structure: 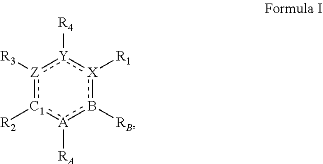
wherein
C₁ is a carbon atom,
A, B, X, Y, and Z are independently carbon or nitrogen,
A, B, C₁, X, Y, and Z are bonded to none, one, or two hydrogen atoms according to valency,
the dashed lines in Formula I denote the presence or absence of a bond, and
a) wherein the moiety 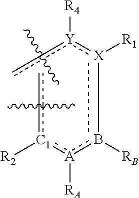
has the structure 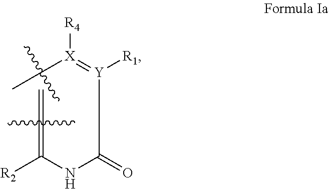
wherein
i) X is carbon, Y is carbon, Z is nitrogen, R₃ is absent, and for Formula Ia,
R₁ is hydrogen,
R₂ is —NR₅R₆, or substituted aryl, wherein R₅ and R₆ are independently hydrogen, unsubstituted aryl, substituted aryl, unsubstituted heteroaryl, substituted heteroaryl, and wherein one of R₅ and R₆ is hydrogen, and
R₄ is substituted aryl, substituted heteroaryl, or —NR₅R₆, wherein R₅ and R₆ are independently hydrogen, or substituted heteroaryl,
ii) X is nitrogen, R₁ is absent, Y is carbon, Z is carbon, and for Formula Ia,
R₂ is substituted C₁-C₁₀ alkyl, —NR₅R₆, wherein R₅ and R₆ are independently hydrogen; substituted heteroaryl; or unsubstituted aryl,
R₄ is —C(O)NR₉R₁₀, substituted heteroaryl, unsubstituted heteroaryl, or substituted aryl, and
wherein R₉ and R₁₀ are independently hydrogen, substituted alkyl, or NR₉R₁₀ together form a substituted heterocyclyl,
b) wherein the moiety 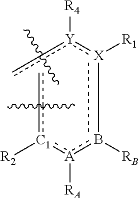
has the structure 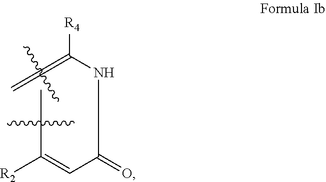
wherein
Z is nitrogen, R₃ is absent, and for Formula Ib,
R₂ is —NR₅R₆ wherein R₅ and R₆ are independently hydrogen; unsubstituted aryl; substituted heteroaryl,
R₄ is substituted aryl, or
c) wherein the moiety 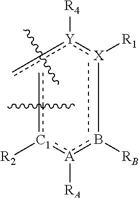
has the structure 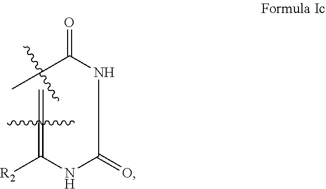
wherein
for Formula I, Z is carbon, R₃ is hydrogen,
R₂ is substituted C₁-C₁₀ alkyl; —NR₅R₆, wherein R₅ and R₆ are independently hydrogen; substituted aryl.

41. The compound of claim 40, having a structure:

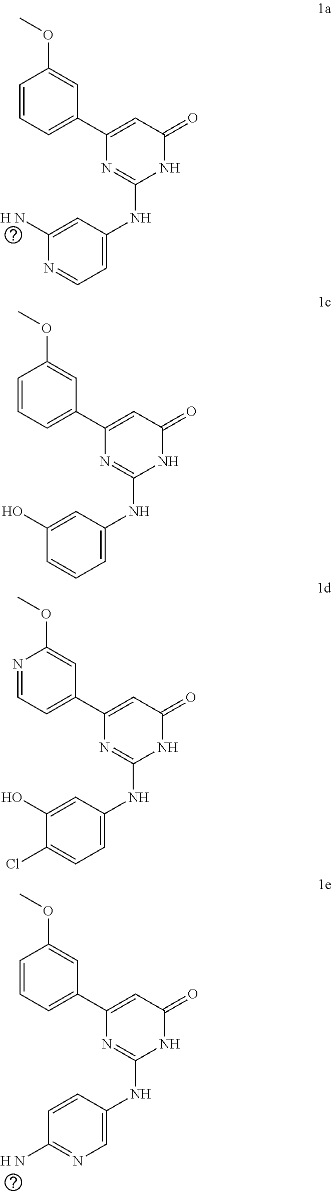

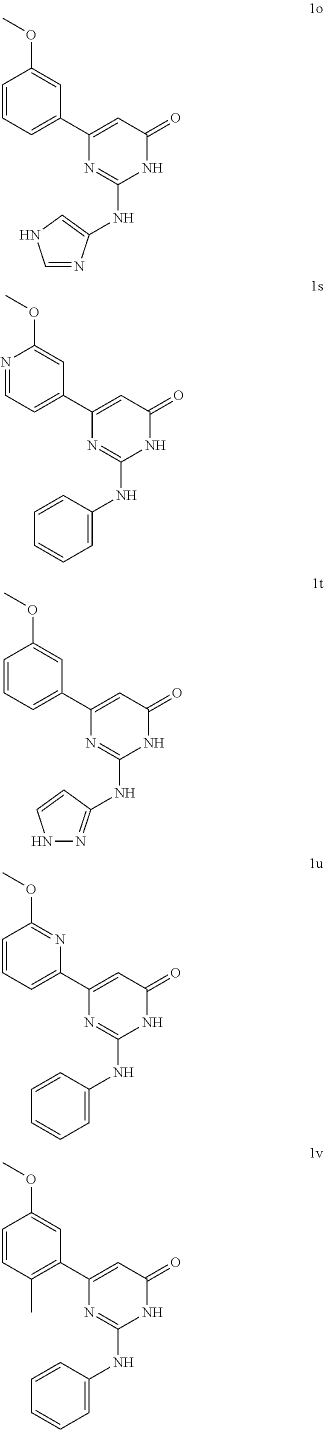 [SMILES: COc1cccc(-c2cc(=O)[nH]c(Nc3c[nH]cn3)n2)c1 ; COc1cc(-c2cc(=O)[nH]c(Nc3ccccc3)n2)ccn1 ; COc1cccc(-c2cc(=O)[nH]c(Nc3cc[nH]n3)n2)c1 ; COc1cccc(-c2cc(=O)[nH]c(Nc3ccccc3)n2)n1 ; COc1ccc(C)c(-c2cc(=O)[nH]c(Nc3ccccc3)n2)c1]

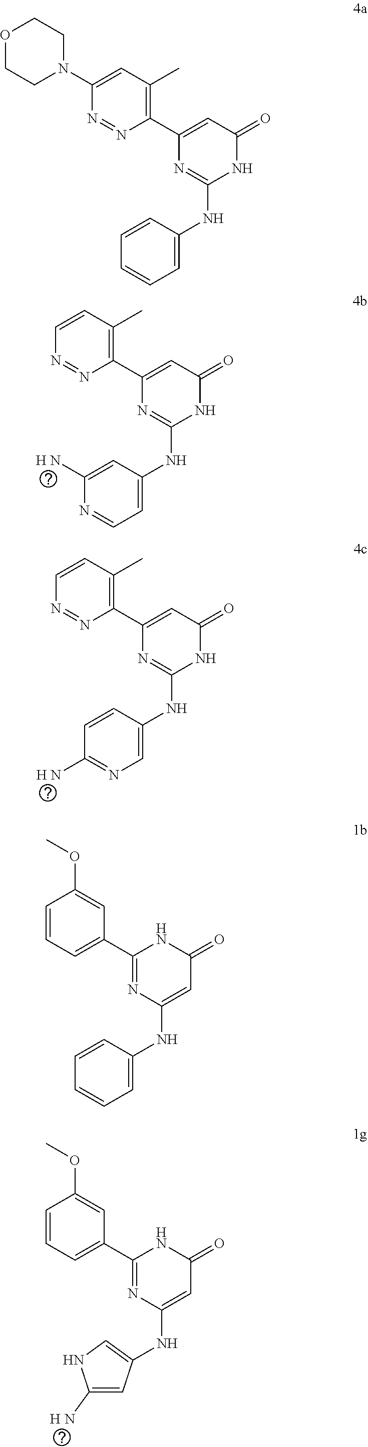

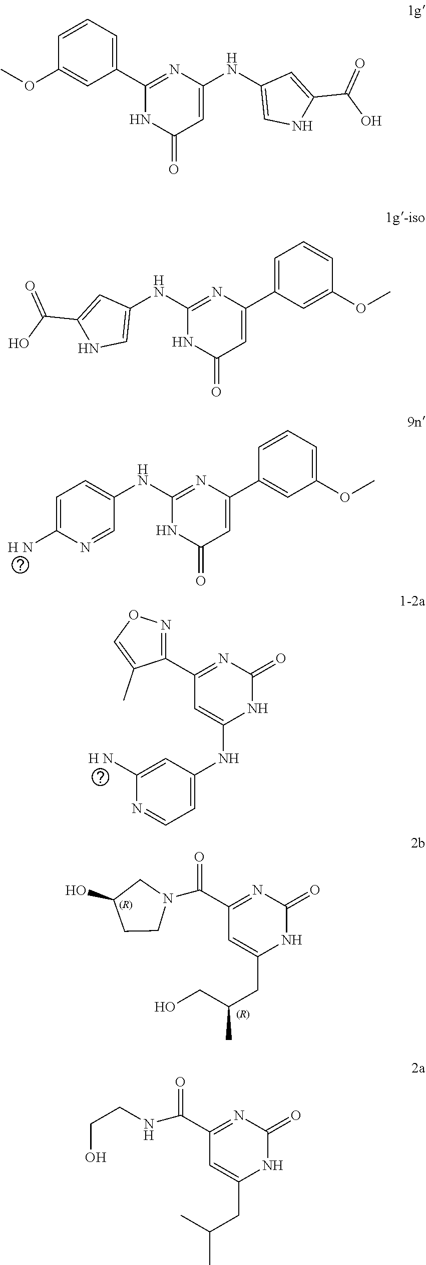

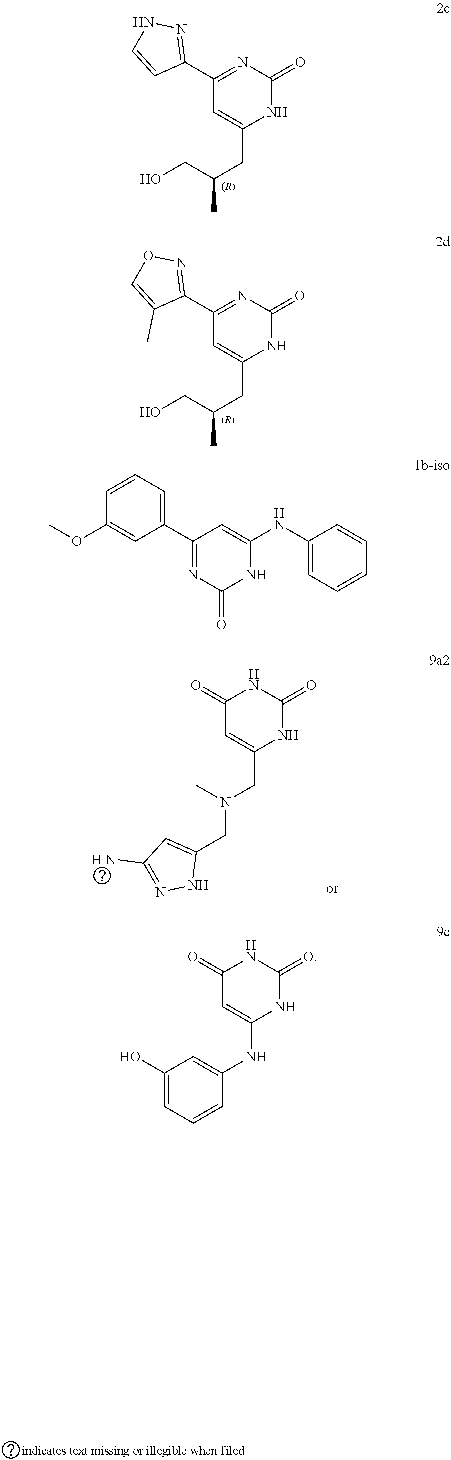

42. A formulation comprising the compound of claim 40, or a pharmaceutically acceptable salt thereof, in a pharmaceutically acceptable carrier.

43. (canceled)

44. A method of modulating NRP-1-mediated signaling in a subject in need thereof comprising administering the subject an effective amount of the compound of claim 40 to reduce NRP-1 signaling in cells of the subject.

45.-56. (canceled)
